SEQUENCE LISTING (87 sequences)

SEQ ID NO: 1 - protein, 8 aa, Artificial Sequence
  Description of Artificial Sequence: Synthetic peptide
GYTFTSYW

SEQ ID NO: 2 - protein, 8 aa, Artificial Sequence
  Description of Artificial Sequence: Synthetic peptide
IDPSDSYT

SEQ ID NO: 3 - protein, 13 aa, Artificial Sequence
  Description of Artificial Sequence: Synthetic peptide
NSWFAYWGQGTLV

SEQ ID NO: 4 - protein, 10 aa, Artificial Sequence
  Description of Artificial Sequence: Synthetic peptide
QSVDYDGDSY

SEQ ID NO: 5 - protein, 3 aa, Artificial Sequence
  Description of Artificial Sequence: Synthetic peptide
AAS

SEQ ID NO: 6 - protein, 9 aa, Artificial Sequence
  Description of Artificial Sequence: Synthetic peptide
QQSNEDPFT

SEQ ID NO: 7 - protein, 119 aa, Artificial Sequence
  Description of Artificial Sequence: Synthetic polypeptide
EVQLQESGAELVKPGASVKMSCKASGYTFTSYWMHWVKQRPGPGLEWIGAIDPSDSYTGYNQKFKGKATLTVDTSSSTAYMHLSSLTSEDSAVYFCTRSYYGNSWFAYWGQGTLVTVSA

SEQ ID NO: 8 - DNA, 357 bp, Artificial Sequence
  Description of Artificial Sequence: Synthetic polynucleotide
gaggtgcagctgcaggagtctggggctgagctggtgaagcctggggcttcagtgaagatgtcctgcaaggcttctggctacaccttcaccagctactggatgcactgggtgaagcagaggcctggaccaggccttgagtggatcggagcaattgatccttcagatagttatactggctacaatcaaaagttcaagggcaaggccacattgactgtagacacatcctccagcacagcctacatgcacctcagcagcctgacatctgaggactctgcggtctatttctgtacaagaagctactatggtaactcctggtttgcttactggggccaagggactctggtcactgtctctgca

SEQ ID NO: 9 - protein, 110 aa, Artificial Sequence
  Description of Artificial Sequence: Synthetic polypeptide
IVLTQSPASLAVSLGQRATISCKASQSVDYDGDSYINWYQQKPGQPPKLLIYAASNLESGIPARFSASGSGTDFTLNIHPVEEEDVAAYYCQQSNEDPFTFGSGTRLEIK

SEQ ID NO: 10 - DNA, 333 bp, Artificial Sequence
  Description of Artificial Sequence: Synthetic polynucleotide
acattgtgctgacccaatctccagcttctttggctgtgtctctagggcagagggccaccatctcctgcaaggccagccaaagtgttgattatgatggtgatagttatataaactggtaccaacagaaaccaggacagccacccaaactcctcatctatgctgcatccaatctagaatctggaatcccagccaggtttagtgccagtgggtctgggacagacttcaccctcaacatccatcctgtggaggaggaggatgttgcagcctattactgtcagcaaagtaatgaggacccattcacgttcggctcggggacaaggttggaaataaaac

SEQ ID NO: 11 - protein, 8 aa, Artificial Sequence
  Description of Artificial Sequence: Synthetic peptide
GYTFTSYW

SEQ ID NO: 12 - protein, 8 aa, Artificial Sequence
  Description of Artificial Sequence: Synthetic peptide
IDPSDSYT

SEQ ID NO: 13 - protein, 13 aa, Artificial Sequence
  Description of Artificial Sequence: Synthetic peptide
NSWFDYWGQGTLV

SEQ ID NO: 14 - protein, 10 aa, Artificial Sequence
  Description of Artificial Sequence: Synthetic peptide
QSVDYEGDSY

SEQ ID NO: 15 - protein, 3 aa, Artificial Sequence
  Description of Artificial Sequence: Synthetic peptide
AAS

SEQ ID NO: 16 - protein, 9 aa, Artificial Sequence
  Description of Artificial Sequence: Synthetic peptide
QQSNEDPFT

SEQ ID NO: 17 - protein, 119 aa, Artificial Sequence
  Description of Artificial Sequence: Synthetic polypeptide
EVQLQESGAELVKPGASVKMSCKASGYTFTSYWMHWVKQRPGQGLEWIGAIDPSDSYTGYNQKFKGKATLTVDTSSSTAYMHLSSLTSEDSAVYFCTRSYYGNSWFDYWGQGTLVTVSA

SEQ ID NO: 18 - DNA, 357 bp, Artificial Sequence
  Description of Artificial Sequence: Synthetic polynucleotide
gaggtgcagctgcaggagtctggggctgagctggtgaagcctggggcttcagtgaagatgtcctgcaaggcttctggctacaccttcaccagctactggatgcactgggtgaagcagaggcctggacaaggccttgagtggatcggagcaattgatccttcagatagttatactggctacaatcaaaagttcaagggcaaggccacattgactgtagacacatcctccagcacagcctacatgcacctcagcagcctgacatctgaggactctgcggtctatttctgtacaagaagctactatggtaactcctggtttgattactggggccaagggactctggtcactgtctctgca

SEQ ID NO: 19 - protein, 111 aa, Artificial Sequence
  Description of Artificial Sequence: Synthetic polypeptide
DIVLTQSPASLAVSLGQRATISCKASQSVDYEGDSYMNWYQQKPGQPPKLLIYAASNLESGIPARFSGSGSGTDFTLNIHPVEEEDAATYHCQQSNEDPFTFGGGTKLEIK

SEQ ID NO: 20 - DNA, 333 bp, Artificial Sequence
  Description of Artificial Sequence: Synthetic polynucleotide
gacattgtgctgacccaatctccagcttctttggctgtgtctctagggcagagggccaccatctcctgcaaggccagccaaagtgttgattatgaaggtgatagttatatgaactggtaccaacagaaaccaggacagccacccaaactcctcatctatgctgcatccaatctagaatctggaatcccagccaggtttagtggcagtgggtctgggacagacttcaccctcaacatccatcctgtggaggaggaggatgctgcaacctatcactgtcagcaaagtaatgaggacccgttcacgttcggaggggggaccaagttggaaataaaa

SEQ ID NO: 21 - protein, 8 aa, Artificial Sequence
  Description of Artificial Sequence: Synthetic peptide
GYTFTSYW

SEQ ID NO: 22 - protein, 7 aa, Artificial Sequence
  Description of Artificial Sequence: Synthetic peptide
DPSDSYT

SEQ ID NO: 23 - protein, 12 aa, Artificial Sequence
  Description of Artificial Sequence: Synthetic peptide
SWFAYWGQGTLV

SEQ ID NO: 24 - protein, 10 aa, Artificial Sequence
  Description of Artificial Sequence: Synthetic peptide
QSVDYDGDSY

SEQ ID NO: 25 - protein, 3 aa, Artificial Sequence
  Description of Artificial Sequence: Synthetic peptide
VAS

SEQ ID NO: 26 - protein, 9 aa, Artificial Sequence
  Description of Artificial Sequence: Synthetic peptide
QQSNEDPFT

SEQ ID NO: 27 - protein, 119 aa, Artificial Sequence
  Description of Artificial Sequence: Synthetic polypeptide
EVQLQESGAELVKPGASVKMSCKASGYTFTSYWMHWVKQRPGQGLEWIGAIDPSDSYTGYNQKFKGKATLTVDTSSSTAYMHLSSLTSEDSAVYFCTRSYYGNSWFAYWGQGTLVTVSA

SEQ ID NO: 28 - DNA, 357 bp, Artificial Sequence
  Description of Artificial Sequence: Synthetic polynucleotide
gaggtgcagctgcaggagtctggggctgagctggtgaagcctggggcttcagtgaagatgtcctgcaaggcttctggctacaccttcaccagctactggatgcactgggtgaagcagaggcctggacaaggccttgagtggatcggagcaattgatccttcagatagttatactggctacaatcaaaagttcaagggcaaggccacattgactgtagacacatcctccagcacagcctacatgcacctcagcagcctgacatctgaggactctgcggtctatttctgtacaagaagctactatggtaactcctggtttgcttactggggccaagggactctggtcactgtctctgca

SEQ ID NO: 29 - protein, 111 aa, Artificial Sequence
  Description of Artificial Sequence: Synthetic polypeptide
DIVLTQSPASLAVSLGQRATISCKASQSVDYDGDSYMNWYQQKPGQPPKLLIYVASNLESGIPARFSGSGSGTDFTLNIHPVEEEDAATYYCQQSNEDPFTFGSGTKLEIN

SEQ ID NO: 30 - DNA, 333 bp, Artificial Sequence
  Description of Artificial Sequence: Synthetic polynucleotide
gacattgtgctgacccaatctccagcttctttggctgtgtctctagggcagagggccaccatctcctgcaaggccagccaaagtgttgattatgatggtgatagttatatgaactggtaccaacagaaaccaggacagccacccaaactcctcatttatgttgcatccaatctagaatctggaatcccagccaggtttagtggcagtgggtctgggacagacttcaccctcaacatccatcctgtggaggaggaggatgctgcaacctattactgtcagcaaagtaatgaggacccattcacgttcggctcggggacaaagttggaaataaac

SEQ ID NO: 31-39 - skipped (9 entries)

SEQ ID NO: 40 - DNA, 1011 bp, Homo sapiens
cgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctagatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaagg

SEQ ID NO: 41 - protein, 337 aa, Homo sapiens
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRR

SEQ ID NO: 42 - DNA, 63 bp, Artificial Sequence
  Description of Artificial Sequence: Synthetic oligonucleotide
atggccctgcctgtgacagccctgctgctgcctctggctctgctgctgcatgccgctagaccc

SEQ ID NO: 43 - protein, 21 aa, Artificial Sequence
  Description of Artificial Sequence: Synthetic peptide
MALPVTALLLPLALLLHAARP

SEQ ID NO: 44 - DNA, 15 bp, Artificial Sequence
  Description of Artificial Sequence: Synthetic oligonucleotide
ggtggcggaggttct

SEQ ID NO: 45 - protein, 5 aa, Artificial Sequence
  Description of Artificial Sequence: Synthetic peptide
GGGGS

SEQ ID NO: 46 - DNA, 30 bp, Artificial Sequence
  Description of Artificial Sequence: Synthetic oligonucleotide
ggtggcggaggttctggaggtggaggttcc

SEQ ID NO: 47 - protein, 10 aa, Artificial Sequence
  Description of Artificial Sequence: Synthetic peptide
GGGGSGGGGS

SEQ ID NO: 48 - DNA, 45 bp, Artificial Sequence
  Description of Artificial Sequence: Synthetic oligonucleotide
ggaggaggtggtagtggtggaggaggaagtggaggaggaggaagt

SEQ ID NO: 49 - protein, 15 aa, Artificial Sequence
  Description of Artificial Sequence: Synthetic peptide
GGGGSGGGGSGGGGS

SEQ ID NO: 50 - DNA, 60 bp, Artificial Sequence
  Description of Artificial Sequence: Synthetic oligonucleotide
ggaggaggtggtagtggtggaggaggaagtggtggcggaggttctggaggtggaggttcc

SEQ ID NO: 51 - protein, 20 aa, Artificial Sequence
  Description of Artificial Sequence: Synthetic peptide
GGGGSGGGGSGGGGSGGGGS

SEQ ID NO: 52 - DNA, 135 bp, Artificial Sequence
  Description of Artificial Sequence: Synthetic polynucleotide
acaactactccagcaccacgaccaccaacacctgctccaactatcgcatctcaaccactttctctacgtccagaagcatgccgaccagctgcaggaggtgcagttcatacgagaggtctagatttcgcatgtgat

SEQ ID NO: 53 - protein, 45 aa, Artificial Sequence
  Description of Artificial Sequence: Synthetic polypeptide
TTTPAPRPPTPAPTIASQPLSLRPEACRPAAGGAVHTRGLDFACD

SEQ ID NO: 54 - DNA, 141 bp, Artificial Sequence
  Description of Artificial Sequence: Synthetic polynucleotide
aagcccacaactactccagcaccacgaccaccaacacctgctccaactatcgcatctcaaccactttctctacgtccagaagcatgccgaccagctgcaggaggtgcagttcatacgagaggtctagatttcgcatgtgat

SEQ ID NO: 55 - protein, 47 aa, Artificial Sequence
  Description of Artificial Sequence: Synthetic polypeptide
KPTTTPAPRPPTPAPTIASQPLSLRPEACRPAAGGAVHTRGLDFACD

SEQ ID NO: 56 - DNA, 174 bp, Artificial Sequence
  Description of Artificial Sequence: Synthetic polynucleotide
ttcagccacttcgtgccggtcttcctgccagcgaagcccacaactactccagcaccacgaccaccaacacctgctccaactatcgcatctcaaccactttctctacgtccagaagcatgccgaccagctgcaggaggtgcagttcatacgagaggtctagatttcgcatgtgat

SEQ ID NO: 57 - protein, 58 aa, Artificial Sequence
  Description of Artificial Sequence: Synthetic polypeptide
FSHFVPVFLPAKPTTTPAPRPPTPAPTIASQPLSLRPEACRPAAGGAVHTRGLDFACD

SEQ ID NO: 58 - DNA, 72 bp, Artificial Sequence
  Description of Artificial Sequence: Synthetic oligonucleotide
attgaagttatgtatcctcctccttacctagacaatgagaagagcaatggaaccattatccatgtgaaaggg

SEQ ID NO: 59 - protein, 24 aa, Artificial Sequence
  Description of Artificial Sequence: Synthetic peptide
IEVMYPPPYLDNEKSNGTIIHVKG

SEQ ID NO: 60 - DNA, 36 bp, Artificial Sequence
  Description of Artificial Sequence: Synthetic oligonucleotide
gagtccaaatatggtcccccatgcccatcatgccca

SEQ ID NO: 61 - protein, 12 aa, Artificial Sequence
  Description of Artificial Sequence: Synthetic peptide
ESKYGPPCPSCP

SEQ ID NO: 62 - DNA, 369 bp, Artificial Sequence
  Description of Artificial Sequence: Synthetic polynucleotide
gagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttccaaagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaaggg

SEQ ID NO: 63 - protein, 123 aa, Artificial Sequence
  Description of Artificial Sequence: Synthetic polypeptide
ESKYGPPCPSCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKG

SEQ ID NO: 64 - DNA, 687 bp, Artificial Sequence
  Description of Artificial Sequence: Synthetic polynucleotide
gagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttccaaagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctccgggtaaa

SEQ ID NO: 65 - protein, 229 aa, Artificial Sequence
  Description of Artificial Sequence: Synthetic polypeptide
ESKYGPPCPSCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 66 - DNA, 981 bp, Artificial Sequence
  Description of Artificial Sequence: Synthetic polynucleotide
gctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttccaaagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctccgggtaaa

SEQ ID NO: 67 - protein, 327 aa, Artificial Sequence
  Description of Artificial Sequence: Synthetic polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 68 - DNA, 72 bp, Artificial Sequence
  Description of Artificial Sequence: Synthetic oligonucleotide
atctacatctgggcaccattggctgggacttgtggtgtccttctcctatcactggttatcaccctttactgc

SEQ ID NO: 69 - protein, 24 aa, Artificial Sequence
  Description of Artificial Sequence: Synthetic peptide
IYIWAPLAGTCGVLLLSLVITLYC

SEQ ID NO: 70 - DNA, 84 bp, Artificial Sequence
  Description of Artificial Sequence: Synthetic oligonucleotide
atctacatctgggcaccattggctgggacttgtggtgtccttctcctatcactggttatcaccctttactgcaaccacaggaac

SEQ ID NO: 71 - protein, 28 aa, Artificial Sequence
  Description of Artificial Sequence: Synthetic peptide
IYIWAPLAGTCGVLLLSLVITLYCNHRN

SEQ ID NO: 72 - DNA, 120 bp, Artificial Sequence
  Description of Artificial Sequence: Synthetic polynucleotide
aaacacctttgtccaagtcccctatttcccggaccttctaagcccttttgggtgctggtggtggttggtggagtcctggcttgctatagcttgctagtaacagtggcctttattattttc

SEQ ID NO: 73 - protein, 40 aa, Artificial Sequence
  Description of Artificial Sequence: Synthetic polypeptide
KHLCPSPLFPGPSKPFWVLVVVGGVLACYSLLVTVAFIIF

SEQ ID NO: 74 - DNA, 123 bp, Artificial Sequence
  Description of Artificial Sequence: Synthetic polynucleotide
agaagtaaaagaagtaggctacttcatagtgattacatgaatatgactcctcgacgacctggtcccacccgtaagcattatcagccctatgcaccaccacgagatttcgcagcctatcgctcc

SEQ ID NO: 75 - protein, 41 aa, Artificial Sequence
  Description of Artificial Sequence: Synthetic polypeptide
RSKRSRLLHSDYMNMTPRRPGPTRKHYQPYAPPRDFAAYRS

SEQ ID NO: 76 - DNA, 126 bp, Artificial Sequence
  Description of Artificial Sequence: Synthetic polynucleotide
aaacgaggtagaaaaaaacttctttatatattcaaacaaccatttatgagaccagtacaaactactcaagaggaagatggatgtagttgtcgatttccagaagaagaagaaggaggatgtgaactg

SEQ ID NO: 77 - protein, 112 aa, Artificial Sequence
  Description of Artificial Sequence: Synthetic polypeptide
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 78 - DNA, 111 bp, Artificial Sequence
  Description of Artificial Sequence: Synthetic polynucleotide
aggcgcgaccagcggctgccacctgatgcacacaagccaccaggaggaggctctttccggaccccaatccaggaggagcaggcagacgcacacagcacactggccaagatc

SEQ ID NO: 79 - protein, 37 aa, Artificial Sequence
  Description of Artificial Sequence: Synthetic polypeptide
RRDQRLPPDAHKPPGGGSFRTPIQEEQADAHSTLAKI

SEQ ID NO: 80 - DNA, 336 bp, Artificial Sequence
  Description of Artificial Sequence: Synthetic polynucleotide
agagttaaatttagcagaagtgcagatgctcctgcgtataaacagggtcaaaaccaactatataatgaactaaatctaggacgaagagaagaatatgatgttttagataaaagacgtggtcgagatcctgaaatgggaggaaaacctagaagaaaaaatcctcaagaaggcctatataatgaactacaaaaagataagatggcagaagcttatagtgaaattggaatgaaaggagaacgtcgtagaggtaaaggtcatgatggtctttatcaaggtcttagtacagcaacaaaagatacatatgatgcacttcatatgcaagcacttccacctcgt

SEQ ID NO: 81 - protein, 111 aa, Artificial Sequence
  Description of Artificial Sequence: Synthetic polypeptide
RVKFSRSADAPAYKQGQNQLYNELNLGRREEYDVLDKRRGRDPEMGGKPRRKNPQEGLYELQKDKMAEAYSEIGMKGERRRGKGHDGLYQGLSTATKDTYDALHMQALPPR

SEQ ID NO: 82 - DNA, 717 bp, Artificial Sequence
  Description of Artificial Sequence: Synthetic polynucleotide
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 83 - protein, 239 aa, Artificial Sequence
  Description of Artificial Sequence: Synthetic polypeptide
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 84 - DNA, 54 bp, Artificial Sequence
  Description of Artificial Sequence: Synthetic oligonucleotide
gagggcagaggcagtctgctgacatgcggtgacgtggaagagaatcccggccct

SEQ ID NO: 85 - protein, 18 aa, Artificial Sequence
  Description of Artificial Sequence: Synthetic peptide
EGRGSLLTCGDVEENPGP

SEQ ID NO: 86 - DNA, 63 bp, Artificial Sequence
  Description of Artificial Sequence: Synthetic oligonucleotide
atctacatctgggcaccattggctgggacttgtggtgtccttctcctatcactggttatcacc

SEQ ID NO: 87 - protein, 21 aa, Artificial Sequence
  Description of Artificial Sequence: Synthetic peptide
IYIWAPLAGTCGVLLLSLVIT

(19) United States
(12) Patent Application Publication
(10) Pub. No.: US 2023/0183371 A1
(43) Pub. Date: Jun. 15, 2023

(54) ANTI-CD79B ANTIBODIES AND CHIMERIC ANTIGEN RECEPTORS AND METHODS OF USE THEREOF

(71) Applicant: Board of Regents, The University of Texas System, Austin, TX (US)

(72) Inventors: Fuliang CHU, Houston, TX (US); Sattva S. NEELAPU, Houston, TX (US); JingJing CAO, Houston, TX (US); Jingwei LIU, Houston, TX (US)

(73) Assignee: Board of Regents, The University of Texas System, Austin, TX (US)

(21) Appl. No.: 17/997,244

(22) PCT Filed: Apr. 30, 2021
(86) PCT No.: PCT/US2021/070497

§ 371 Date: Oct. 26, 2022

Related U.S. Application Data
(60) Provisional application No. 63/018,266, filed on Apr. 30, 2020.

(51) Int. Cl. C07K 16/28; A61P 35/00; C12N 5/0783; C12N 15/86

(52) U.S. Cl. CPC C07K 16/2896; A61P 35/00; C12N 5/0636; C12N 15/86; C07K 2317/565; C07K 2317/24; C07K 2319/02; C07K 2319/03; C12N 2740/15041; C12N 2310/20

(57) ABSTRACT

Provided herein are CD79b antibodies and CD79b-specific chimeric antigen receptors (CARs). Further provided herein are immune cells expressing the CD79b-specific CARs and methods of treating cancer by administering the CD79b-specific CAR immune cells.

This application claims the benefit of U.S. Provisional Application No. 63/018,266, filed Apr. 30, 2020, the entirety of which is incorporated herein by reference.

### BACKGROUND

### 1. Field

The present disclosure relates generally to the fields of immunology, cell biology, molecular biology, and medicine. More particularly, it concerns CD79b antibodies and related compositions, including at least chimeric antigen receptors and methods of use thereof.

### 2. Description of Related Art

Chimeric antigen receptor (CAR) T cells targeting CD19 are highly effective in B cell malignancies. Recently, two anti-CD19 CAR T-cell therapy products were approved by the US FDA for relapsed or refractory B cell acute lymphoblastic leukemia (ALL) and/or large B cell lymphoma. In pivotal trials, durable remissions lasting more than 1 year have been observed in ˜40-50% of these patients. However, relapse or progression occurs in ˜50-60% and a major cause of resistance appears to be because of CD19 antigen loss. Thus, there is an urgent need to develop CAR T cell therapies against novel targets to further improve outcomes in these patients.

### SUMMARY

The present disclosure concerns methods and compositions related to particular antibodies. The antibodies may be utilized in immunotherapies of any kind and for any medical application in which targeting of CD79b is therapeutic. In some embodiments, the present disclosure provides isolated monoclonal antibodies, wherein the antibodies specifically bind to CD79b and comprise:

### (I):

(a) a first V_{H} CDR comprising SEQ ID NO: 1;
(b) a second V_{H} CDR comprising SEQ ID NO: 2;
(c) a third V_{H} CDR comprising SEQ ID NO: 3;
(d) a first V_{L} CDR comprising SEQ ID NO: 4;
(e) a second V_{L} CDR comprising SEQ ID NO: 5; and
(f) a third V_{L} CDR comprising SEQ ID NO: 6;

### (II):

(a) a first V_{H} CDR comprising SEQ ID NO: 11;
(b) a second V_{H} CDR comprising SEQ ID NO: 12;
(c) a third V_{H} CDR comprising SEQ ID NO: 13;
(d) a first V_{L} CDR comprising SEQ ID NO: 14;
(e) a second V_{L} CDR comprising SEQ ID NO: 15; and
(f) a third V_{L} CDR comprising SEQ ID NO: 16; or

### (III):

(a) a first V_{H} CDR comprising SEQ ID NO: 21;
(b) a second V_{H} CDR comprising SEQ ID NO: 22;
(c) a third V_{H} CDR comprising SEQ ID NO: 23;
(d) a first V_{L} CDR comprising SEQ ID NO: 24;
(e) a second V_{L} CDR comprising SEQ ID NO: 25; and
(f) a third V_{L} CDR comprising SEQ ID NO: 26.

In some aspects, the antibody comprises:

(a) a first V_{H} CDR comprising SEQ ID NO: 1;
(b) a second V_{H} CDR comprising SEQ ID NO: 2;
(c) a third V_{H} CDR comprising SEQ ID NO: 3;
(d) a first V_{L} CDR comprising SEQ ID NO: 4;
(e) a second V_{L} CDR comprising SEQ ID NO: 5; and
(f) a third V_{L} CDR comprising SEQ ID NO: 6;

In further aspects, the antibody comprises a V_{H} domain at least about 80% identical to the V_{H} domain of SEQ ID NO: 7 and a V_{L} domain at least about 80% identical to the V_{L} domain of SEQ ID NO: 9. In other aspects, the antibody comprises a V_{H} domain identical to the V_{H} domain of SEQ ID NO: 7 and a V_{L} domain identical to the V_{L} domain of SEQ ID NO: 9.

In some aspects, the antibody comprises:

(a) a first VH CDR comprising SEQ ID NO: 11;

(b) a second VH CDR comprising SEQ ID NO: 12;

(c) a third VH CDR comprising SEQ ID NO: 13;

(d) a first VL CDR comprising SEQ ID NO: 14;

(e) a second VL CDR comprising SEQ ID NO: 15; and

(f) a third VL CDR comprising SEQ ID NO: 16.

In further aspects, the antibody comprises a V_{H} domain at least about 80% identical to the V_{H} domain of SEQ ID NO: 17 and a V_{L} domain at least about 80% identical to the V_{L} domain of SEQ ID NO: 19. In other aspects, the antibody comprises a V_{H} domain identical to the V_{H} domain of SEQ ID NO: 17 and a V_{L} domain identical to the V_{L} domain SEQ ID NO: 19.

In some aspects, the antibody comprises:

(a) a first V_{H} CDR comprising SEQ ID NO: 21;
(b) a second V_{H} CDR comprising SEQ ID NO: 22;
(c) a third V_{H} CDR comprising SEQ ID NO: 23;
(d) a first V_{L} CDR comprising SEQ ID NO: 24;
(e) a second V_{L} CDR comprising SEQ ID NO: 25; and
(f) a third V_{L} CDR comprising SEQ ID NO: 26.

In further aspects, the antibody comprises a V_{H} domain at least about 80% identical to the V_{H} domain of SEQ ID NO: 27 and a V_{L} domain at least about 80% identical to the V_{L} domain of SEQ ID NO: 29. In other aspects, the antibody comprises a V_{H} domain identical to the V_{H} domain of SEQ ID NO: 27 and a V_{L} domain identical to the V_{L} domain SEQ ID NO: 29.

In some aspects, the antibody is recombinant. In some aspects, the antibody is an IgG, IgM, IgA or an antigen binding fragment thereof. In some aspects, the antibody is a Fab′, a F(ab′)2, a F(ab′)3, a monovalent scFv, a bivalent scFv, or a single domain antibody. In some aspects, the antibody is a human, humanized antibody or de-immunized antibody. In some aspects, the antibody is conjugated to an imaging agent, a chemotherapeutic agent, a toxin or a radionuclide.

In other embodiments, the present disclosure provides compositions comprising an antibody of the present disclosure in a pharmaceutically acceptable carrier.

In still other embodiments, the present disclosure provides isolated polynucleotide molecules comprising a nucleic acid sequence encoding an antibody of the present disclosure.

In yet other embodiments, the present disclosure provides recombinant polypeptides comprising an antibody V_{H} domain comprising CDRs 1-3 of the V_{H} domain of Clone T26 (SEQ ID NOs: 1, 2, and 3) and CDRs 1-3 of the V_{L} domain of Clone T26 (SEQ ID NOs: 4, 5, and 6).

In other embodiments, the present disclosure provides recombinant polypeptide comprising an antibody V_{H} domain comprising CDRs 1-3 of the V_{H} domain of Clone 5B (SEQ ID NOs: 11, 12, and 13) and CDRs 1-3 of the V_{L} domain of Clone 5B (SEQ ID NOs: 14, 15, and 16).

In still other embodiments, the present disclosure provides recombinant polypeptides comprising an antibody V_{H} domain comprising CDRs 1-3 of the V_{H} domain of Clone 28B (SEQ ID NOs: 21, 22, and 23) and CDRs 1-3 of the V_{L} domain of Clone 28B (SEQ ID NOs: 24, 25, and 26).

In yet other embodiments, the present disclosure provides isolated polynucleotide molecules comprising a nucleic acid sequence encoding a polypeptide of the present disclosure.

In other embodiments, the present disclosure provides host cells comprising one or more polynucleotide molecule(s) encoding an antibody of the present disclosure or a recombinant polypeptide of the present disclosure. In some aspects, the host cell is a mammalian cell, a yeast cell, a bacterial cell, a ciliate cell or an insect cell.

In still other embodiments, the present disclosure provides methods for treating a subject having a cancer comprising administering an effective amount of an antibody of the present disclosure to the subject. In some aspects, the cancer is B cell malignancy. In some aspects, the antibody is in a pharmaceutically acceptable composition. In some aspects, the antibody is administered systemically. In some aspects, the antibody is administered intravenously, intradermally, intratumorally, intramuscularly, intraperitoneally, subcutaneously, or locally. In some aspects, the methods further comprise administering at least a second anticancer therapy to the subject. In further aspects, the second anticancer therapy is a surgical therapy, chemotherapy, radiation therapy, cryotherapy, hormonal therapy, immunotherapy or cytokine therapy. In some aspects, the second anticancer therapy comprises an adoptive T-cell therapy.

In yet other embodiments, the present disclosure provides engineered CD79b CAR or TCR having an antigen binding domain comprising:

### (I):

(a) a first V_{H} CDR comprising SEQ ID NO: 1;
(b) a second V_{H} CDR comprising SEQ ID NO: 2;
(c) a third V_{H} CDR comprising SEQ ID NO: 3;
(d) a first V_{L} CDR comprising SEQ ID NO: 4;
(e) a second V_{L} CDR comprising SEQ ID NO: 5; and
(f) a third V_{L} CDR comprising SEQ ID NO: 6;

### (II):

(a) a first V_{H} CDR comprising SEQ ID NO: 11;
(b) a second V_{H} CDR comprising SEQ ID NO: 12;
(c) a third V_{H} CDR comprising SEQ ID NO: 13;
(d) a first V_{L} CDR comprising SEQ ID NO: 14;
(e) a second V_{L} CDR comprising SEQ ID NO: 15; and
(f) a third V_{L} CDR comprising SEQ ID NO: 16; or

### (III):

(a) a first V_{H} CDR comprising SEQ ID NO: 21;
(b) a second V_{H} CDR comprising SEQ ID NO: 22;
(c) a third V_{H} CDR comprising SEQ ID NO: 23;
(d) a first V_{L} CDR comprising SEQ ID NO: 24;
(e) a second V_{L} CDR comprising SEQ ID NO: 25; and
(f) a third V_{L} CDR comprising SEQ ID NO: 26.

In some aspects, the antigen-binding domain comprises:

(a) a first V_{H} CDR comprising SEQ ID NO: 1;
(b) a second V_{H} CDR comprising SEQ ID NO: 2;
(c) a third V_{H} CDR comprising SEQ ID NO: 3;
(d) a first V_{L} CDR comprising SEQ ID NO: 4;
(e) a second V_{L} CDR comprising SEQ ID NO: 5; and
(f) a third V_{L} CDR comprising SEQ ID NO: 6.

In further aspects, the antigen-binding domain comprises a V_{H} domain at least about 80% identical to the V_{H} domain of SEQ ID NO: 7 and a V_{L} domain at least about 80% identical to the V_{L} domain of SEQ ID NO: 9. In some aspects, the antigen-binding domain comprises a V_{H} domain identical to the V_{H} domain of SEQ ID NO: 7 and a V_{L} domain identical to the V_{L} domain of SEQ ID NO: 9.

In some aspects, the antibody comprises:

(a) a first V_{H} CDR comprising SEQ ID NO: 11;
(b) a second V_{H} CDR comprising SEQ ID NO: 12;
(c) a third V_{H} CDR comprising SEQ ID NO: 13;
(d) a first V_{L} CDR comprising SEQ ID NO: 14;
(e) a second V_{L} CDR comprising SEQ ID NO: 15; and
(f) a third V_{L} CDR comprising SEQ ID NO: 16.

In further aspects, the antigen-binding domain comprises a V_{H} domain at least about 80% identical to the V_{H} domain of SEQ ID NO: 17 and a V_{L} domain at least about 80% identical to the V_{L} domain of SEQ ID NO: 19. In some aspects, the antigen-binding domain comprises a V_{H} domain identical to the V_{H} domain of SEQ ID NO: 17 and a V_{L} domain identical to the V_{L} domain SEQ ID NO: 19.

In some aspects, the antigen-binding domain comprises:

(a) a first V_{H} CDR comprising SEQ ID NO: 21;
(b) a second V_{H} CDR comprising SEQ ID NO: 22;
(c) a third V_{H} CDR comprising SEQ ID NO: 23;
(d) a first V_{L} CDR comprising SEQ ID NO: 24;
(e) a second V_{L} CDR comprising SEQ ID NO: 25; and
(f) a third V_{L} CDR comprising SEQ ID NO: 26.

In further aspects, the antigen-binding domain comprises a V_{H} domain at least about 80% identical to the V_{H} domain of SEQ ID NO: 27 and a V_{L} domain at least about 80% identical to the V_{L} domain of SEQ ID NO: 29. In some aspects, the antigen-binding domain comprises a V_{H} domain identical to the V_{H} domain of SEQ ID NO: 27 and a V_{L} domain identical to the V_{L} domain SEQ ID NO: 29. In some aspects, the CAR comprises one or more signaling domains CD3ξ, CD28, OX40/CD134, 4-1BB/CD137, or a combination thereof. In some aspects, the CAR comprises CD3ζ and CD28 signaling domains. In some aspects, the CAR comprises CD3ζ and 4-1BB signaling domains. In some aspects, the CAR comprises CD3ζ and OX-40 signaling domains. In some aspects, the CAR or TCR is encoded by a viral vector. In further aspects, the viral vector is a lentiviral vector.

In some aspects, the antigen-binding domain comprises a V_{H} domain linked to a V_{L} domain by a linker. In further aspects, the linker is Linker 1 (SEQ ID NOs: 44 or 45), Linker 2 (SEQ ID NOs:46 or 47), Linker 3 (SEQ ID NOs:48 or 49), or Linker 4 (SEQ ID NOs:50 or 51). In some aspects, the CAR comprises V_{L}-Linker1-V_{H}, V_{L}-Linker2-V_{H}, V_{L}-Linker3-V_{H}, V_{L}-Linker4-V_{H}, V_{H}-Linker1-V_{L}, V_{H}-Linker2-V_{L}, V_{H}-Linker3-V_{L}, or V_{H}-Linker4-V_{L}. In some aspects, the CAR or TCR comprises a hinge. In further aspects, the hinge is CD8 Hinge 1 (SEQ ID NOs:52 or 53), CD8 Hinge 2 (SEQ ID NOs:54 or 55), CD8 Hinge 3 (SEQ ID NOs:56 or 57), CD28 Hinge (SEQ ID NOs:58 or 59), IgG4 Hinge (SEQ ID NOs:60 or 61), IgG4 CH2 (SEQ ID NOs:62 or 63), IgG4 CH2CH3 (SEQ ID NOs:64 or 65), or IgG4 CH1CH2CH3 (SEQ ID NOs:66 or 67). In some aspects, the CAR comprises a transmembrane domain. In further aspects, the transmembrane domain is CD8 TM1 (SEQ ID NOs:68 or 69), CD8 TM2 (SEQ ID NOs:70 or 71), or CD28 TM (SEQ ID NOs:72 or 73).

In some aspects, the methods further comprise a transduction marker and/or safety switch. In further aspects, the transduction marker is enhanced green fluorescent protein (eGFP). In still further aspects, the eGFP has an amino acid sequence of SEQ ID NO:83. In some aspects, the transduction marker and/or safety switch is truncated epidermal growth factor (EGFR). In further aspects, the EGFR has an amino acid sequence of SEQ ID NO:41. In some aspects, the transduction marker and/or safety switch is linked to the CAR by a cleavage peptide. In further aspects, the cleavage peptide is a 2A peptide. In still further aspects, the 2A peptide if a T2A peptide. In yet further aspects, the T2A peptide has an amino acid sequence of SEQ ID NO:85. In some aspects, the CAR further comprises a second antigen binding domain. In further aspects, the second antigen binding domain is a CD19, CD20, or CD22 antigen binding domain.

In other embodiments, the present disclosure provides expression vectors encoding the CAR or TCR of the present disclosure.

In still other embodiments, the present disclosure provides host cells engineered to express a CD79b CAR or a CD79b TCR. In some aspects, the cell is engineered to express a CAR of the present disclosure. In some aspects, the host cell is an immune cell. In further aspects, the immune cell is a T cell. In still further aspects, the T cell is a primary human T cell or a TIL. In other aspects, the T cell is a CD4+ T cell or CD8+ T cell. In some aspects, the primary human T cell is obtained from a healthy donor. In some aspects, the T cell is autologous. In some aspects, the T cell is allogeneic. In some aspects, the cell is engineered using a CRISPR or transposase system.

In yet other embodiments, the present disclosure provides pharmaceutical compositions comprising CD79b targeted T cells and a pharmaceutical carrier, wherein the CD79b targeted T cells are engineered to express a CAR or TCR of the present disclosure.

In other embodiments, the present disclosure provides compositions comprising an effective amount of CD79b targeted T cells for the treatment of cancer in a subject, wherein the CD79b targeted T cells are engineered to express a CAR or TCR of the present disclosure.

In yet other embodiments, the present disclosure provides use of a composition comprising an effective amount of CD79b targeted T cells for the treatment of cancer in a subject, wherein the CD79b targeted T cells are engineered to express a CAR or TCR of the present disclosure.

In other embodiments, the present disclosure provides methods for treating cancer in a subject comprising administering an effective amount of CD79b-targeted T cells to the subject, wherein the CD79b targeted T cells are engineered to express a CAR or TCR of the present disclosure. In further aspects, the cancer is a B cell malignancy. In still further aspects, the B cell malignancy is B cell acute lymphoblastic leukemia (ALL), diffuse, large B cell lymphoma, follicular lymphoma, marginal zone lymphoma, lymphoplasmacytic lymphoma, Burkitt lymphoma, or chronic lymphocytic leukemia. In some aspects, the subject has been previously administered a CD19 CAR therapy. In some aspects, the subject is resistant to CD19 CAR therapy. In further aspects, the subject has CD19 antigen loss. In still further aspects, the subject has relapsed with a CD19-negative tumor. In some aspects, the CD79b targeted T cells are administered intravenously, intradermally, intratumorally, intramuscularly, intraperitoneally, subcutaneously, or locally. In some aspects, the CD79b targeted T cells are administered intravenously. In some aspects, the methods further comprise administering at least a second anticancer therapy to the subject. In further aspects, the second anticancer therapy is a surgical therapy, chemotherapy, radiation therapy, cryotherapy, hormonal therapy, immunotherapy or cytokine therapy. In some aspects, the cancer is a CD79b-expressing cancer.

In certain aspects, the CAR further comprises a second antigen binding domain. In some aspects, the second antigen binding domain is a CD19, CD20, or CD22 antigen binding domain.

In another embodiment, there is provided an expression vector encoding a CD79b CAR of the present embodiments.

Further provided herein is a host cell engineered to express a CD79b CAR, such as a CD79b of the present embodiments. In some aspects, the host cell is an immune cell, such as a T cell. In some aspects, the T cell is a primary human T cell. In certain aspects, the T cell is a CD4+ T cell or CD8+ T cell. In some aspects, the primary human T cell is obtained from a healthy donor. The T cell may be autologous or allogeneic.

Also provided herein is a pharmaceutical composition comprising CD79b CAR T cells, such as CAR T cells of the present embodiments, and a pharmaceutical carrier. Further provided herein is a composition comprising an effective amount of CD79b CAR T cells, such as CAR T cells of the present embodiments, for the treatment of cancer in a subject. In another embodiments, there is provided the use of a composition comprising an effective amount of CD79b CAR T cells, such as CAR T cells of the present embodiments, for the treatment of cancer in a subject.

In a further embodiment, there is provided a method for treating cancer in a subject comprising administering an effective amount of CD79b CAR T cells, such as CAR T cells of the present embodiments, to the subject. In some aspects, the cancer is a B cell malignancy, such as B cell acute lymphoblastic leukemia (ALL), diffuse, large B cell lymphoma, follicular lymphoma, marginal zone lymphoma, lymphoplasmacytic lymphoma, Burkitt lymphoma, or chronic lymphocytic leukemia. In certain aspects, the cancer is a CD79b-expressing cancer.

In some aspects, the subject has been previously administered a CD19 CAR therapy. In certain aspects, the subject is resistant to CD19 CAR therapy, such as due to CD19 antigen loss. In certain aspects, the subject has relapsed with a CD19-negative tumor.

In certain aspects, the CD79b CAR T cells are administered intravenously, intradermally, intratumorally, intramuscularly, intraperitoneally, subcutaneously, or locally. In additional aspects, the method further comprises administering at least a second anticancer therapy to the subject. In some aspects, the second anticancer therapy is a surgical therapy, chemotherapy, radiation therapy, cryotherapy, hormonal therapy, immunotherapy or cytokine therapy.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1D: (FIG. 1A) CD79b expression in cell lines. (FIG. 1B) CD79b expression in human tissues. (FIG. 1C) CD79b expression in leukemias. (FIG. 1D) CD79b expression in lymphomas.

FIGS. 2A-2D: (FIG. 2A) Flow cytometry analysis of cells transduced with CD79b. (FIG. 2B) Binding affinity of CD79b monoclonal antibodies. (FIG. 2C) Characterization of CD79b monoclonal antibodies. (FIG. 2D) Clone 14 staining of lymphoma cell lines.

FIGS. 3A-3D: (FIG. 3A) Schematic depicting constructs for CD79b CARs. (FIG. 3B) Flow cytometry analysis of CD79b CAR and CD19 CAR. (FIG. 3C) Percent cytotoxicity of CD79b CAR and CD19 CAR with untransduced T cells as control. (FIG. 3D) Flow cytometry of CD79b CAR and CD19 CAR with untransduced T cells as control.

FIGS. 4A-4D: (FIG. 4A) T cells co-cultured with CD79b CAR and CD19 Exon 2Δ splice variant. (FIG. 4B) Flow cytometry analysis of efficacy of CAR at Effector:Target ratio of 5:1 incubated for 4 days. (FIG. 4C) Absolute cell count of Daudi cells with CD79b CAR. (FIG. 4D) Absolute cell count of CD19 knockdown cells with CD79b CAR.

FIGS. 5A-5C: (FIG. 5A) Schematic of pre-clinical study. (FIG. 5B) Bioluminescence images of mice during study. (FIG. 5C) Percent survival of mice during study.

FIGS. 6A-6E: (FIG. 6A) CD79b CAR constructs of certain embodiments. (FIGS. 6B-6C) Graphs and histograms show Anti-CD79b CAR T cells exhibit cytotoxicity in vitro against Daudi lymphoma cells. (FIGS. 6D-6E) Graphs and imaging show that anti-CD79b CAR T cells exhibit in vivo efficacy against Daudi lymphoma xenografts.

FIG. 7: Binding of anti-CD79b antibodies (clones 5B and 28B) to human CD79b

FIGS. 8A-8B: (FIG. 8A) Domain map of anti-CD79b CARs used in embodiments herein. (FIG. 8B) Map of CAR constructs in a lentiviral vector (pLVEG) containing an EF1α promoter.

FIGS. 9A-9D: (FIG. 9A) Transduction of CARs in NFAT reporter cells. (FIG. 9B) Luciferase activity in transduced NFAT reporter cells co-cultured with antibodies or Dadui Burkitt cells. (FIGS. 9C-9D) Luciferase activity in transduced NFAT reporter cells co-cultured with SUDHL6.

FIGS. 10A-10B: (FIG. 10A) Representative transduction efficiency of CARs in T cells. (FIG. 10B) Phosphorylation of CD3ζ and ERK1/2 in T cells expressing or not expressing a CAR.

FIGS. 11A-11B: (FIGS. 11A-11B) Proliferation of T cells expressing or not expressing a CAR.

FIG. 12: Cytokine expression of T cells expressing or not expressing a CAR

FIGS. 13A-13B: (FIGS. 13A-13B) Degranulation of T cells expressing or not expressing a CAR in response to lymphoma cells

FIGS. 14A-14B: (FIG. 14A) Cytotoxic activity of T cells expressing or not expressing a CAR to lymphoma cells. (FIG. 14B) Lysis of SUDHL6 cells by CAR T cells.

FIGS. 15A-15B: (FIG. 15A) Bioluminescence imaging of tumor burden in mice treated with T cells expressing or not expressing a CAR. (FIG. 15B) Probability of survival in a mouse cancer model treated with cells expressing or not expressing a CAR.

### DESCRIPTION OF ILLUSTRATIVE EMBODIMENTS

CD79b is a pan B cell linage marker and an important component of the B cell receptor complex. CD79b is broadly expressed in normal B cells and B cell malignancies and its expression is usually retained in CD19 negative tumors relapsing after CD19-specific CAR T cell therapy. Accordingly, in certain embodiments, the present disclosure provides CD79b monoclonal antibodies and CD79b-specific CARs, such as for CD79b-CAR T cells.

The present studies demonstrated the efficacy of the present CD79b-specific CAR T cell product in in vitro and in vivo models. Three murine monoclonal antibodies were developed against human CD79b by hybridoma technology and it was demonstrated that they bind specifically to recombinant human CD79b, have high affinity (Kd range of 1.44-17.8 nM), and stain multiple lymphoma cell lines. Next, the variable regions of the heavy and light chains of the CD79b antibodies were cloned, and lentiviral constructs were developed for the anti-CD79b CARs with CD3ζ [symbol] and CD28/4-1BB costimulatory domains. It was demonstrated that the anti-CD79b CAR constructs can be transduced into primary CD4₊ and CD8₊ T cells from healthy donors using lentivirus to more than 70% transduction efficiency.

It was observed that the anti-CD79b CAR T cells but not untransduced T cells demonstrated significant cytotoxic activity that was comparable to control anti-CD19 CART cells against Daudi Burkitt lymphoma and Mino mantle cell lymphoma cell lines. More importantly, anti-CD79b but not anti-CD19 CAR T cells lysed CD19-CD79b₊ lymphoma cells. Significant degranulation was also observed in both CD4⁺ and CD8⁺ anti-CD79b CAR T cells when they were co-cultured with lymphoma cells. The efficacy of anti-CD79b CAR T cells was also examined in vivo against Mino lymphoma xenograft model in NSG mice. Luciferase-labeled Mino mantle cell lymphoma cells were injected IV into NSG mice at 2×10⁶ tumor cells/mouse. After 18 days, mice were treated with untransduced primary T cells, anti-CD19 CAR T cells, or anti-CD79b CAR T cells via tail vein at 10×10⁶ T cells/mouse. Bioluminescence imaging was used to assess tumor burden. The results showed progressive tumor growth in mice treated with untransduced T cells. While in mice treated with anti-CD19- and anti-CD79b CAR T cells tumor growth was inhibited and survival was improved. Thus, these results showed the efficacy of this novel anti-CD79b CAR T cell therapy in patients with B cell malignancies which could be a novel strategy to overcome resistance due to CD19 loss after CD19-specific CAR T-cell therapy.

In some aspects, the present anti-CD79b CAR construct is encoded by a lentiviral vector. The vector may be transduced into immune cells, such as T cells. The construct may comprise CD28, CD3ζ, and/or 4-1BB signaling domains. The construct can comprise a transduction marker, such as eGFP or a truncated EGFR domain. The transduction marker may be linked to the CAR by a cleavage peptide, such as a 2A peptide.

Further provided herein are methods of treating cancer by administering the CD79b-specific CAR immune cells, such as T cells, provided herein. The cancer may be a B cell malignancy, such as B cell acute lymphoblastic leukemia (ALL), diffuse large B cell lymphoma, follicular lymphoma, marginal zone lymphoma, lymphoplasmacytic lymphoma, Burkitt lymphoma, or chronic lymphocytic leukemia that express CD79b. The present therapy may be used to treat subjects with a B cell malignancy who has relapsed with CD19 negative tumors after anti-CD19-CAR T cell therapy.

### II. DEFINITIONS

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more. The terms “about”, “substantially” and “approximately” mean, in general, the stated value plus or minus 5%.

“Treating” or treatment of a disease or condition refers to executing a protocol, which may include administering one or more drugs to a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

“Subject” and “patient” refer to either a human or non-human, such as primates, mammals, and vertebrates. In particular embodiments, the subject is a human.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

### III. CD79B ANTIBODIES

In certain embodiments, an antibody or a fragment thereof that binds to at least a portion of CD79b and inhibits CD79b activity of any kind, including at least signaling, are contemplated. As used herein, the term “antibody” is intended to refer broadly to any immunologic binding agent, such as IgG, IgM, IgA, IgD, IgE, and genetically modified IgG as well as polypeptides comprising antibody CDR domains that retain antigen binding activity. The antibody may be selected from the group consisting of a chimeric antibody, an affinity matured antibody, a polyclonal antibody, a monoclonal antibody, a humanized antibody, a human antibody, or an antigen-binding antibody fragment or a natural or synthetic ligand. In specific cases, the anti-CD79b antibody is a monoclonal antibody or a humanized antibody.

Thus, by known means and as described herein, polyclonal or monoclonal antibodies, antibody fragments, and binding domains and CDRs (including engineered forms of any of the foregoing) may be created that are specific to CD79b, one or more of its respective epitopes, or conjugates of any of the foregoing, whether such antigens or epitopes are isolated from natural sources or are synthetic derivatives or variants of the natural compounds.

Examples of antibody fragments suitable for the present embodiments include, without limitation: (i) the Fab fragment, consisting of V_{L}, V_{H}, C_{L}, and C_{H1} domains; (ii) the “Fd” fragment consisting of the V_{H} and C_{H1} domains; (iii) the “Fv” fragment consisting of the V_{L} and V_{H} domains of a single antibody; (iv) the “dAb” fragment, which consists of a V_{H} domain; (v) isolated CDR regions; (vi) F(ab′)2 fragments, a bivalent fragment comprising two linked Fab fragments; (vii) single chain Fv molecules (“scFv”), wherein a V_{H} domain and a V_{L} domain are linked by a peptide linker that allows the two domains to associate to form a binding domain; (viii) bi-specific single chain Fv dimers (see U.S. Pat. No. 5,091,513); and (ix) diabodies, multivalent or multispecific fragments constructed by gene fusion (US Patent App. Pub. 20050214860). Fv, scFv, or diabody molecules may be stabilized by the incorporation of disulphide bridges linking the V_{H} and V_{L} domains. Minibodies comprising a scFv joined to a CH3 domain may also be made.

Antibody-like binding peptidomimetics are also contemplated in embodiments. Liu et al. (2003) describe “antibody like binding peptidomimetics” (ABiPs), which are peptides that act as pared-down antibodies and have certain advantages of longer serum half-life as well as less cumbersome synthesis methods.

Animals may be inoculated with an antigen, such as a CD79b extracellular domain (ECD) protein, in order to produce antibodies specific for CD79b. Frequently an antigen is bound or conjugated to another molecule to enhance the immune response. As used herein, a conjugate is any peptide, polypeptide, protein, or non-proteinaceous substance bound to an antigen that is used to elicit an immune response in an animal. Antibodies produced in an animal in response to antigen inoculation comprise a variety of non-identical molecules (polyclonal antibodies) made from a variety of individual antibody producing B lymphocytes. A polyclonal antibody is a mixed population of antibody species, each of which may recognize a different epitope on the same antigen. Given the correct conditions for polyclonal antibody production in an animal, most of the antibodies in the animal's serum will recognize the collective epitopes on the antigenic compound to which the animal has been immunized. This specificity is further enhanced by affinity purification to select only those antibodies that recognize the antigen or epitope of interest.

A monoclonal antibody is a single species of antibody wherein every antibody molecule recognizes the same epitope because all antibody producing cells are derived from a single B-lymphocyte cell line. The methods for generating monoclonal antibodies (MAbs) generally begin along the same lines as those for preparing polyclonal antibodies. In some embodiments, rodents such as mice and rats are used in generating monoclonal antibodies. In some embodiments, rabbit, sheep, or frog cells are used in generating monoclonal antibodies. The use of rats is well known and may provide certain advantages. Mice (e.g., BALB/c mice) are routinely used and generally give a high percentage of stable fusions.

Hybridoma technology involves the fusion of a single B lymphocyte from a mouse previously immunized with a CD79b antigen with an immortal myeloma cell (usually mouse myeloma). This technology provides a method to propagate a single antibody-producing cell for an indefinite number of generations, such that unlimited quantities of structurally identical antibodies having the same antigen or epitope specificity (monoclonal antibodies) may be produced.

Plasma B cells (CD45⁺CD5⁻CD19⁺) may be isolated from freshly prepared rabbit peripheral blood mononuclear cells of immunized rabbits and further selected for CD79b binding cells. After enrichment of antibody producing B cells, total RNA may be isolated and cDNA synthesized. DNA sequences of antibody variable regions from both heavy chains and light chains may be amplified, constructed into a phage display Fab expression vector, and transformed into E. coli. CD79b specific binding Fab may be selected out through multiple rounds enrichment panning and sequenced. Selected CD79b binding hits may be expressed as full-length IgG in rabbit and rabbit/human chimeric forms using a mammalian expression vector system in human embryonic kidney (HEK293) cells (Invitrogen) and purified using a protein G resin with a fast protein liquid chromatography (FPLC) separation unit.

In one embodiment, the antibody is a chimeric antibody, for example, an antibody comprising antigen binding sequences from a non-human donor grafted to a heterologous non-human, human, or humanized sequence (e.g., framework and/or constant domain sequences). Methods have been developed to replace light and heavy chain constant domains of the monoclonal antibody with analogous domains of human origin, leaving the variable regions of the foreign antibody intact. Alternatively, “fully human” monoclonal antibodies are produced in mice transgenic for human immunoglobulin genes. Methods have also been developed to convert variable domains of monoclonal antibodies to more human form by recombinantly constructing antibody variable domains having both rodent, for example, mouse, and human amino acid sequences. In “humanized” monoclonal antibodies, only the hypervariable CDR is derived from mouse monoclonal antibodies, and the framework and constant regions are derived from human amino acid sequences (see U.S. Pat. Nos. 5,091,513 and 6,881,557). It is thought that replacing amino acid sequences in the antibody that are characteristic of rodents with amino acid sequences found in the corresponding position of human antibodies will reduce the likelihood of adverse immune reaction during therapeutic use. A hybridoma or other cell producing an antibody may also be subject to genetic mutation or other changes, which may or may not alter the binding specificity of antibodies produced by the hybridoma.

Methods for producing polyclonal antibodies in various animal species, as well as for producing monoclonal antibodies of various types, including humanized, chimeric, and fully human, are well known in the art and highly predictable. For example, the following U.S. patents and patent applications provide enabling descriptions of such methods: U.S. Patent Application Nos. 2004/0126828 and 2002/0172677; and U.S. Pat. Nos. 3,817,837; 3,850,752; 3,939,350; 3,996,345; 4,196,265; 4,275,149; 4,277,437; 4,366,241; 4,469,797; 4,472,509; 4,606,855; 4,703,003; 4,742,159; 4,767,720; 4,816,567; 4,867,973; 4,938,948; 4,946,778; 5,021,236; 5,164,296; 5,196,066; 5,223,409; 5,403,484; 5,420,253; 5,565,332; 5,571,698; 5,627,052; 5,656,434; 5,770,376; 5,789,208; 5,821,337; 5,844,091; 5,858,657; 5,861,155; 5,871,907; 5,969,108; 6,054,297; 6,165,464; 6,365,157; 6,406,867; 6,709,659; 6,709,873; 6,753,407; 6,814,965; 6,849,259; 6,861,572; 6,875,434; and 6,891,024. All patents, patent application publications, and other publications cited herein and therein are hereby incorporated by reference in the present application.

Antibodies may be produced from any animal source, including birds and mammals. Preferably, the antibodies are ovine, murine (e.g., mouse and rat), rabbit, goat, guinea pig, camel, horse, or chicken. In addition, newer technology permits the development of and screening for human antibodies from human combinatorial antibody libraries. For example, bacteriophage antibody expression technology allows specific antibodies to be produced in the absence of animal immunization, as described in U.S. Pat. No. 6,946,546, which is incorporated herein by reference.

It is fully expected that antibodies to CD79b will have the ability to neutralize or counteract the effects of CD79b regardless of the animal species, monoclonal cell line, or other source of the antibody. Certain animal species may be less preferable for generating therapeutic antibodies because they may be more likely to cause allergic response due to activation of the complement system through the “Fc” portion of the antibody. However, whole antibodies may be enzymatically digested into “Fc” (complement binding) fragment, and into antibody fragments having the binding domain or CDR. Removal of the Fc portion reduces the likelihood that the antigen antibody fragment will elicit an undesirable immunological response, and thus, antibodies without Fc may be preferential for prophylactic or therapeutic treatments. As described above, antibodies may also be constructed so as to be chimeric or partially or fully human, so as to reduce or eliminate the adverse immunological consequences resulting from administering to an animal an antibody that has been produced in, or has sequences from, other species.

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

Proteins may be recombinant, or synthesized in vitro. Alternatively, a non-recombinant or recombinant protein may be isolated from bacteria. It is also contemplated that a bacteria containing such a variant may be implemented in compositions and methods. Consequently, a protein need not be isolated.

It is contemplated that in compositions there is between about 0.001 mg and about 10 mg of total polypeptide, peptide, and/or protein per ml. Thus, the concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein). Of this, about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% may be an antibody that binds CD79b.

An antibody or preferably an immunological portion of an antibody, can be chemically conjugated to, or expressed as, a fusion protein with other proteins. For purposes of this specification and the accompanying claims, all such fused proteins are included in the definition of antibodies or an immunological portion of an antibody.

Embodiments provide antibodies and antibody-like molecules against CD79b, polypeptides and peptides that are linked to at least one agent to form an antibody conjugate or payload. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules that have been attached to antibodies include toxins, therapeutic enzymes, antibiotics, radio-labeled nucleotides and the like. By contrast, a reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3-6-diphenylglycouril-3 attached to the antibody. Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate.

### IV. CELL THERAPY

Certain embodiments of the present disclosure concern obtaining and administering cells to a subject as an immunotherapy to target cancer cells. The cells may deliver antibody compositions encompassed herein but themselves may or may not be immune cells. In specific embodiments the cells are immune cells. Examples of cells include T cells (including αβ T cells or γδ T cells), Natural Killer (NK) cells, invariant NKT (iNKT) cells, B cells, macrophages, stem cells of any kind (including MSCs or induced pluripotent stem cells), or dendritic cells.

Several basic approaches for the derivation, activation and expansion of functional anti-tumor effector T cells have been described in the last two decades. These include: autologous cells, such as tumor-infiltrating lymphocytes (TILs); T cells activated ex-vivo using autologous DCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TCR); and non-tumor-specific autologous or allogeneic cells genetically reprogrammed or “redirected” to express tumor-reactive TCR or chimeric TCR molecules displaying antibody-like tumor recognition capacity known as “T-bodies”. These approaches have given rise to numerous protocols for T cell preparation and immunization which can be used in the methods of the present disclosure.

A. T Cell Preparation

In some embodiments, the T cells are derived from the blood, bone marrow, lymph, or lymphoid organs. In some aspects, the cells are human cells. The cells typically are primary cells, such as those isolated directly from a subject and/or isolated from a subject and frozen. In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells (e.g., CD4⁺ and/or CD8⁺ T cells) are naive T (T_{N}) cells, effector T cells (T_{EFF}), memory T cells and sub-types thereof, such as stem cell memory T (TSC_{M}), central memory T (T_{CM}), effector memory T (T_{EM}), or terminally differentiated effector memory T (T_{TEMRA}) cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, one or more of the T cell populations is enriched for or depleted of cells that are positive for a specific marker, such as surface markers, or that are negative for a specific marker. In some cases, such markers are those that are absent or expressed at relatively low levels on certain populations of T cells (e.g., non-memory cells) but are present or expressed at relatively higher levels on certain other populations of T cells (e.g., memory cells). In one embodiment, the cells (e.g., CD8⁺ cells or CD3⁺ cells) are enriched for (i.e., positively selected for) cells that are positive or expressing high surface levels of CD45RO, CCR7, CD28, CD27, CD44, CD127, and/or CD62L and/or depleted of (e.g., negatively selected for) cells that are positive for or express high surface levels of CD45RA. In some embodiments, cells are enriched for or depleted of cells positive or expressing high surface levels of CD122, CD95, CD25, CD27, and/or IL7-Ra (CD127). In some examples, CD8⁺ T cells are enriched for cells positive for CD45RO (or negative for CD45RA) and for CD62L.

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long-term survival, expansion, and/or engraftment following administration, which in some aspects is particularly robust in such sub-populations. In some embodiments, combining T_{CM}-enriched CD8⁺ T cells and CD4⁺ T cells further enhances efficacy.

In some embodiments, the T cells are autologous T cells. In this method, tumor samples are obtained from patients and a single cell suspension is obtained. The single cell suspension can be obtained in any suitable manner, e.g., mechanically (disaggregating the tumor using, e.g., a gentleMACS™ Dissociator, Miltenyi Biotec, Auburn, Calif.) or enzymatically (e.g., collagenase or DNase). Single-cell suspensions of tumor enzymatic digests are cultured in interleukin-2 (IL-2). The cells are cultured until confluence (e.g., about 2×10⁶ lymphocytes), e.g., from about 5 to about 21 days, preferably from about 10 to about 14 days. For example, the cells may be cultured from 5 days, 5.5 days, or 5.8 days to 21 days, 21.5 days, or 21.8 days, such as from 10 days, 10.5 days, or 10.8 days to 14 days, 14.5 days, or 14.8 days.

The cultured T cells can be pooled and rapidly expanded. Rapid expansion provides an increase in the number of antigen-specific T-cells of at least about 50-fold (e.g., 50-, 60-, 70-, 80-, 90-, or 100-fold, or greater) over a period of about 10 to about 14 days, preferably about 14 days. More preferably, rapid expansion provides an increase of at least about 200-fold (e.g., 200-, 300-, 400-, 500-, 600-, 700-, 800-, 900-, or greater) over a period of about 10 to about 14 days, preferably about 14 days.

Expansion can be accomplished by any of a number of methods as are known in the art. For example, T cells can be rapidly expanded using non-specific T-cell receptor stimulation in the presence of feeder lymphocytes and either interleukin-2 (IL-2) or interleukin-15 (IL-15), with IL-2 being preferred. The non-specific T-cell receptor stimulus can include around 30 ng/ml of OKT3, a mouse monoclonal anti-CD3 antibody (available from Ortho-McNeil®, Raritan, N.J.). Alternatively, T cells can be rapidly expanded by stimulation of peripheral blood mononuclear cells (PBMC) in vitro with one or more antigens (including antigenic portions thereof, such as epitope(s), or a cell) of the cancer, which can be optionally expressed from a vector, such as an human leukocyte antigen A2 (HLA-A2) binding peptide, in the presence of a T-cell growth factor, such as 300 IU/ml IL-2 or IL-15, with IL-2 being preferred. The in vitro-induced T-cells are rapidly expanded by re-stimulation with the same antigen(s) of the cancer pulsed onto HLA-A2-expressing antigen-presenting cells. Alternatively, the T-cells can be re-stimulated with irradiated, autologous lymphocytes or with irradiated HLA-A2+ allogeneic lymphocytes and IL-2, for example.

The autologous T-cells can be modified to express a T-cell growth factor that promotes the growth and activation of the autologous T-cells. Suitable T-cell growth factors include, for example, interleukin (IL)-2, IL-7, IL-15, and IL-12. Suitable methods of modification are known in the art. See, for instance, Sambrook et al., Molecular Cloning: A Laboratory Manual, 3^{rd} ed., Cold Spring Harbor Press, Cold Spring Harbor, N.Y. 2001; and Ausubel et al., Current Protocols in Molecular Biology, Greene Publishing Associates and John Wiley & Sons, N Y, 1994. In particular aspects, modified autologous T-cells express the T-cell growth factor at high levels. T-cell growth factor coding sequences, such as that of IL-12, are readily available in the art, as are promoters, the operable linkage of which to a T-cell growth factor coding sequence promote high-level expression.

B. Genetically Engineered Antigen Receptors

The cell can be genetically engineered to express engineered antigen receptors such as engineered TCRs or chimeric antigen receptors (CARs). For example, the autologous T-cells are modified to express a T cell receptor (TCR) having antigenic specificity for a cancer antigen, such as CD79b. Suitable TCRs include, for example, those with antigenic specificity for a melanoma antigen, e.g., gp100 or MART-1. Suitable methods of modification are known in the art. See, for instance, Sambrook and Ausubel, supra. For example, the T cells may be transduced to express a TCR having antigenic specificity for a cancer antigen using transduction techniques described in Heemskerk et al. Hum Gene Ther. 19:496-510 (2008) and Johnson et al. Blood 114:535-46 (2009).

In some embodiments, the T cells comprise one or more nucleic acids introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g., chimeric).

In some embodiments, the CAR contains an extracellular antigen-recognition domain that specifically binds to CD79b. In some embodiments, the antigen is a protein expressed on the surface of cells. In some embodiments, the CAR is a TCR-like CAR and the antigen is a processed peptide antigen, such as a peptide antigen of an intracellular protein, which, like a TCR, is recognized on the cell surface in the context of a major histocompatibility complex (MHC) molecule.

Exemplary antigen receptors, including CARs and recombinant TCRs, as well as methods for engineering and introducing the receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., 2013; Davila et al., 2013; Turtle et al., 2012; Wu et al., 2012. In some aspects, the genetically engineered antigen receptors include a CAR as described in U.S. Pat. No. 7,446,190, and those described in International Patent Application Publication No.: WO/2014055668 A1.

1. Chimeric Antigen Receptors

In some embodiments, the CAR comprises: a) an intracellular signaling domain, b) a transmembrane domain, and c) an extracellular domain comprising an antigen binding region.

In some embodiments, the engineered antigen receptors include CARs, including activating or stimulatory CARs, costimulatory CARs (see WO2014/055668), and/or inhibitory CARs (iCARs, see Fedorov et al., 2013). The CARs generally include an extracellular antigen (or ligand) binding domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone.

Certain embodiments of the present disclosure concern the use of nucleic acids, including nucleic acids encoding an antigen-specific CAR polypeptide, including a CAR that has been humanized to reduce immunogenicity (hCAR), comprising an intracellular signaling domain, a transmembrane domain, and an extracellular domain comprising one or more signaling motifs. In certain embodiments, the CAR may recognize an epitope comprising the shared space between one or more antigens. In certain embodiments, the binding region can comprise complementary determining regions of a monoclonal antibody, variable regions of a monoclonal antibody, and/or antigen binding fragments thereof. In another embodiment, that specificity is derived from a peptide (e.g., cytokine) that binds to a receptor.

It is contemplated that the human CAR nucleic acids may be human genes used to enhance cellular immunotherapy for human patients. In a specific embodiment, the disclosure includes a full-length CAR cDNA or coding region. The antigen binding regions or domain can comprise a fragment of the V_{H} and V_{L} chains of a single-chain variable fragment (scFv) derived from a particular human monoclonal antibody, such as those described in U.S. Pat. No. 7,109,304, incorporated herein by reference. The fragment can also be any number of different antigen binding domains of a human antigen-specific antibody. In a more specific embodiment, the fragment is an antigen-specific scFv encoded by a sequence that is optimized for human codon usage for expression in human cells.

The arrangement could be multimeric, such as a diabody or multimers. The multimers are most likely formed by cross pairing of the variable portion of the light and heavy chains into a diabody. The hinge portion of the construct can have multiple alternatives from being totally deleted, to having the first cysteine maintained, to a proline rather than a serine substitution, to being truncated up to the first cysteine. The Fc portion can be deleted. Any protein that is stable and/or dimerizes can serve this purpose. One could use just one of the Fc domains, e.g., either the CH2 or CH3 domain from human immunoglobulin. One could also use the hinge, CH2 and CH3 region of a human immunoglobulin that has been modified to improve dimerization. One could also use just the hinge portion of an immunoglobulin. One could also use portions of CD8alpha.

In some embodiments, the CAR nucleic acid comprises a sequence encoding other costimulatory receptors, such as a transmembrane domain and a modified CD28 intracellular signaling domain. Other costimulatory receptors include, but are not limited to one or more of CD28, CD27, OX-40 (CD134), and 4-1BB (CD137).

In some embodiments, CAR is constructed with a specificity for a particular antigen (or marker or ligand), such as an antigen expressed in a particular cell type to be targeted by adoptive therapy, e.g., a cancer marker, and/or an antigen intended to induce a dampening response, such as an antigen expressed on a normal or non-diseased cell type. Thus, the CAR typically includes in its extracellular portion one or more antigen binding molecules, such as one or more antigen-binding fragment, domain, or portion, or one or more antibody variable domains, and/or antibody molecules. In some embodiments, the CAR includes an antigen-binding portion or portions of an antibody molecule, such as a single-chain antibody fragment (scFv) derived from the variable heavy (VH) and variable light (VL) chains of a monoclonal antibody (mAb).

The sequence of the open reading frame encoding the chimeric receptor can be obtained from a genomic DNA source, a cDNA source, or can be synthesized (e.g., via PCR), or combinations thereof. Depending upon the size of the genomic DNA and the number of introns, it may be desirable to use cDNA or a combination thereof as it is found that introns stabilize the mRNA. Also, it may be further advantageous to use endogenous or exogenous non-coding regions to stabilize the mRNA.

It is contemplated that the chimeric construct can be introduced into immune cells as naked DNA or in a suitable vector. Methods of stably transfecting cells by electroporation using naked DNA are known in the art. See, e.g., U.S. Pat. No. 6,410,319. Naked DNA generally refers to the DNA encoding a chimeric receptor contained in a plasmid expression vector in proper orientation for expression.

Alternatively, a viral vector (e.g., a retroviral vector, adenoviral vector, adeno-associated viral vector, or lentiviral vector) can be used to introduce the chimeric construct into immune cells. Suitable vectors for use in accordance with the method of the present disclosure are non-replicating in the immune cells. A large number of vectors are known that are based on viruses, where the copy number of the virus maintained in the cell is low enough to maintain the viability of the cell, such as, for example, vectors based on HIV, SV40, EBV, HSV, or BPV.

In some aspects, the antigen-specific binding, or recognition component is linked to one or more transmembrane and intracellular signaling domains. In some embodiments, the CAR includes a transmembrane domain fused to the extracellular domain of the CAR. In one embodiment, the transmembrane domain that naturally is associated with one of the domains in the CAR is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 zeta, CD3 epsilon, CD3 gamma, and CD3 delta. Alternatively, the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain.

In specific embodiments, the present CAR constructs comprise a light chain-linker-heavy chain-hinge-transmembrane domain-signaling domain. The linkers may comprise, consist of, or consist essentially of Linker 1 (SEQ ID NOs: 44 or 45), Linker 2 (SEQ ID NOs:46 or 47), Linker 3 (SEQ ID NOs:48 or 49), or Linker 4 (SEQ ID NOs:50 or 51). The hinge may comprise, consist of, or consist essentially of CD8 Hinge 1 (SEQ ID NOs:52 or 53), CD8 Hinge 2 (SEQ ID NOs:54 or 55), CD8 Hinge 3 (SEQ ID NOs:56 or 57), CD28 Hinge (SEQ ID NOs:58 or 59), IgG4 Hinge (SEQ ID NOs:60 or 61), IgG4 CH2 (SEQ ID NOs:62 or 63), IgG4 CH2CH3 (SEQ ID NOs:64 or 65), or IgG4 CH1CH2CH3 (SEQ ID NOs:66 or 67). The transmembrane domain may comprise, consist of, or consist essentially of CD8 TM1 (SEQ ID NOs:68 or 69), CD8 TM2 (SEQ ID NOs:70 or 71), CD28 TM (SEQ ID NOs:72 or 73) or CD8α TM (SEQ ID NO: 87). In specific cases, a CD8α TM is utilized that lacks the amino acid sequence of LYC and/or NHRN (including in succession), such as at its C-terminus. The signaling domains may comprise, consist of, or consist essentially of CD28 (SEQ ID NOs: 74 or 75), 4-1BB (SEQ ID NOs:76 or 77), OX-40 (SEQ ID NOs:78 or 79), and/or CD3 intracellular (SEQ ID NOs:80 or 81). The CAR constructs may further comprise, consist of, or consist essentially of GFP (SEQ ID NOs:82 or 83), T2A (SEQ ID NOs:84 or 85), and/or EGFR (SEQ ID NOs:40 or 41). Exemplary heavy chain (HC), linker, and light chain (LC) combinations may include but are not limited to: LC-Linker1-HC; LC-Linker2-HC; LC-Linker3-HC; LC-Linker4-HC; HC-Linker1-LC; HC-Linker2-LC; HC-Linker3-LC; or HC-Linker4-LC.

2. T Cell Receptor (TCR)

In some embodiments, the genetically engineered antigen receptors include recombinant TCRs and/or TCRs cloned from naturally occurring T cells. A “T cell receptor” or “TCR” refers to a molecule that contains variable α and β chains (also known as TCRα and TCRβ, respectively) or variable γ and δ chains (also known as TCRγ and TCRδ, respectively) and that is capable of specifically binding to an antigen peptide bound to a MHC receptor. In some embodiments, the TCR is in the αβ form.

Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et al, 1997). For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the αβ form or γδ form.

Thus, for purposes herein, reference to a TCR includes any TCR or functional fragment, such as an antigen-binding portion of a TCR that binds to a specific antigenic peptide bound in an MHC molecule, i.e. MHC-peptide complex. An “antigen-binding portion” or antigen-binding fragment” of a TCR, which can be used interchangeably, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable a chain and variable β chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) (see, e.g., Jores et al., 1990; Chothia et al., 1988; Lefranc et al., 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the β-chain can contain a further hypervariability (HV4) region.

In some embodiments, the TCR chains contain a constant domain. For example, like immunoglobulins, the extracellular portion of TCR chains (e.g., a-chain, β-chain) can contain two immunoglobulin domains, a variable domain (e.g., Vₐ or Vp; typically amino acids 1 to 116 based on Kabat numbering Kabat et al., “Sequences of Proteins of Immunological Interest, US Dept. Health and Human Services, Public Health Service National Institutes of Health, 1991, 5^{th} ed.) at the N-terminus, and one constant domain (e.g., α-chain constant domain or Cₐ, typically amino acids 117 to 259 based on Kabat, β-chain constant domain or Cp, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the α and β chains such that the TCR contains two disulfide bonds in the constant domains.

In some embodiments, the TCR chains can contain a transmembrane domain. In some embodiments, the transmembrane domain is positively charged. In some cases, the TCR chains contains a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (γ, δ, and ε) in mammals and the ζ-chain. For example, in mammals the complex can contain a CD3γ chain, a CD3δ chain, two CD3ε chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T cell receptor chains. The intracellular tails of the CD3γ, CD3δ, and CD3ε chains each contain a single conserved motif known as an immunoreceptor tyrosine-based activation motif or ITAM, whereas each CD3ζ chain has three. Generally, ITAMs are involved in the signaling capacity of the TCR complex. These accessory molecules have negatively charged transmembrane regions and play a role in propagating the signal from the TCR into the cell. The CD3- and ζ-chains, together with the TCR, form what is known as the T cell receptor complex.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds. In some embodiments, a TCR for a target antigen (e.g., a cancer antigen) is identified and introduced into the cells. In some embodiments, nucleic acid encoding the TCR can be obtained from a variety of sources, such as by polymerase chain reaction (PCR) amplification of publicly available TCR DNA sequences. In some embodiments, the TCR is obtained from a biological source, such as from cells such as from a T cell (e.g. cytotoxic T cell), T cell hybridomas or other publicly available source. In some embodiments, the T cells can be obtained from in vivo isolated cells. In some embodiments, a high-affinity T cell clone can be isolated from a patient, and the TCR isolated. In some embodiments, the T cells can be a cultured T cell hybridoma or clone. In some embodiments, the TCR clone for a target antigen has been generated in transgenic mice engineered with human immune system genes (e.g., the human leukocyte antigen system, or HLA). In some embodiments, phage display is used to isolate TCRs against a target antigen. In some embodiments, the TCR or antigen-binding portion thereof can be synthetically generated from knowledge of the sequence of the TCR.

C. Methods of Delivery

One of skill in the art would be well-equipped to construct a vector through standard recombinant techniques (see, for example, Sambrook et al., 2001 and Ausubel et al., 1996, both incorporated herein by reference) for the expression of any antigen receptors of the present disclosure. Vectors include but are not limited to, plasmids, cosmids, viruses (bacteriophage, animal viruses, and plant viruses), and artificial chromosomes (e.g., YACs), such as retroviral vectors (e.g. derived from Moloney murine leukemia virus vectors (MoMLV), MSCV, SFFV, MPSV, SNV etc), lentiviral vectors (e.g. derived from HIV-1, HIV-2, SIV, BIV, FIV etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumor virus vectors, Rous sarcoma virus vectors, parvovirus vectors, polio virus vectors, vesicular stomatitis virus vectors, maraba virus vectors and group B adenovirus enadenotucirev vectors.

1. Viral Vectors

Viral vectors encoding an antigen receptor may be provided in certain aspects of the present disclosure. In generating recombinant viral vectors, non-essential genes are typically replaced with a gene or coding sequence for a heterologous (or non-native) protein. A viral vector is a kind of expression construct that utilizes viral sequences to introduce nucleic acid and possibly proteins into a cell. The ability of certain viruses to infect cells or enter cells via receptor mediated-endocytosis, and to integrate into host cell genomes and express viral genes stably and efficiently have made them attractive candidates for the transfer of foreign nucleic acids into cells (e.g., mammalian cells). Non-limiting examples of virus vectors that may be used to deliver a nucleic acid of certain aspects of the present disclosure are described below.

Lentiviruses are complex retroviruses, which, in addition to the common retroviral genes gag, pol, and env, contain other genes with regulatory or structural function. Lentiviral vectors are well known in the art (see, for example, U.S. Pat. No. 6,013,516 and

Recombinant lentiviral vectors are capable of infecting non-dividing cells and can be used for both in vivo and ex vivo gene transfer and expression of nucleic acid sequences. For example, recombinant lentivirus capable of infecting a non-dividing cell—wherein a suitable host cell is transfected with two or more vectors carrying the packaging functions, namely gag, pol and env, as well as rev and tat—is described in U.S. Pat. No. 5,994,136, incorporated herein by reference.

2. Regulatory Elements

Expression cassettes included in vectors useful in the present disclosure in particular contain (in a 5′-to-3′ direction) a eukaryotic transcriptional promoter operably linked to a protein-coding sequence, splice signals including intervening sequences, and a transcriptional termination/polyadenylation sequence. The promoters and enhancers that control the transcription of protein encoding genes in eukaryotic cells are composed of multiple genetic elements. The cellular machinery is able to gather and integrate the regulatory information conveyed by each element, allowing different genes to evolve distinct, often complex patterns of transcriptional regulation. A promoter used in the context of the present disclosure includes constitutive, inducible, and tissue-specific promoters.

a. Promoter/Enhancers

The expression constructs provided herein comprise a promoter to drive expression of the antigen receptor. A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30110 bp− upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the βlactamase (penicillinase), lactose and tryptophan (trp-) promoter systems. In addition to producing nucleic acid sequences of promoters and enhancers synthetically, sequences may be produced using recombinant cloning and/or nucleic acid amplification technology, including PCR™, in connection with the compositions disclosed herein. Furthermore, it is contemplated that the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally, any promoter/enhancer combination (as per, for example, the Eukaryotic Promoter Data Base EPDB, through world wide web at epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e. g., beta actin promoter, GADPH promoter, metallothionein promoter; and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at Genbank, accession no. X05244, nucleotide 283-341) or a mouse mammary tumor promoter (available from the ATCC, Cat. No. ATCC 45007). In certain embodiments, the promoter is CMV IE, dectin-1, dectin-2, human CD11c, F4/80, SM22, RSV, SV40, Ad MLP, beta-actin, MHC class I or MHC class II promoter, however any other promoter that is useful to drive expression of the therapeutic gene is applicable to the practice of the present disclosure.

In certain aspects, methods of the disclosure also concern enhancer sequences, i.e., nucleic acid sequences that increase a promoter's activity and that have the potential to act in cis, and regardless of their orientation, even over relatively long distances (up to several kilobases away from the target promoter). However, enhancer function is not necessarily restricted to such long distances as they may also function in close proximity to a given promoter.

b. Initiation Signals and Linked Expression

A specific initiation signal also may be used in the expression constructs provided in the present disclosure for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments, the use of internal ribosome entry sites (IRES) elements are used to create multigene, or polycistronic, messages. IRES elements are able to bypass the ribosome scanning model of 5′ methylated Cap dependent translation and begin translation at internal sites. IRES elements from two members of the picornavirus family (polio and encephalomyocarditis) have been described, as well an IRES from a mammalian message. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Additionally, certain 2A sequence elements could be used to create linked- or co-expression of genes in the constructs provided in the present disclosure. For example, cleavage sequences could be used to co-express genes by linking open reading frames to form a single cistron. An exemplary cleavage sequence is the F2A (Foot-and-mouth disease virus 2A) or a “2A-like” sequence (e.g., Thosea asigna virus 2A; T2A).

3. Origins of Replication

In order to propagate a vector in a host cell, it may contain one or more origins of replication sites (often termed “ori”), for example, a nucleic acid sequence corresponding to oriP of EBV as described above or a genetically engineered oriP with a similar or elevated function in programming, which is a specific nucleic acid sequence at which replication is initiated. Alternatively a replication origin of other extra-chromosomally replicating virus as described above or an autonomously replicating sequence (ARS) can be employed.

4. Selection and Screenable Markers

In some embodiments, cells containing a construct of the present disclosure may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

5. Other Methods of Nucleic Acid Delivery

In addition to viral delivery of the nucleic acids encoding the antigen receptor, the following are additional methods of recombinant gene delivery to a given host cell and are thus considered in the present disclosure.

Introduction of a nucleic acid, such as DNA or RNA, into the immune cells of the current disclosure may use any suitable methods for nucleic acid delivery for transformation of a cell, as described herein or as would be known to one of ordinary skill in the art. Such methods include, but are not limited to, direct delivery of DNA such as by ex vivo transfection, by injection, including microinjection); by electroporation; by calcium phosphate precipitation; by using DEAE-dextran followed by polyethylene glycol; by direct sonic loading; by liposome mediated transfection and receptor-mediated transfection; by microprojectile bombardment; by agitation with silicon carbide fibers; by Agrobacterium-mediated transformation; by desiccation/inhibition-mediated DNA uptake, and any combination of such methods. Through the application of techniques such as these, organelle(s), cell(s), tissue(s) or organism(s) may be stably or transiently transformed.

### V. METHODS OF TREATMENT

Certain aspects of the present embodiments can be used to prevent or treat a disease or disorder associated with CD79b signaling, including one in which killing of CD79b-positive cells would ameliorate at least one symptom of the disease or disorder. Signaling of CD79b may be reduced by any suitable compositions to prevent cancer cell proliferation. In particular cases, such substances would be an anti-CD79b antibody or anti-CD79b CAR-expressing cells.

In some embodiments, the present disclosure provides methods for immunotherapy comprising administering an effective amount of the compositions that comprise the antibodies, including at least CAR T cells, of the present disclosure. In one embodiment, a medical disease or disorder is treated by administration of a CAR-expressing cell population that elicits an immune response. In certain embodiments of the present disclosure, cancer is treated by administration of a CAR immune cell population that elicits an immune response. Provided herein are methods for treating or delaying progression of cancer in an individual comprising administering to the individual an effective amount of an antigen-specific cell therapy. The present methods may be applied for the treatment of immune disorders, solid cancers, and hematologic cancers, as examples. Specifically, the cancer may be a B cell malignancy, such as B cell acute lymphoblastic leukemia (ALL), diffuse, large B cell lymphoma, follicular lymphoma, marginal zone lymphoma, lymphoplasmacytic lymphoma, Burkitt lymphoma, and chronic lymphocytic leukemia.

Tumors for which the present treatment methods are useful include any malignant cell type, such as those found in a solid tumor or a hematological tumor. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

Certain embodiments concern methods of treatment of leukemia. Leukemia is a cancer of the blood or bone marrow and is characterized by an abnormal proliferation (production by multiplication) of blood cells, usually white blood cells (leukocytes). It is part of the broad group of diseases called hematological neoplasms. Leukemia is a broad term covering a spectrum of diseases. Leukemia is clinically and pathologically split into its acute and chronic forms.

In some embodiments of the methods of the present disclosure, activated CD4 and/or CD8 T cells in the individual are characterized by γ-IFN producing CD4 and/or CD8 T cells and/or enhanced cytolytic activity relative to prior to the administration of the combination. γ-IFN may be measured by any means known in the art, including, e.g., intracellular cytokine staining (ICS) involving cell fixation, permeabilization, and staining with an antibody against γ-IFN. Cytolytic activity may be measured by any means known in the art, e.g., using a cell killing assay with mixed effector and target cells.

In some embodiments, the subject can be administered nonmyeloablative lymphodepleting chemotherapy prior to the T cell therapy. The nonmyeloablative lymphodepleting chemotherapy can be any suitable such therapy, which can be administered by any suitable route. The nonmyeloablative lymphodepleting chemotherapy can comprise, for example, the administration of cyclophosphamide and fludarabine, particularly if the cancer is melanoma, which can be metastatic. An exemplary route of administering cyclophosphamide and fludarabine is intravenously. Likewise, any suitable dose of cyclophosphamide and fludarabine can be administered. In particular aspects, around 60 mg/kg of cyclophosphamide is administered for two days after which around 25 mg/m² fludarabine is administered for five days.

In certain embodiments, a T cell growth factor that promotes the growth and activation of the autologous T cells is administered to the subject either concomitantly with the autologous T cells or subsequently to the autologous T cells. The T cell growth factor can be any suitable growth factor that promotes the growth and activation of the autologous T cells. Examples of suitable T-cell growth factors include interleukin (IL)-2, IL-7, IL-15, and/or IL-12, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2. IL-12 is a particular T-cell growth factor.

Therapeutically effective amounts of immune cells can be administered by a number of routes, including parenteral administration, for example, intravenous, intraperitoneal, intramuscular, intrasternal, or intraarticular injection, or infusion.

Intratumoral injection, or injection into the tumor vasculature is specifically contemplated for discrete, solid, accessible tumors. Local, regional or systemic administration also may be appropriate. For tumors of >4 cm, the volume to be administered will be about 4-10 ml (in particular 10 ml), while for tumors of <4 cm, a volume of about 1-3 ml will be used (in particular 3 ml). Multiple injections delivered as single dose comprise about 0.1 to about 0.5 ml volumes.

The T cell population can be administered in treatment regimens consistent with the disease, for example a single or a few doses over one to several days to ameliorate a disease state or periodic doses over an extended time to inhibit disease progression and prevent disease recurrence. The precise dose to be employed in the formulation will also depend on the route of administration, and the seriousness of the disease or disorder, and should be decided according to the judgment of the practitioner and each patient's circumstances. The therapeutically effective amount of T cells will be dependent on the subject being treated, the severity and type of the affliction, and the manner of administration. In some embodiments, doses that could be used in the treatment of human subjects range from at least 3.8×10⁴, at least 3.8×10⁵, at least 3.8×10⁶, at least 3.8×10⁷, at least 3.8×10⁸, at least 3.8×10⁹, or at least 3.8×10¹⁰ T cells/m². In a certain embodiment, the dose used in the treatment of human subjects ranges from about 3.8×10⁹ to about 3.8×10¹⁰ T cells/m². In additional embodiments, a therapeutically effective amount of T cells can vary from about 5×10⁶ cells per kg body weight to about 7.5×10⁸ cells per kg body weight, such as about 2×10⁷ cells to about 5×10⁸ cells per kg body weight, or about 5×10⁷ cells to about 2×10⁸ cells per kg body weight. The exact amount of T cells is readily determined by one of skill in the art based on the age, weight, sex, and physiological condition of the subject. Effective doses can be extrapolated from dose-response curves derived from in vitro or animal model test systems.

In certain embodiments of the present disclosure, an effective amount of CD79b CAR-expressing immune cells are delivered to an individual in need thereof, such as an individual that has cancer. The cells then enhance the individual's immune system to attack the cancer cells. In some cases, the individual is provided with one or more doses of the immune cells. In cases where the individual is provided with two or more doses of the immune cells, the duration between the administrations should be sufficient to allow time for propagation in the individual, and in specific embodiments the duration between doses is 1, 2, 3, 4, 5, 6, 7, or more days.

In specific embodiments, the cells that have been engineered to express a CD79b CAR are provided to an individual in a therapeutically effective amount (in a range from 10³ to 10¹⁰) that ameliorates at least one symptom related to cancer cells in the individual. A therapeutically effective amount may be from 10³ to 10¹⁰, 10³ to 10⁹, 10³ to 10⁸, 10³ to 10⁷, 10³ to 10⁶, 10³ to 10⁵, 10³ to 10⁴, 10⁴ to 10¹⁰, 10⁴ to 10⁹, 10⁴ to 10⁸, 10⁴ to 10⁷, 10⁴ to 10⁶, 10⁴ to 10⁵, 10⁵ to 10¹⁰, 10⁵ to 10⁹, 10⁵ to 10⁸, 10⁵ to 10⁷, 10⁵ to 10⁶, 10⁶ to 10¹⁰, 10⁶ to 10⁹, 10⁶ to 10⁸, 10⁶ to 10⁷, 10⁷ to 10¹⁰, 10⁷ to 10⁹, 10⁷ to 10⁸, 10⁸ to 10¹⁰, 10⁸ to 10⁹, or 10⁹ to 10¹⁰ cells. Thus, in particular embodiments an individual having a certain cancer is provided once or multiple times a therapeutically effective amount of cells expressing CD79b CARs.

A. Pharmaceutical Compositions

Also provided herein are pharmaceutical compositions and formulations comprising CAR-expressing cells and a pharmaceutically acceptable carrier.

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). In one aspect, a sHASEGP is combined with one or more additional glycosaminoglycanases such as chondroitinases.

B. Combination Therapies

In certain embodiments, the compositions and methods of the present embodiments involve a T cell population in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

An immune cell therapy may be administered before, during, after, or in various combinations relative to an additional cancer therapy, such as immune checkpoint therapy. The administrations may be in intervals ranging from concurrently to minutes to days to weeks. In embodiments where the immune cell therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below an immune cell therapy is “A” and an anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present embodiments. Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gamma1I and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabine, navelbine, farnesyl-protein transferase inhibitors, transplatinum, and pharmaceutically acceptable salts, acids, or derivatives of any of the above,

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation, and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

The skilled artisan will understand that immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates (ADCs) comprise monoclonal antibodies (MAbs) that are covalently linked to cell-killing drugs and may be used in combination therapies. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. Exemplary ADC drugs include ADCETRIS® (brentuximab vedotin) and KADCYLA® (trastuzumab emtansine or T-DM1).

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, erb b2 and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies include immune adjuvants, e.g., Mycobacterium bovis, Plasmodium falciparum, dinitrochlorobenzene, and aromatic compounds); cytokine therapy, e.g., interferons α, β, and γ, IL-1, GM-CSF, and TNF; gene therapy, e.g., TNF, IL-1, IL-2, and p53; and monoclonal antibodies, e.g., anti-CD20, anti-ganglioside GM2, and anti-p185. It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies. Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody that may be used. Pembrolizumab, also known as MK-3475, Merck 3475, lambrolizumab, KEYTRUDA®, and SCH-900475, is an exemplary anti-PD-1 antibody. CT-011, also known as hBAT or hBAT-1, is also an anti-PD-1 antibody. AMP-224, also known as B7-DCIg, is a PDL2-Fc fusion soluble receptor.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. An exemplary anti-CTLA-4 antibody is ipilimumab (also known as 10D1, MDX-010, MDX-101, and Yervoy®) or antigen binding fragments and variants thereof. In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin.

### VI. ARTICLES OF MANUFACTURE OR KITS

An article of manufacture or a kit is provided comprising immune cells, antibodies, reagents, buffers, or a combination thereof is also provided herein. The article of manufacture or kit can further comprise a package insert comprising instructions for using the immune cells to treat or delay progression of cancer in an individual or to enhance immune function of an individual having cancer. Any of the antigen-specific immune cells described herein may be included in the article of manufacture or kits. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

### VII. SEQUENCES USED IN CERTAIN EMBODIMENTS

Clone T26 VH region:
Nucleotide sequence
GAGGTGCAGCTGCAGGAGTCTGGGGCTGAGCTGGTGAAGCCTGGGGCTTCAGTGAA
GATGTCCTGCAAGGCTTCTGGCTACACCTTCACCAGCTACTGGATGCACTGGGTGAA
GCAGAGGCCTGGACCAGGCCTTGAGTGGATCGGAGCAATTGATCCTTCAGATAGTT
ATACTGGCTACAATCAAAAGTTCAAGGGCAAGGCCACATTGACTGTAGACACATCC
TCCAGCACAGCCTACATGCACCTCAGCAGCCTGACATCTGAGGACTCTGCGGTCTAT
TTCTGTACAAGAAGCTACTATGGTAACTCCTGGTTTGCTTACTGGGGCCAAGGGACT
CTGGTCACTGTCTCTGCA (357 nt) (SEQ ID NO: 8)
Amino acid sequence
EVQLQESGAELVKPGASVKMSCKASGYTFTSYWMHWVKQRPGPGLEWIGAIDPSDSYT
GYNQKFKGKATLTVDTSSSTAYMHLSSLTSEDSAVYFCTRSYYGNSWFAYWGQGTLVT
VSA (SEQ ID NO: 7)
(119 aa)
T26 VH CDR1: GYTFTSYW (SEQ ID NO: 1)
T26 VH CDR2: IDPSDSYT (SEQ ID NO: 2)
T26 VH CDR3: NSWFAYWGQGTLV (SEQ ID NO: 3)
Clone T26 VL region:
Nucleotide sequence
ACATTGTGCTGACCCAATCTCCAGCTTCTTTGGCTGTGTCTCTAGGGCAGAGGGCCA
CCATCTCCTGCAAGGCCAGCCAAAGTGTTGATTATGATGGTGATAGTTATATAAACT
GGTACCAACAGAAACCAGGACAGCCACCCAAACTCCTCATCTATGCTGCATCCAAT
CTAGAATCTGGAATCCCAGCCAGGTTTAGTGCCAGTGGGTCTGGGACAGACTTCACC
CTCAACATCCATCCTGTGGAGGAGGAGGATGTTGCAGCCTATTACTGTCAGCAAAGT
AATGAGGACCCATTCACGTTCGGCTCGGGGACAAGGTTGGAAATAAAAC (330 nt)
(SEQ ID NO: 10)
Amino acid sequence
IVLTQSPASLAVSLGQRATISCKASQSVDYDGDSYINWYQQKPGQPPKLLIYAASNLESG
IPARFSASGSGTDFTLNIHPVEEEDVAAYYCQQSNEDPFTFGSGTRLEIK (110 aa) (SEQ ID
NO: 9)
T26 VL CDR1: QSVDYDGDSY (SEQ ID NO: 4)
T26 VL CDR2: AAS (SEQ ID NO: 5)
T26 VL CDR3: QQSNEDPFT (SEQ ID NO: 6)
Clone 5B VH region:
Nucleotide sequence
GAGGTGCAGCTGCAGGAGTCTGGGGCTGAGCTGGTGAAGCCTGGGGCTTCAGTGAA
GATGTCCTGCAAGGCTTCTGGCTACACCTTCACCAGCTACTGGATGCACTGGGTGAA
GCAGAGGCCTGGACAAGGCCTTGAGTGGATCGGAGCAATTGATCCTTCAGATAGTT
ATACTGGCTACAATCAAAAGTTCAAGGGCAAGGCCACATTGACTGTAGACACATCC
TCCAGCACAGCCTACATGCACCTCAGCAGCCTGACATCTGAGGACTCTGCGGTCTAT
TTCTGTACAAGAAGCTACTATGGTAACTCCTGGTTTGATTACTGGGGCCAAGGGACT
CTGGTCACTGTCTCTGCA (357 nt) (SEQ ID NO: 18)
Amino acid sequence
EVQLQESGAELVKPGASVKMSCKASGYTFTSYWMHWVKQRPGQGLEWIGAIDPSDSY
TGYNQKFKGKATLTVDTSSSTAYMHLSSLTSEDSAVYFCTRSYYGNSWFDYWGQGTLV
TVSA (119 aa) (SEQ ID NO: 17)
5B VH CDR1: GYTFTSYW (SEQ ID NO: 11)
5B VH CDR2: IDPSDSYT (SEQ ID NO: 12)
5B VH CDR3: NSWFDYWGQGTLV (SEQ ID NO: 13)
Clone 5B VL region:
Nucleotide sequence
GACATTGTGCTGACCCAATCTCCAGCTTCTTTGGCTGTGTCTCTAGGGCAGAGGGCC
ACCATCTCCTGCAAGGCCAGCCAAAGTGTTGATTATGAAGGTGATAGTTATATGAAC
TGGTACCAACAGAAACCAGGACAGCCACCCAAACTCCTCATCTATGCTGCATCCAAT
CTAGAATCTGGAATCCCAGCCAGGTTTAGTGGCAGTGGGTCTGGGACAGACTTCACC
CTCAACATCCATCCTGTGGAGGAGGAGGATGCTGCAACCTATCACTGTCAGCAAAGT
AATGAGGACCCGTTCACGTTCGGAGGGGGGACCAAGTTGGAAATAAAA (333 nt)
(SEQ ID NO: 20)
Amino acid sequence
DIVLTQSPASLAVSLGQRATISCKASQSVDYEGDSYMNWYQQKPGQPPKLLIYAASNLE
SGIPARFSGSGSGTDFTLNIHPVEEEDAATYHCQQSNEDPFTFGGGTKLEIK (111 aa) (SEQ
ID NO: 19)
5B VL CDR1: QSVDYEGDSY (SEQ ID NO: 14)
5B VL CDR2: AAS (SEQ ID NO: 15)
5B VL CDR3: QQSNEDPFT (SEQ ID NO: 16)
Clone 28B VH region:
Nucleotide sequence
GAGGTGCAGCTGCAGGAGTCTGGGGCTGAGCTGGTGAAGCCTGGGGCTTCAGTGAA
GATGTCCTGCAAGGCTTCTGGCTACACCTTCACCAGCTACTGGATGCACTGGGTGAA
GCAGAGGCCTGGACAAGGCCTTGAGTGGATCGGAGCAATTGATCCTTCAGATAGTT
ATACTGGCTACAATCAAAAGTTCAAGGGCAAGGCCACATTGACTGTAGACACATCC
TCCAGCACAGCCTACATGCACCTCAGCAGCCTGACATCTGAGGACTCTGCGGTCTAT
TTCTGTACAAGAAGCTACTATGGTAACTCCTGGTTTGCTTACTGGGGCCAAGGGACT
CTGGTCACTGTCTCTGCA (357 nt) (SEQ ID NO: 28)
Amino acid sequence
EVQLQESGAELVKPGASVKMSCKASGYTFTSYWMHWVKQRPGQGLEWIGAIDPSDSY
TGYNQKFKGKATLTVDTSSSTAYMHLSSLTSEDSAVYFCTRSYYGNSWFAYWGQGTLV
TVSA (119 aa) (SEQ ID NO: 27)
28B VH CDR1: GYTFTSYW (SEQ ID NO: 21)
28B VH CDR2: DPSDSYT (SEQ ID NO: 22)
28B VH CDR3: SWFAYWGQGTLV (SEQ ID NO: 23)
Clone 28B VL region:
Nucleotide sequence
GACATTGTGCTGACCCAATCTCCAGCTTCTTTGGCTGTGTCTCTAGGGCAGAGGGCC
ACCATCTCCTGCAAGGCCAGCCAAAGTGTTGATTATGATGGTGATAGTTATATGAAC
TGGTACCAACAGAAACCAGGACAGCCACCCAAACTCCTCATTTATGTTGCATCCAAT
CTAGAATCTGGAATCCCAGCCAGGTTTAGTGGCAGTGGGTCTGGGACAGACTTCACC
CTCAACATCCATCCTGTGGAGGAGGAGGATGCTGCAACCTATTACTGTCAGCAAAGT
AATGAGGACCCATTCACGTTCGGCTCGGGGACAAAGTTGGAAATAAAC (333 nt)
(SEQ ID NO: 30)
Amino acid sequence
DIVLTQSPASLAVSLGQRATISCKASQSVDYDGDSYMNWYQQKPGQPPKLLIYVASNLE
SGIPARFSGSGSGTDFTLNIHPVEEEDAATYYCQQSNEDPFTFGSGTKLEIN (111 aa) (SEQ
ID NO: 29)
28B VL CDR1: QSVDYDGDSY (SEQ ID NO: 24)
28B VL CDR2: VAS (SEQ ID NO: 25)
28B VL CDR3: QQSNEDPFT (SEQ ID NO: 26)
SEQ ID NO: 40
Truncated human EGFR
CGCAAAGTGTGTAACGGAATAGGTATTGGTGAATTTAAAGACTCACTCTCCATAAAT
GCTACGAATATTAAACACTTCAAAAACTGCACCTCCATCAGTGGCGATCTCCACATC
CTGCCGGTGGCATTTAGGGGTGACTCCTTCACACATACTCCTCCTCTAGATCCACAG
GAACTGGATATTCTGAAAACCGTAAAGGAAATCACAGGGTTTTTGCTGATTCAGGCT
TGGCCTGAAAACAGGACGGACCTCCATGCCTTTGAGAACCTAGAAATCATACGCGG
CAGGACCAAGCAACATGGTCAGTTTTCTCTTGCAGTCGTCAGCCTGAACATAACATC
CTTGGGATTACGCTCCCTCAAGGAGATAAGTGATGGAGATGTGATAATTTCAGGAA
ACAAAAATTTGTGCTATGCAAATACAATAAACTGGAAAAAACTGTTTGGGACCTCC
GGTCAGAAAACCAAAATTATAAGCAACAGAGGTGAAAACAGCTGCAAGGCCACAG
GCCAGGTCTGCCATGCCTTGTGCTCCCCCGAGGGCTGCTGGGGCCCGGAGCCCAGG
GACTGCGTCTCTTGCCGGAATGTCAGCCGAGGCAGGGAATGCGTGGACAAGTGCAA
CCTTCTGGAGGGTGAGCCAAGGGAGTTTGTGGAGAACTCTGAGTGCATACAGTGCC
ACCCAGAGTGCCTGCCTCAGGCCATGAACATCACCTGCACAGGACGGGGACCAGAC
AACTGTATCCAGTGTGCCCACTACATTGACGGCCCCCACTGCGTCAAGACCTGCCCG
GCAGGAGTCATGGGAGAAAACAACACCCTGGTCTGGAAGTACGCAGACGCCGGCCA
TGTGTGCCACCTGTGCCATCCAAACTGCACCTACGGATGCACTGGGCCAGGTCTTGA
AGGCTGTCCAACGAATGGGCCTAAGATCCCGTCCATCGCCACTGGGATGGTGGGGG
CCCTCCTCTTGCTGCTGGTGGTGGCCCTGGGGATCGGCCTCTTCATGCGAAGG
SEQ ID NO: 41
EGFRIII-IV
RKVCNGIGIGEFKDSLSINATNIKHFKNCTSISGDLHILPVAFRGDSFTHTPPLDPQELDIL
KTVKEITGFLLIQAWPENRTDLHAFENLEIIRGRTKQHGQFSLAVVSLNITSLGLRSLKEIS
DGDVIISGNKNLCYANTINWKKLFGTSGQKTKIISNRGENSCKATGQVCHALCSPEGCW
GPEPRDCVSCRNVSRGRECVDKCNLLEGEPREFVENSECIQCHPECLPQAMNITCTGRGP
DNCIQCAHYIDGPHCVKTCPAGVMGENNTLVWKYADAGHVCHLCHPNCTYGCTGPGL
EGCPTNGPKIPSIATGMVGALLLLLVVALGIGLFMRR
SEQ ID NO: 42
CD8Leader
ATGGCCCTGCCTGTGACAGCCCTGCTGCTGCCTCTGGCTCTGCTGCTGCATGCCGCT
AGACCC
SEQ ID NO: 43
CD8Leader
MALPVTALLLPLALLLHAARP
SEQ ID NO: 44
Linker1
GGTGGCGGAGGTTCT
SEQ ID NO: 45
Linker1
GGGGS
SEQ ID NO: 46
Linker2
GGTGGCGGAGGTTCTGGAGGTGGAGGTTCC
SEQ ID NO: 47
Linker2
GGGGSGGGGS
SEQ ID NO: 48
Linker3
GGAGGAGGTGGTAGTGGTGGAGGAGGAAGTGGAGGAGGAGGAAGT
SEQ ID NO: 49
Linker3
GGGGSGGGGSGGGGS
SEQ ID NO: 50
Linker4
GGAGGAGGTGGTAGTGGTGGAGGAGGAAGTGGTGGCGGAGGTTCTGGAGGTGGAG
GTTCC
SEQ ID NO: 51
Linker4
GGGGSGGGGSGGGGSGGGGS
SEQ ID NO: 52
CD8 Hinge 1
ACAACTACTCCAGCACCACGACCACCAACACCTGCTCCAACTATCGCATCTCAACCA
CTTTCTCTACGTCCAGAAGCATGCCGACCAGCTGCAGGAGGTGCAGTTCATACGAGA
GGTCTAGATTTCGCATGTGAT
SEQ ID NO: 53
CD8 Hinge 1
TTTPAPRPPTPAPTIASQPLSLRPEACRPAAGGAVHTRGLDFACD
SEQ ID NO: 54
CD8 Hinge 2
AAGCCCACAACTACTCCAGCACCACGACCACCAACACCTGCTCCAACTATCGCATCT
CAACCACTTTCTCTACGTCCAGAAGCATGCCGACCAGCTGCAGGAGGTGCAGTTCAT
ACGAGAGGTCTAGATTTCGCATGTGAT
SEQ ID NO: 55
CD8 Hinge 2
KPTTTPAPRPPTPAPTIASQPLSLRPEACRPAAGGAVHTRGLDFACD
SEQ ID NO: 56
CD8 Hinge 3
TTCAGCCACTTCGTGCCGGTCTTCCTGCCAGCGAAGCCCACAACTACTCCAGCACCA
CGACCACCAACACCTGCTCCAACTATCGCATCTCAACCACTTTCTCTACGTCCAGAA
GCATGCCGACCAGCTGCAGGAGGTGCAGTTCATACGAGAGGTCTAGATTTCGCATGT
GAT
SEQ ID NO: 57
CD8 Hinge 3
FSHFVPVFLPAKPTTTPAPRPPTPAPTIASQPLSLRPEACRPAAGGAVHTRGLDFACD
SEQ ID NO: 58
CD28 Hinge
ATTGAAGTTATGTATCCTCCTCCTTACCTAGACAATGAGAAGAGCAATGGAACCATT
ATCCATGTGAAAGGG
SEQ ID NO: 59
CD28 Hinge
IEVMYPPPYLDNEKSNGTIIHVKG
SEQ ID NO: 60
IgG4 Hinge
GAGTCCAAATATGGTCCCCCATGCCCATCATGCCCA
SEQ ID NO: 61
IgG4 Hinge
ESKYGPPCPSCP
SEQ ID NO: 62
IgG4 CH2
GAGTCCAAATATGGTCCCCCATGCCCATCATGCCCAGCACCTGAGTTCCTGGGGGGA
CCATCAGTCTTCCTGTTCCCCCCAAAACCCAAGGACACTCTCATGATCTCCCGGACC
CCTGAGGTCACGTGCGTGGTGGTGGACGTGAGCCAGGAAGACCCCGAGGTCCAGTT
CAACTGGTACGTGGATGGCGTGGAGGTGCATAATGCCAAGACAAAGCCGCGGGAGG
AGCAGTTCCAAAGCACGTACCGTGTGGTCAGCGTCCTCACCGTCCTGCACCAGGACT
GGCTGAACGGCAAGGAGTACAAGTGCAAGGTCTCCAACAAAGGCCTCCCGTCCTCC
ATCGAGAAAACCATCTCCAAAGCCAAAGGG
SEQ ID NO: 63
IgG4 CH2
ESKYGPPCPSCPAPEFLGGPSVFLFPPKPKDTLMISRTPEVTCVVVDVSQEDPEVQFNWY
VDGVEVHNAKTKPREEQFQSTYRVVSVLTVLHQDWLNGKEYKCKVSNKGLPSSIEKTIS
KAKG
SEQ ID NO: 64
IgG4 CH2CH3
GAGTCCAAATATGGTCCCCCATGCCCATCATGCCCAGCACCTGAGTTCCTGGGGGGA
CCATCAGTCTTCCTGTTCCCCCCAAAACCCAAGGACACTCTCATGATCTCCCGGACC
CCTGAGGTCACGTGCGTGGTGGTGGACGTGAGCCAGGAAGACCCCGAGGTCCAGTT
CAACTGGTACGTGGATGGCGTGGAGGTGCATAATGCCAAGACAAAGCCGCGGGAGG
AGCAGTTCCAAAGCACGTACCGTGTGGTCAGCGTCCTCACCGTCCTGCACCAGGACT
GGCTGAACGGCAAGGAGTACAAGTGCAAGGTCTCCAACAAAGGCCTCCCGTCCTCC
ATCGAGAAAACCATCTCCAAAGCCAAAGGGCAGCCCCGAGAGCCACAGGTGTACAC
CCTGCCCCCATCCCAGGAGGAGATGACCAAGAACCAGGTCAGCCTGACCTGCCTGG
TCAAAGGCTTCTACCCCAGCGACATCGCCGTGGAGTGGGAGAGCAATGGGCAGCCG
GAGAACAACTACAAGACCACGCCTCCCGTGCTGGACTCCGACGGCTCCTTCTTCCTC
TACAGCAGGCTCACCGTGGACAAGAGCAGGTGGCAGGAGGGGAATGTCTTCTCATG
CTCCGTGATGCATGAGGCTCTGCACAACCACTACACACAGAAGAGCCTCTCCCTGTC
TCCGGGTAAA
SEQ ID NO: 65
IgG4 CH2CH3
ESKYGPPCPSCPAPEFLGGPSVFLFPPKPKDTLMISRTPEVTCVVVDVSQEDPEVQFNWY
VDGVEVHNAKTKPREEQFQSTYRVVSVLTVLHQDWLNGKEYKCKVSNKGLPSSIEKTIS
KAKGQPREPQVYTLPPSQEEMTKNQVSLTCLVKGFYPSDIAVEWESNGQPENNYKTTPP
VLDSDGSFFLYSRLTVDKSRWQEGNVFSCSVMHEALHNHYTQKSLSLSPGK
SEQ ID NO: 66
IgG4 CH1CH2CH3
GCTAGCACCAAGGGCCCATCGGTCTTCCCCCTGGCGCCCTGCTCCAGGAGCACCTCC
GAGAGCACAGCCGCCCTGGGCTGCCTGGTCAAGGACTACTTCCCCGAACCGGTGAC
GGTGTCGTGGAACTCAGGCGCCCTGACCAGCGGCGTGCACACCTTCCCGGCTGTCCT
ACAGTCCTCAGGACTCTACTCCCTCAGCAGCGTGGTGACCGTGCCCTCCAGCAGCTT
GGGCACGAAGACCTACACCTGCAACGTAGATCACAAGCCCAGCAACACCAAGGTGG
ACAAGAGAGTTGAGTCCAAATATGGTCCCCCATGCCCATCATGCCCAGCACCTGAGT
TCCTGGGGGGACCATCAGTCTTCCTGTTCCCCCCAAAACCCAAGGACACTCTCATGA
TCTCCCGGACCCCTGAGGTCACGTGCGTGGTGGTGGACGTGAGCCAGGAAGACCCC
GAGGTCCAGTTCAACTGGTACGTGGATGGCGTGGAGGTGCATAATGCCAAGACAAA
GCCGCGGGAGGAGCAGTTCCAAAGCACGTACCGTGTGGTCAGCGTCCTCACCGTCC
TGCACCAGGACTGGCTGAACGGCAAGGAGTACAAGTGCAAGGTCTCCAACAAAGGC
CTCCCGTCCTCCATCGAGAAAACCATCTCCAAAGCCAAAGGGCAGCCCCGAGAGCC
ACAGGTGTACACCCTGCCCCCATCCCAGGAGGAGATGACCAAGAACCAGGTCAGCC
TGACCTGCCTGGTCAAAGGCTTCTACCCCAGCGACATCGCCGTGGAGTGGGAGAGC
AATGGGCAGCCGGAGAACAACTACAAGACCACGCCTCCCGTGCTGGACTCCGACGG
CTCCTTCTTCCTCTACAGCAGGCTCACCGTGGACAAGAGCAGGTGGCAGGAGGGGA
ATGTCTTCTCATGCTCCGTGATGCATGAGGCTCTGCACAACCACTACACACAGAAGA
GCCTCTCCCTGTCTCCGGGTAAA
SEQ ID NO: 67
IgG4 CH1CH2CH3
ASTKGPSVFPLAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPSCPAPEFLGGPSV
FLFPPKPKDTLMISRTPEVTCVVVDVSQEDPEVQFNWYVDGVEVHNAKTKPREEQFQST
YRVVSVLTVLHQDWLNGKEYKCKVSNKGLPSSIEKTISKAKGQPREPQVYTLPPSQEEM
TKNQVSLTCLVKGFYPSDIAVEWESNGQPENNYKTTPPVLDSDGSFFLYSRLTVDKSRW
QEGNVFSCSVMHEALHNHYTQKSLSLSPGK
SEQ ID NO: 68
CD8 TM 1
ATCTACATCTGGGCACCATTGGCTGGGACTTGTGGTGTCCTTCTCCTATCACTGGTTA
TCACCCTTTACTGC
SEQ ID NO: 69
CD8 TM 1
IYIWAPLAGTCGVLLLSLVITLYC
SEQ ID NO: 70
CD8 TM 2
ATCTACATCTGGGCACCATTGGCTGGGACTTGTGGTGTCCTTCTCCTATCACTGGTTA
TCACCCTTTACTGCAACCACAGGAAC
SEQ ID NO: 71
CD8 TM 2
IYIWAPLAGTCGVLLLSLVITLYCNHRN
SEQ ID NO: 72
CD28 TM
AAACACCTTTGTCCAAGTCCCCTATTTCCCGGACCTTCTAAGCCCTTTTGGGTGCTGG
TGGTGGTTGGTGGAGTCCTGGCTTGCTATAGCTTGCTAGTAACAGTGGCCTTTATTAT
TTTC
SEQ ID NO: 73
CD28 TM
KHLCPSPLFPGPSKPFWVLVVVGGVLACYSLLVTVAFIIF
SEQ ID NO: 74
CD28
AGAAGTAAAAGAAGTAGGCTACTTCATAGTGATTACATGAATATGACTCCTCGACG
ACCTGGTCCCACCCGTAAGCATTATCAGCCCTATGCACCACCACGAGATTTCGCAGC
CTATCGCTCC
SEQ ID NO: 75
CD28
RSKRSRLLHSDYMNMTPRRPGPTRKHYQPYAPPRDFAAYRS
SEQ ID NO: 76
4-1BB
AAACGAGGTAGAAAAAAACTTCTTTATATATTCAAACAACCATTTATGAGACCAGTA
CAAACTACTCAAGAGGAAGATGGATGTAGTTGTCGATTTCCAGAAGAAGAAGAAGG
AGGATGTGAACTG
SEQ ID NO: 77
4-1BB
RVKFSRSADAPAYKQGQNQLYNELNLGRREEYDVLDKRRGRDPEMGGKPRRKNPQEG
LYNELQKDKMAEAYSEIGMKGERRRGKGHDGLYQGLSTATKDTYDALHMQALPPR
SEQ ID NO: 78
OX-40
AGGCGCGACCAGCGGCTGCCACCTGATGCACACAAGCCACCAGGAGGAGGCTCTTT
CCGGACCCCAATCCAGGAGGAGCAGGCAGACGCACACAGCACACTGGCCAAGATC
SEQ ID NO: 79
OX-40
RRDQRLPPDAHKPPGGGSFRTPIQEEQADAHSTLAKI
SEQ ID NO: 80
CD3ζ Intracellular
AGAGTTAAATTTAGCAGAAGTGCAGATGCTCCTGCGTATAAACAGGGTCAAAACCA
ACTATATAATGAACTAAATCTAGGACGAAGAGAAGAATATGATGTTTTAGATAAAA
GACGTGGTCGAGATCCTGAAATGGGAGGAAAACCTAGAAGAAAAAATCCTCAAGA
AGGCCTATATAATGAACTACAAAAAGATAAGATGGCAGAAGCTTATAGTGAAATTG
GAATGAAAGGAGAACGTCGTAGAGGTAAAGGTCATGATGGTCTTTATCAAGGTCTT
AGTACAGCAACAAAAGATACATATGATGCACTTCATATGCAAGCACTTCCACCTCGT
SEQ ID NO: 81
CD3ζ Intracellular
RVKFSRSADAPAYKQGQNQLYNELNLGRREEYDVLDKRRGRDPEMGGKPRRKNPQEG
LYELQKDKMAEAYSEIGMKGERRRGKGHDGLYQGLSTATKDTYDALHMQALPPR
SEQ ID NO: 82
Enhanced GFP
ATGGTGAGCAAGGGCGAGGAGCTGTTCACCGGGGTGGTGCCCATCCTGGTCGAGCT
GGACGGCGACGTAAACGGCCACAAGTTCAGCGTGTCCGGCGAGGGCGAGGGCGAT
GCCACCTACGGCAAGCTGACCCTGAAGTTCATCTGCACCACCGGCAAGCTGCCCGTG
CCCTGGCCCACCCTCGTGACCACCCTGACCTACGGCGTGCAGTGCTTCAGCCGCTAC
CCCGACCACATGAAGCAGCACGACTTCTTCAAGTCCGCCATGCCCGAAGGCTACGTC
CAGGAGCGCACCATCTTCTTCAAGGACGACGGCAACTACAAGACCCGCGCCGAGGT
GAAGTTCGAGGGCGACACCCTGGTGAACCGCATCGAGCTGAAGGGCATCGACTTCA
AGGAGGACGGCAACATCCTGGGGCACAAGCTGGAGTACAACTACAACAGCCACAA
CGTCTATATCATGGCCGACAAGCAGAAGAACGGCATCAAGGTGAACTTCAAGATCC
GCCACAACATCGAGGACGGCAGCGTGCAGCTCGCCGACCACTACCAGCAGAACACC
CCCATCGGCGACGGCCCCGTGCTGCTGCCCGACAACCACTACCTGAGCACCCAGTCC
GCCCTGAGCAAAGACCCCAACGAGAAGCGCGATCACATGGTCCTGCTGGAGTTCGT
GACCGCCGCCGGGATCACTCTCGGCATGGACGAGCTGTACAAG
SEQ ID NO: 83
Enhanced GFP
MVSKGEELFTGVVPILVELDGDVNGHKFSVSGEGEGDATYGKLTLKFICTTGKLPVPWP
TLVTTLTYGVQCFSRYPDHMKQHDFFKSAMPEGYVQERTIFFKDDGNYKTRAEVKFEG
DTLVNRIELKGIDFKEDGNILGHKLEYNYNSHNVYIMADKQKNGIKVNFKIRHNIEDGSV
QLADHYQQNTPIGDGPVLLPDNHYLSTQSALSKDPNEKRDHMVLLEFVTAAGITLGMD
ELYK
SEQ ID NO: 84
T2A
GAGGGCAGAGGCAGTCTGCTGACATGCGGTGACGTGGAAGAGAATCCCGGCCCT
SEQ ID NO: 85
T2A
EGRGSLLTCGDVEENPGP
SEQ ID NO: 86
CD8α TM (nucleotide sequence)
ATCTACATCTGGGCACCATTGGCTGGGACTTGTGGTGTCCTTCTCCTATCACTGGTTA
TCACC
SEQ ID NO: 87
CD8α TM (amino acid sequence)
IYIWAPLAGTCGVLLLSLVIT

### VIII. EXAMPLES

The following examples are included to demonstrate particular embodiments of the disclosure. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Development of CD79b Antibodies and CARs

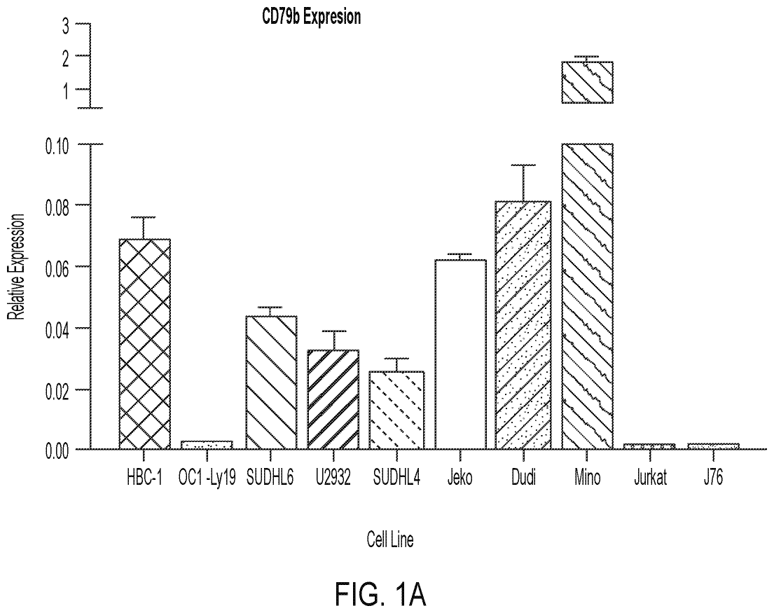

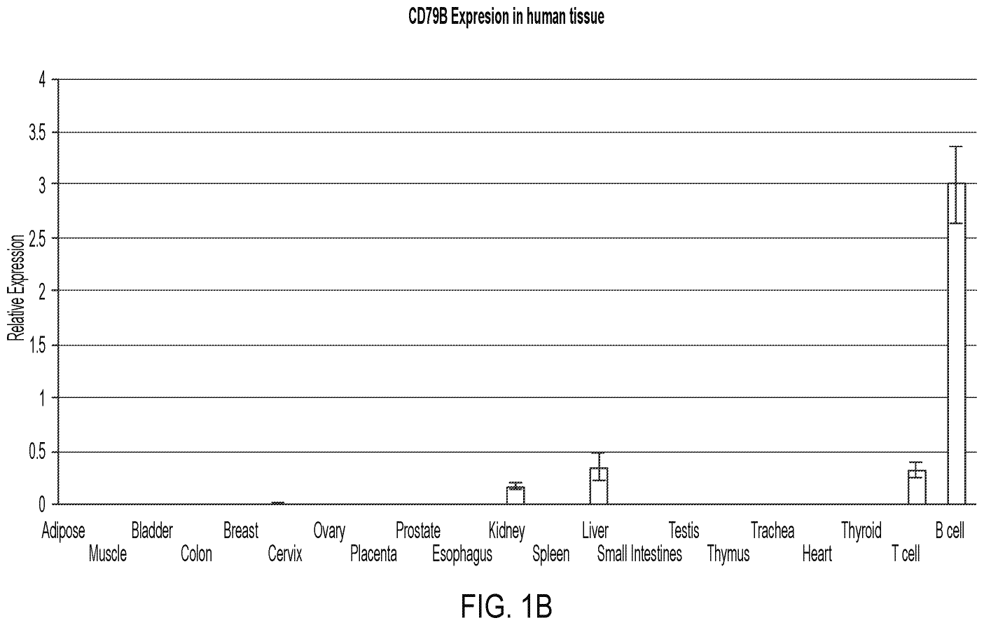

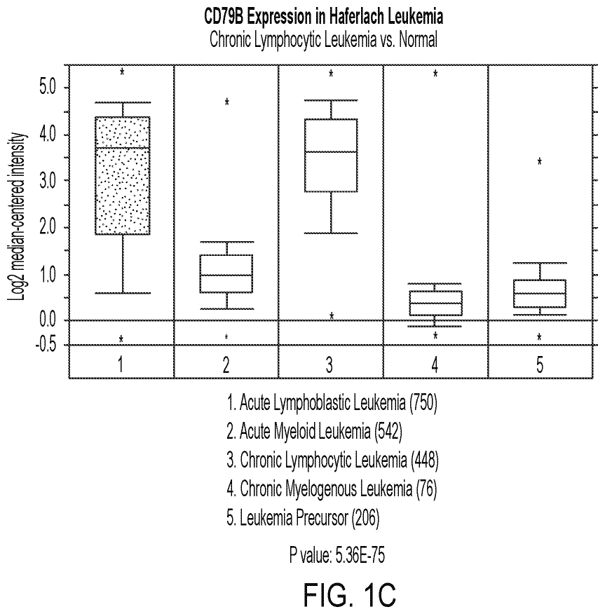

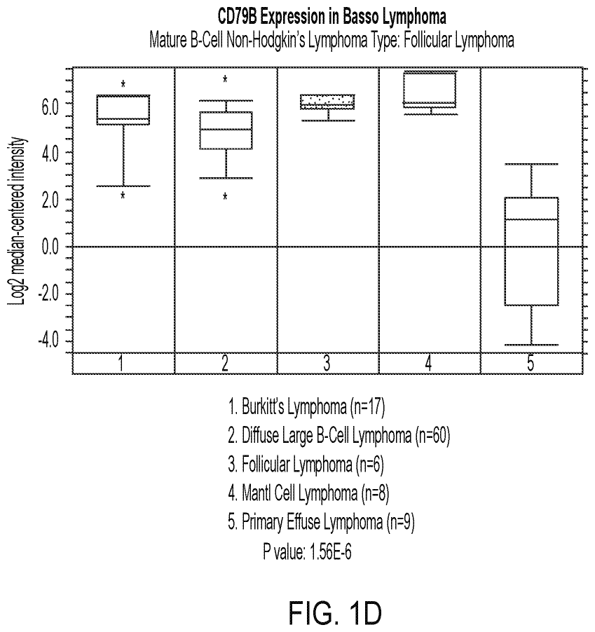

CD79b expression is restricted to B cell linage: Using real-time PCR, it was found that CD79b is expressed in a broad range of B cell lymphoma cell lines including Mino, Daudi, HBC-1, Jeko, SUDHL6, SUDHL4, and U2932 but has no expression in Jurkat and J76 T-cell lymphoma/leukemia cell lines (FIG. 1A). To determine whether CD79b was expressed on normal tissues, the FirstChoice Human Total RNA Survey Panel containing 20 normal human tissue total RNA was obtained from Applied Biosystems. Total RNA was extracted from purified B and T cells from human tonsil samples and used as positive and negative controls, respectively. It was found that CD79b transcripts are only present in lymphoid tissues such as spleen and lymph node but are absent in all nonlymphoid normal tissues (FIG. 1B).

Using publicly available gene expression datasets (Oncomine), it was found that CD79b is highly expressed in ALL and chronic lymphocytic leukemia (FIG. 1C) and multiple B cell lymphoma subtypes such as Burkitt's lymphoma, diffuse large B cell lymphoma, follicular lymphoma, and mantle cell lymphoma (FIG. 1D). The number of samples for each tumor type is shown in brackets.

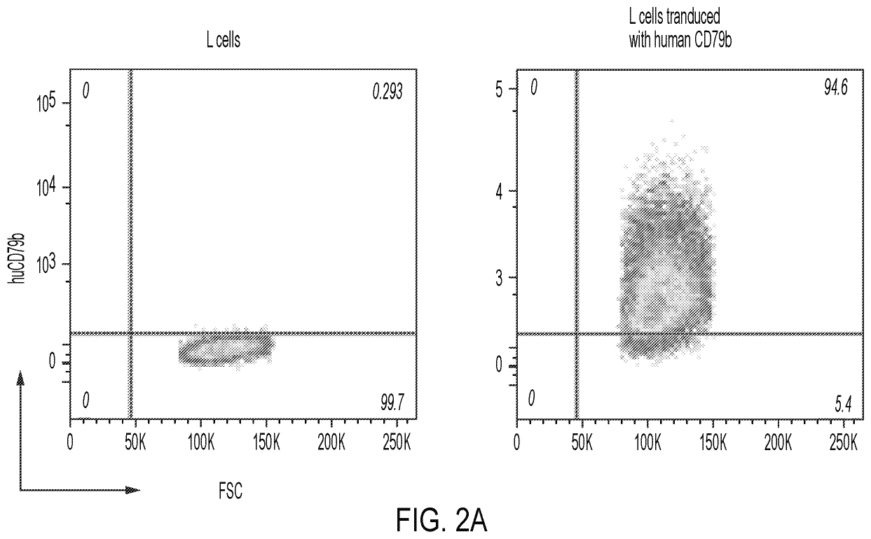

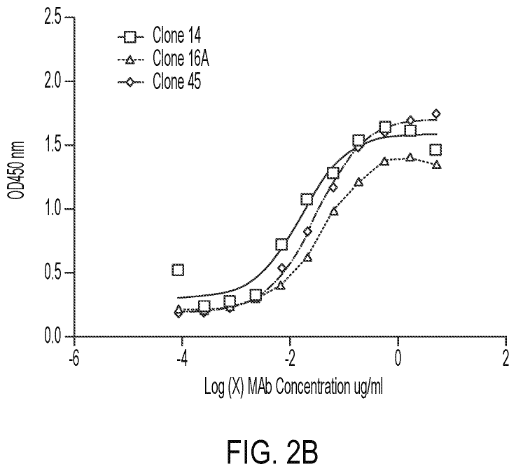

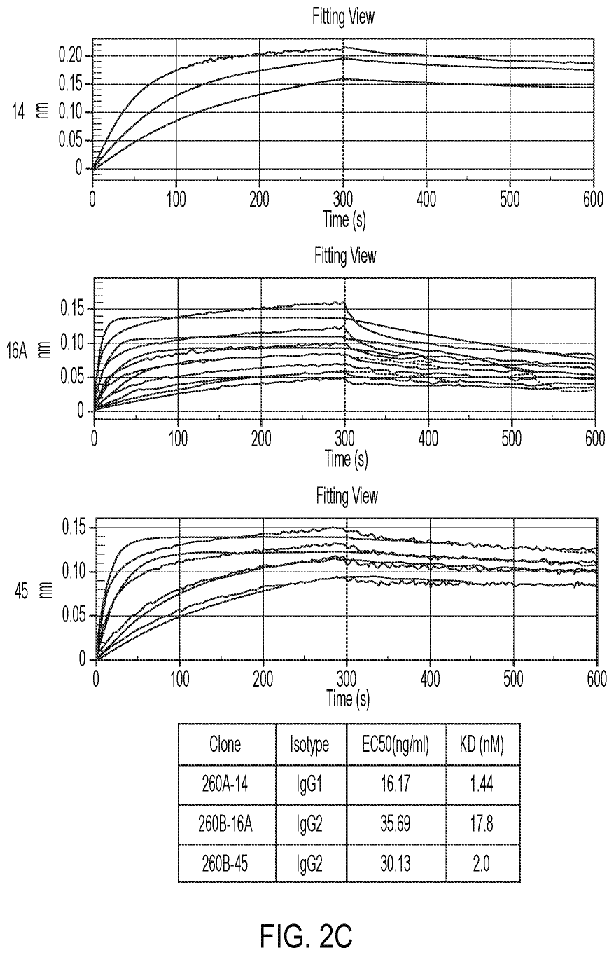

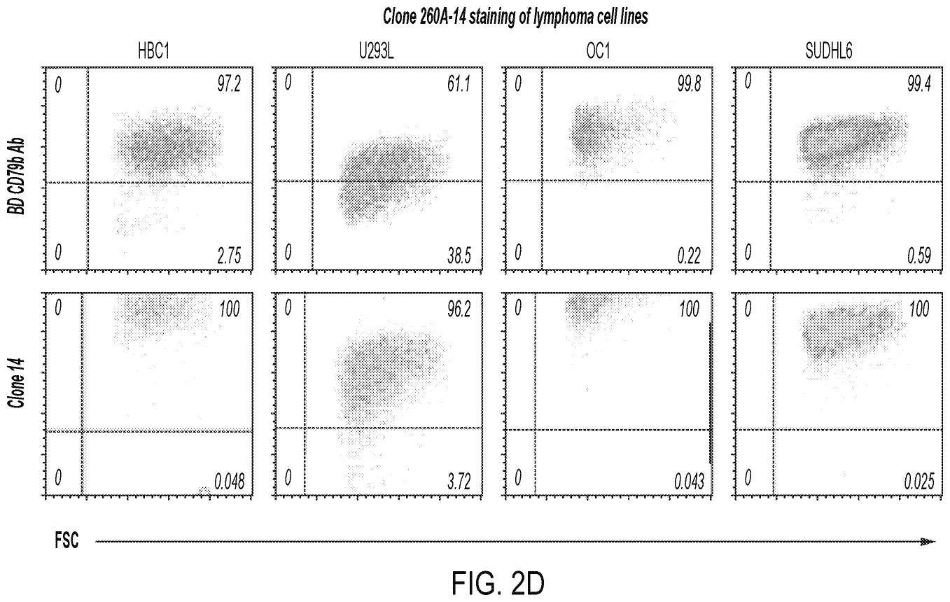

Generation of multiple monoclonal antibodies against human CD79b and identification of heavy and light chain sequences of antibodies: Anti-human CD79b monoclonal antibodies were generated by hybridoma technology by immunizing mice with human CD79b-expressing mouse fibroblast L cells (FIG. 2A). By ELISA, three clones were identified with high binding capacity to recombinant human CD79b protein (FIG. 2B). The affinity of these three monoclonal antibodies were further determined by Octet Assay and three clones, 14 (IgG1), 16A (IgG2) and 45 (IgG2) with Kd value at 1.44, 17.8 and 2.0 nM, respectively were selected for further development (FIG. 2C). The monoclonal antibody clone #14 was conjugated with a fluorochrome and shown to stain B cell lymphoma cell lines comparable to the commercial anti-CD79b antibody from BD Biosciences (FIG. 2D). Total RNA of the hybridomas of the monoclonal antibodies was extracted, cDNA was synthesized. 5′-RACE PCR (rapid amplification of cDNA ends) was used to clone V-genes for heavy and light chains. Protein sequences were predicted by DNA sequences. Hybridoma culture supernatants were purified and heavy chain and light chain protein sequences were confirmed by mass spectrometry from MD Anderson Proteomics Core Facility.

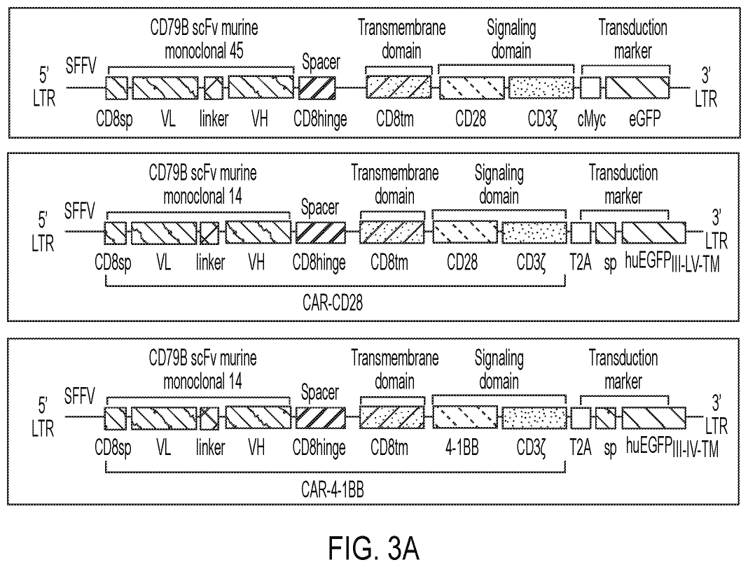

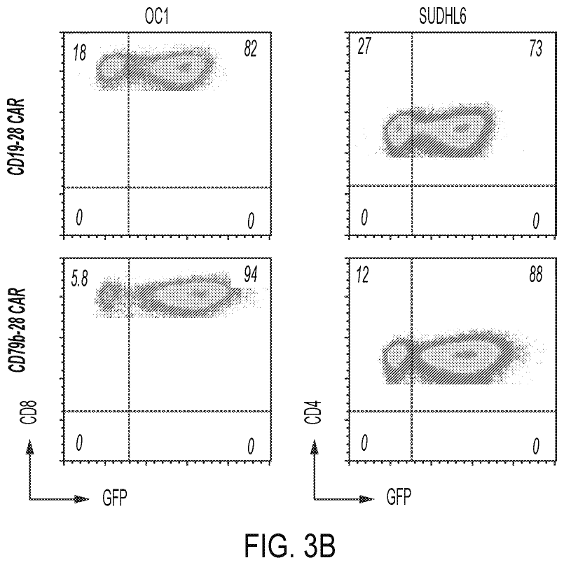

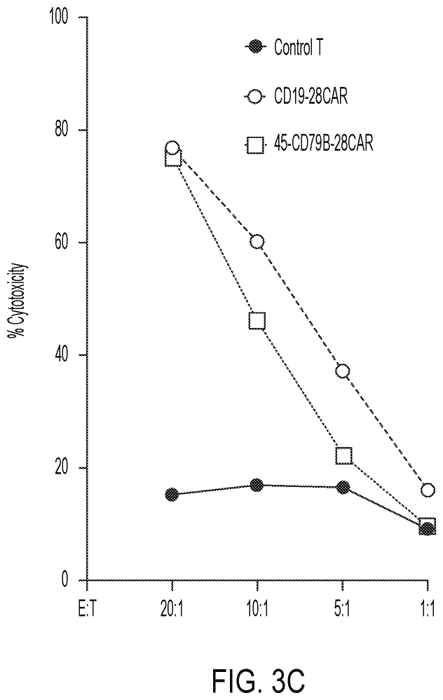

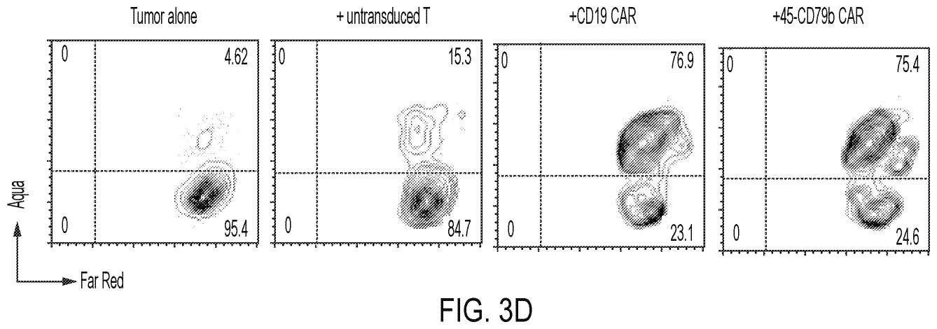

Generation of anti-CD79b CAR T cells: Using the specific sequences of single chain fragments of variable region (scFv), several constructs of anti-CD79b CAR were generated. In order to detect CAR expression in transduced T cells, CAR-enhanced green fluorescent protein (eGFP) fusion construct or the truncated human epidermal growth factor receptor (huEGFRt) were used (FIG. 3A). The latter can also serve as a safety switch to eliminate CAR T cells in case of severe toxicity. CD3-zeta (CD3z) chain was incorporated to provide signal 1 for activation of T cells and the costimulatory domains CD28 or 4-1BB were incorporated to provide signal 2 (FIG. 3A).

These constructs were cloned into lentivirus vector pHR_SFFV, which was then used to transduce primary healthy donor T cells. Representative transduction efficiency (>70%) as determined by eGFP expression in CD4⁺ and CD8⁺ T cells using Clone 45-CD79b-CD28-CAR is shown in FIG. 3B. Anti-CD19-CAR T cells were used as control.

Cytotoxic activity of CAR T cells against Daudi cells labeled with CellTrace Far Red was determined by Aqua staining in a 16-hour flow cytometry assay at the indicated Effector:Target (E:T) ratios (FIG. 3C). Representative dot plots with percent dead cells (upper right quadrant) for the various culture conditions at an E:T ratio of 20:1 is shown in FIG. 3D. The data show that both anti-CD19-CAR T cells and anti-CD79b-CAR T cells were highly cytotoxic to Daudi Burkitt lymphoma cells compared with untransduced control T cells.

Anti-CD79b CAR T cells are cytotoxic to both CD19⁺ and CD19⁻ lymphoma cells: To determine the efficacy of anti-CD79b CAR T cells against CD19 negative (CD19⁻) lymphoma cells, degranulation and cytotoxicity assays were performed with a diffuse large B cell lymphoma cell line, SUDHL6, lacking CD19. First, CD19 was knocked out by CRISPR-Cas9 (CD19KOSUDHL6) and then these lymphoma cells were transduced with CD19 splice variant lacking exon 2 (CD19Dex2) which is the binding site for anti-CD19 antibody clone, FMC63, used in anti CD19 CAR constructs.

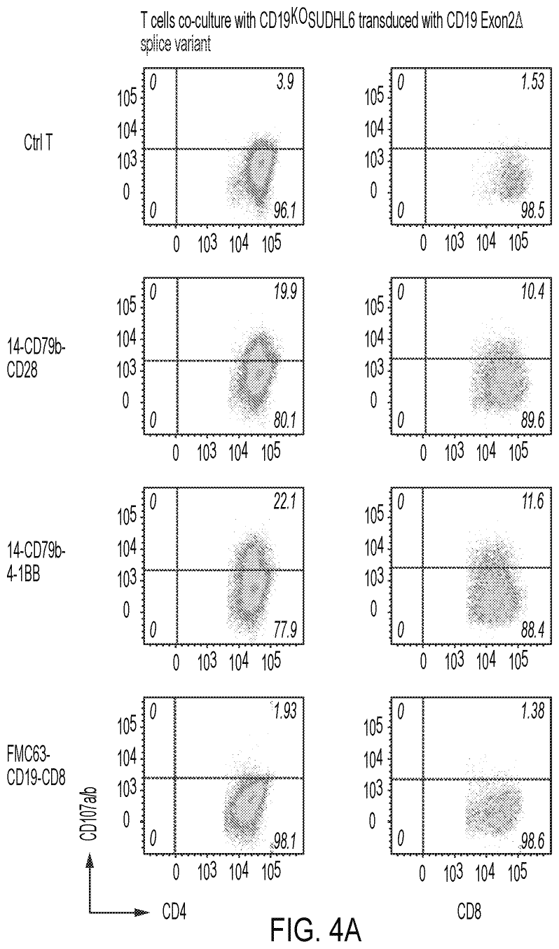

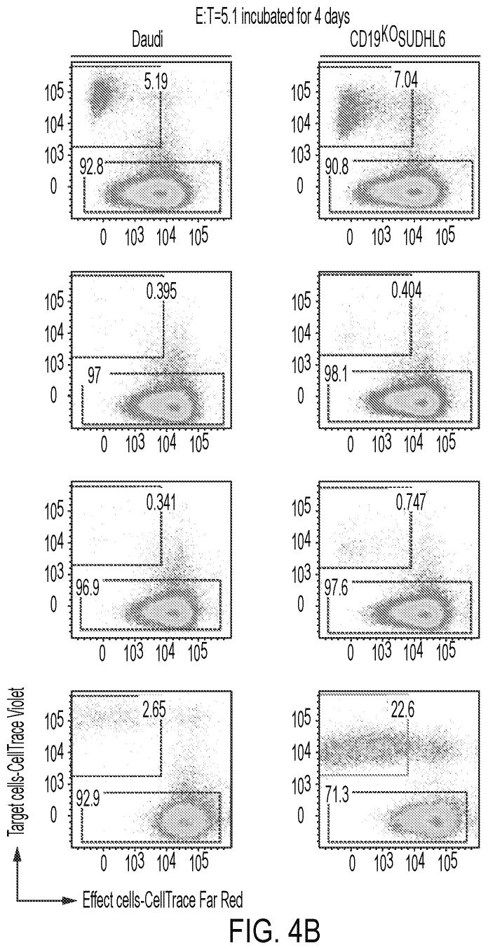

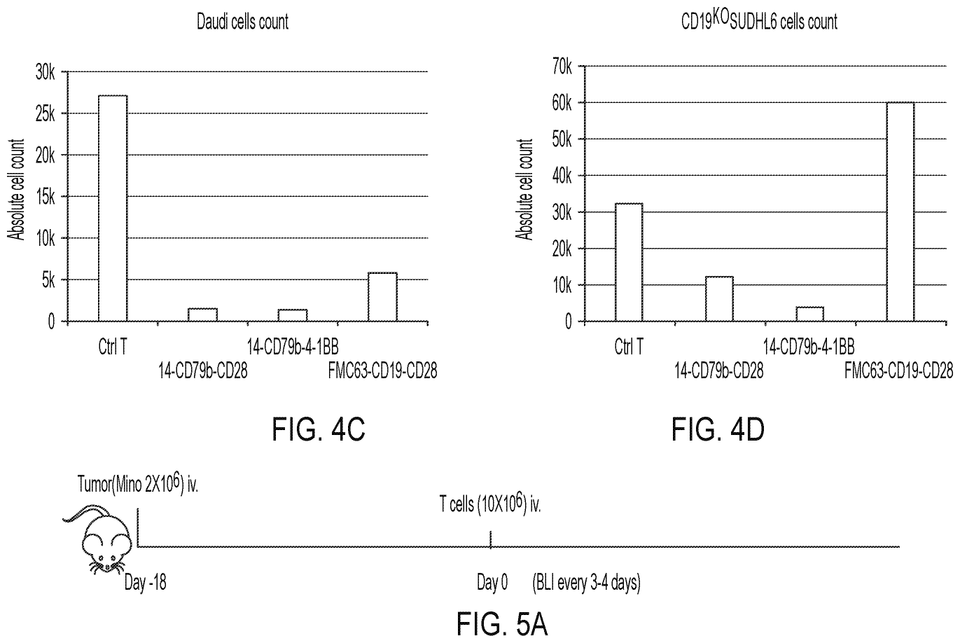

Untransduced primary T cells (Ctrl T), clone-14-CD79b-CD28 CAR, 14-CD79b-4-1BB CAR, and FMC63-CD19-CD28 CAR were co-cultured with Daudi or the above SUDHL6 cells (CD19KOSUDHL6-CD19Dex2) at an E:T ratio of 5:1. T cells were labeled with CellTrace Far Red and target cells were labeled with CellTrace Violet. After 2 hours, Golgi inhibitor and degranulation marker (CD107a/b) were added to the cultures to determine degranulation of T cells. Cytotoxic activity against tumor cells was determined after 4 days of coculture. The 14-CD79b-CD28 and 14-CD79b-4-1BB CAR T cells but not control T cells showed markedly increased degranulation and cytotoxic activity against both cell lines (FIGS. 4A and B). In contrast, FMC63-CD19-CD28 CAR was cytotoxic to CD19⁺ Daudi but not CD19KOSUDHL6-CD19Dex2 tumor cells.

Using CountBright Absolute Counting Beads for flow cytometry the absolute number of live tumor cells was also determined after the 4-day co-culture (FIG. 4C). The results were consistent with the observed percentages of live tumor cells (FIG. 4B). Representative dot plots of target cells and effector T cells are shown (FIGS. 4A and B). The experiments were repeated at least 3 times with similar results.

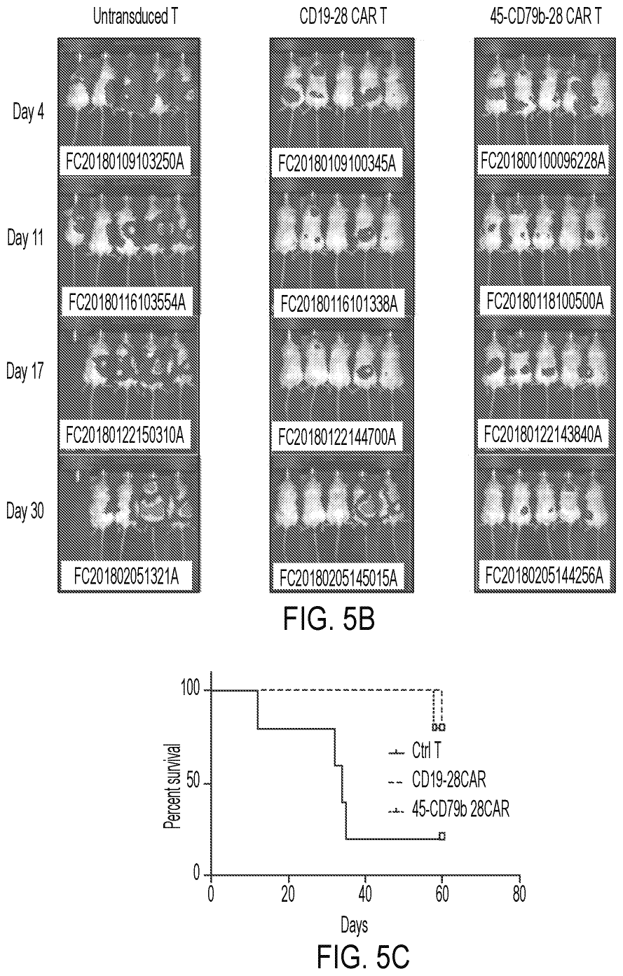

Anti-CD79b CAR T cells exhibit in vivo efficacy against lymphoma xenografts: To test the efficacy of anti-CD79b CAR T cells in vivo, Mino mantle cell lymphoma cell line expressing firefly luciferase gene was injected IV into NSG mice at 2×10⁶ tumor cells/mouse. After 18 days, mice were treated with untransduced primary T cells, anti-CD19-CD28 CAR T cells, or Clone 45 anti-CD79b-CD28 CAR T cells via tail vein injection at 10×10⁶ CARP T cells or untransduced T cells/mouse. Bioluminescence imaging was used to assess tumor burden (FIG. 5A). The results showed progressive tumor growth in mice treated with untransduced T cells. In contrast, good tumor control and markedly improved survival (p<0.05) was observed in mice treated with both anti-CD19- and anti-CD79b CAR T cells (FIGS. 5B and 5C). The in vitro results have been verified at least three times for each individual experiment and in vivo results were verified twice. Thus, anti-CD79b CAR therapy can be used for the treatment of B cell malignancy with or without CD19 expression.

### Example 2—Development of Additional CD79b Antibodies and CARs

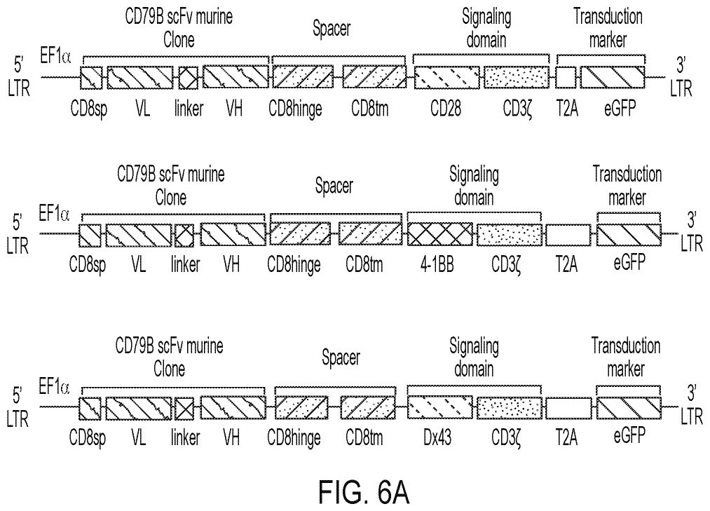

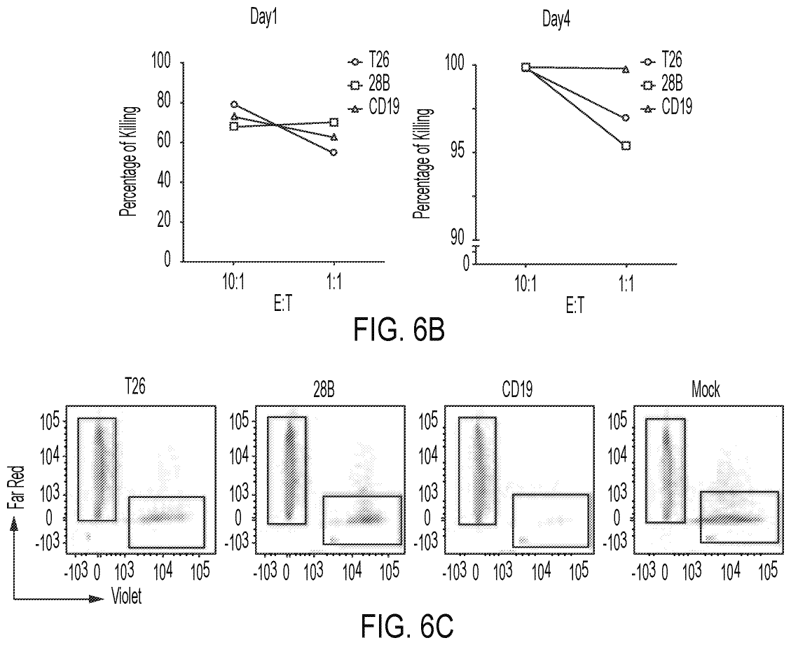

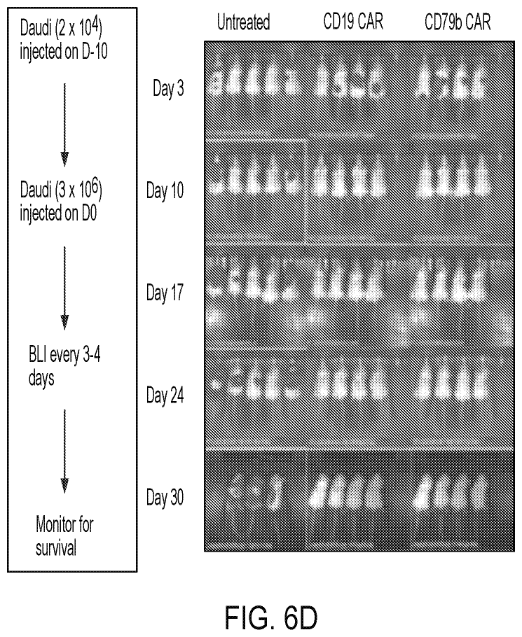

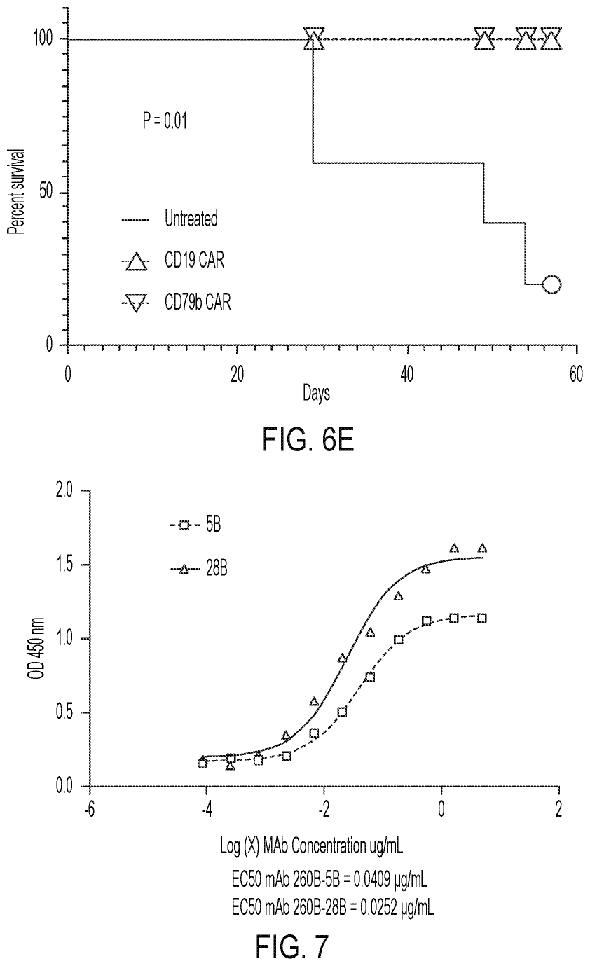

Generation of anti-CD79b CAR T cells using new anti-CD79b antibody clones: Using the specific sequences of single chain fragments of variable region (scFv) from additional antibody clones (T26, 5B, and 28B), several constructs of anti-CD79b CAR were generated. In order to detect CAR expression in transduced T cells, CAR-enhanced green fluorescent protein (eGFP) fusion construct or the truncated human epidermal growth factor receptor (huEGFRt) were used (FIG. 6A). The latter can also serve as a safety switch to eliminate CAR T cells in case of severe toxicity. CD3-zeta (CD3z) chain was incorporated to provide signal 1 for activation of T cells and the costimulatory domains CD28 or 4-1BB or OX-40 were incorporated to provide signal 2 (FIG. 6A).

Anti-CD79b CAR T cells exhibit cytotoxicity in vitro against Daudi lymphoma cells. The above CAR constructs (T26 and 28B) were cloned into lentivirus vector pLVEG, which was then used to transduce primary healthy donor T cells to generate anti-CD79b-CAR T cells. Anti-CD19-CAR T cells were used as control. Cytotoxic activity of CAR T cells (labeled with CellTrace Far Red) against Daudi cells (labeled with CellTrace Violet) was determined by a flow cytometry assay at the indicated Effector:Target (E:T) ratios after co-culture for 1 day and 4 days (FIG. 6B). Representative dot plots are shown in FIG. 6C. The data show that both anti-CD19-CART cells and anti-CD79b-CAR T cells were highly cytotoxic to Daudi Burkitt lymphoma cells.

Anti-CD79b CAR T cells exhibit in vivo efficacy against Daudi lymphoma xenografts: To test the efficacy of anti-CD79b CAR T cells (clone T26) in vivo, Daudi Burkitt lymphoma cell line expressing firefly luciferase gene was injected IV into NSG mice at 2×10⁴ tumor cells/mouse. After 10 days, mice were either untreated or treated with, anti-CD19-CAR T cells, or Clone T26 anti-CD79b-CAR T cells via tail vein injection at 3×10⁶ CAR+ T cells/mouse. Bioluminescence imaging was used to assess tumor burden (FIG. 6D). The results showed progressive tumor growth in untreated mice. In contrast, good tumor control and markedly improved survival (p=0.01) was observed in mice treated with both anti-CD19- and anti-CD79b CAR T cells (FIG. 6E). These results demonstrate that anti-CD79b CAR T cells have strong anti-tumor activity in vivo.

### Example 3—Further Development and Investigation of CD79b Antibodies and CARS

Binding of anti-CD79b antibodies to human CD79b: Full length anti-CD79b monoclonal antibodies (clones 5B and 28B) isolated from hybridoma supernatants were used to test binding to human CD79b expressed on mouse fibroblast L cells by ELISA (FIG. 7).

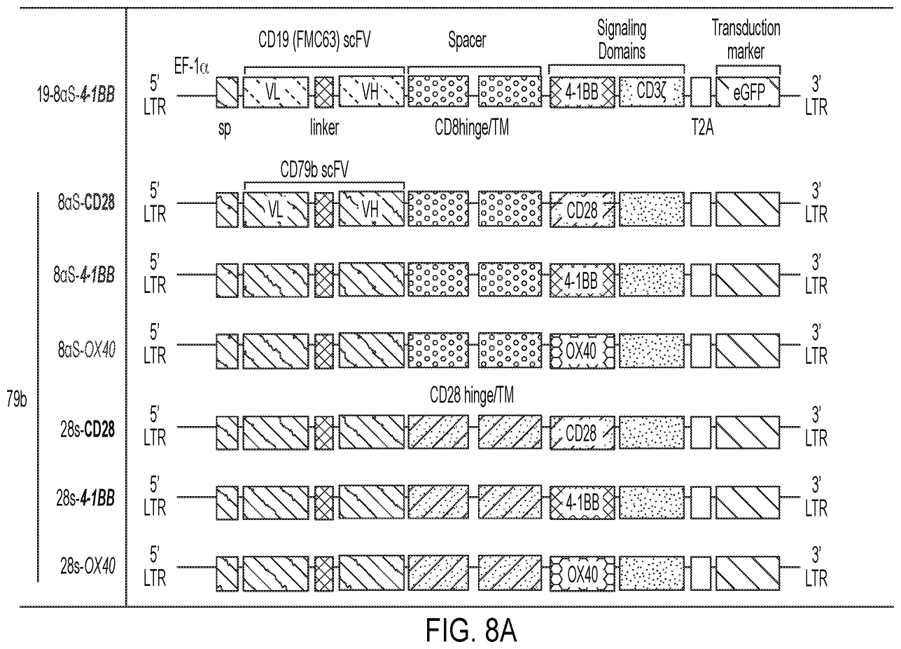

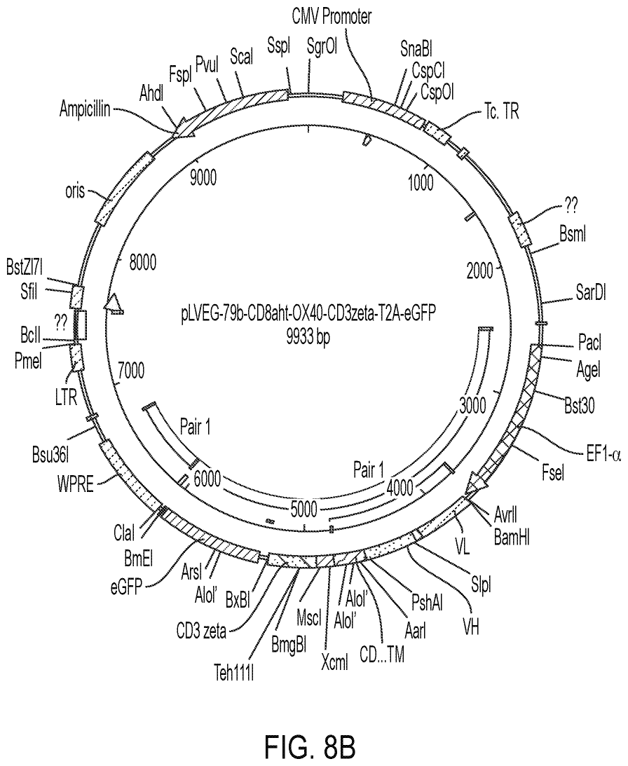

Generation of CAR constructs: Using the specific sequences of single chain fragments of variable region (scFv) derived from VH and VL of clone 28B (shown in FIG. 7), several constructs of anti-CD79b CAR were generated using either CD8α or CD28 hinge/transmembrane domains. In order to detect CAR expression in transduced T cells, CAR-enhanced green fluorescent protein (eGFP) fusion construct or the truncated human epidermal growth factor receptor (huEGFRt) were used (FIG. 8A). Anti-CD19 CAR construct was used as control. The latter can also serve as a safety switch to eliminate CAR T cells in case of severe toxicity. CD3-zeta (CD3) chain was incorporated to provide signal 1 for activation of T cells and the costimulatory domains CD28 or 4-1BB or OX40 were incorporated to provide signal 2 (FIG. 8A). These constructs were cloned into lentivirus vector pLVEG with EF1α promoter (FIG. 8B), which were then used to transduce T cells and tested in various in vitro and in vivo assays described below.

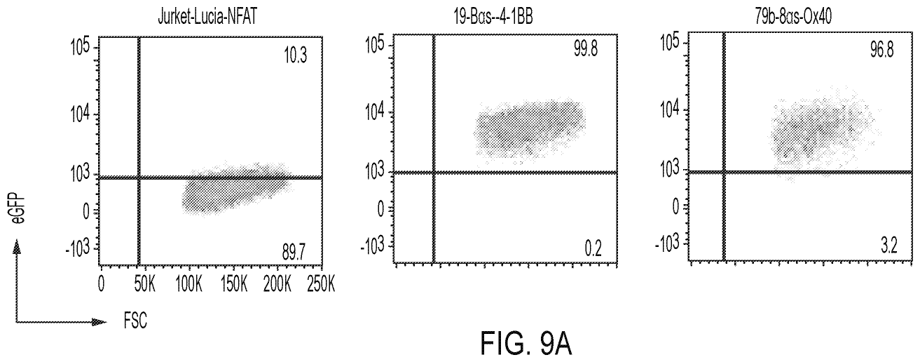

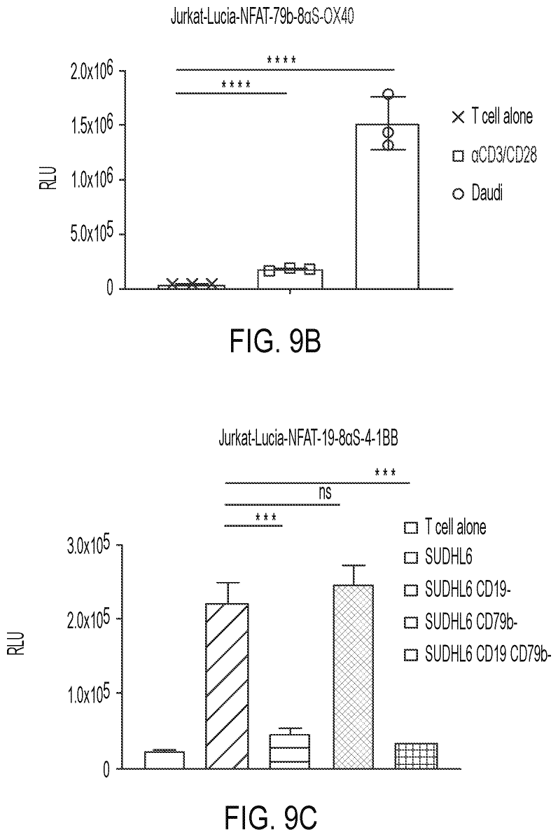

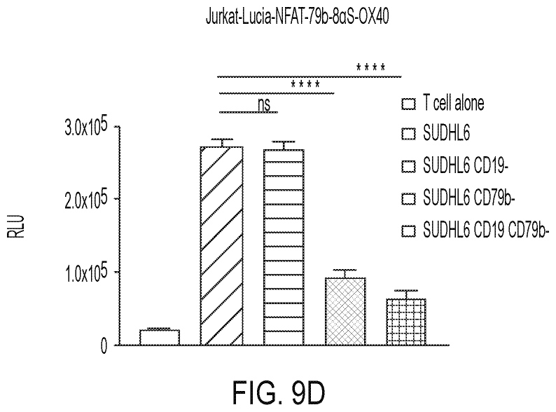

Anti-CD79b CARs specifically recognize human CD79b: To determine the signaling capability of anti-CD79b CARs, the CARs (clone 28B was used for CD79b CARs) were transduced using lentivirus into Jurkat-Lucia™ NFAT reporter cells (FIG. 9A). In this cell line, the Lucia gene, which encodes a secreted coelenterazine-utilizing luciferase, is driven by an ISG54 minimal promoter fused to six copies of the NFAT consensus transcriptional response element. Culture of these transduced Jurkat cells with anti-CD3/anti-CD28 antibodies or Daudi Burkitt lymphoma cells for 24 h induced strong luciferase activity compared to untreated cells (FIG. 9B). To establish the specificity of the recognition of CD79b by the CAR molecules, CRISPR/Cas9 was used to generate isogenic forms of SUDHL6, a diffuse large B-cell lymphoma cell line expressing CD19 and CD79b: parent or wild type (WT), CD19 knockout (CD19KO), CD79bKO, and CD19/CD79b double KO (CD19KO CD79bKO) (FIG. 9C). Co-culture of CAR-transduced Jurkat-Lucia™ NFAT reporter cells with SUDHL6 isogenic cell lines showed that CD79b CAR recognizes both parent and CD19KO cells but the reactivity was significantly diminished with CD79bKO or CD19KOCD79bKO cells. In contrast, the CD19 CAR recognized both parent and CD79bKO cells but not CD19KO or CD19KOCD79bKO cells (FIG. 9D).

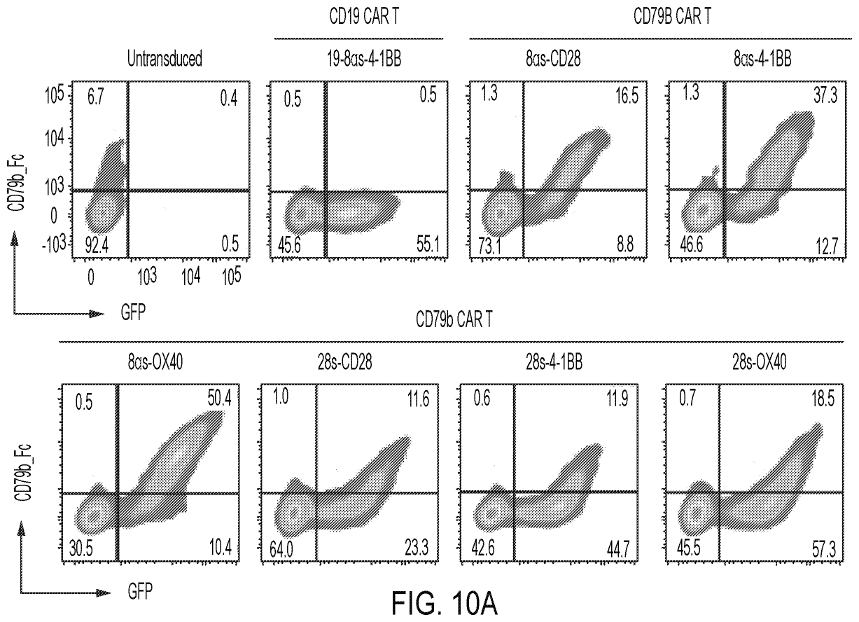

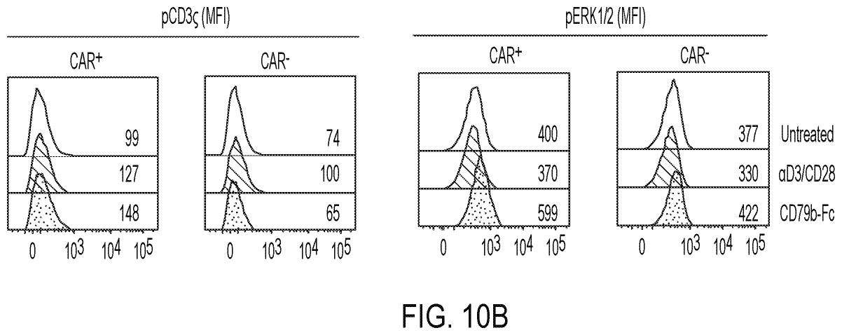

Transduction of CARs into primary human T cells: Representative transduction efficiency as determined by eGFP expression and/or recombinant human CD79b-Fc protein staining using the CAR constructs described above (FIG. 8A) is shown in FIG. 10A. Anti-CD19-CAR T cells were used as control. Anti-CD79b CAR T cells were either untreated or stimulated with anti-CD3/CD28 antibodies or recombinant human CD79b-Fc protein. After 15 mins, phosphorylation of CD3ζ and ERK1/2 was assessed by flow cytometry. The results show that there is no significant difference in baseline phosphorylation between CAR+ and CAR− T cells suggesting absence of tonic signaling. Stimulation with anti-CD3/CD28 antibodies induced phosphorylation of CD3ζ in both CAR+ but not CAR− T cells. In contrast, stimulation with recombinant human CD79b-Fc protein induced phosphorylation of CD3ζ and ERK1/2 in CAR+ but not CAR− T cells suggesting that the CARs can induced signaling in primary T cells in response to recognition of CD79b protein (FIG. 10B).

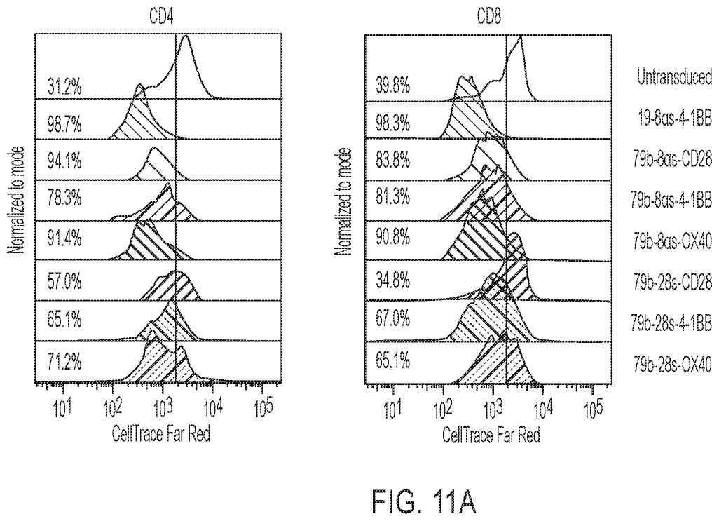

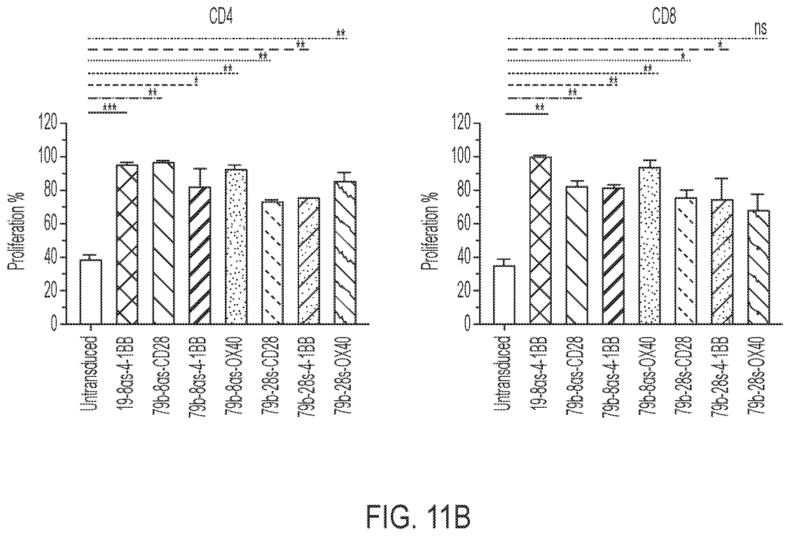

Proliferative activity of anti-CD79b CAR T cells: Anti-CD79b CAR T cells generated using above CAR constructs (FIG. 8A) from healthy donor T cells were labeled with CellTrace Far Red and co-cultured with CD79b-expressing Daudi Burkitt lymphoma tumor cells at an effector to target (E:T) ratio of 1:1. After 4 days, proliferation of CAR T cells was assessed by dye dilution method by flow cytometry. The results show that both CD4+ and CD8+ anti-CD79b CAR T cells proliferate significantly in response to Daudi tumor cells compared with untransduced T cells. The proliferation of anti-CD79b CAR T cells was comparable to anti-CD19 CART cells (FIGS. 11A and 11B).

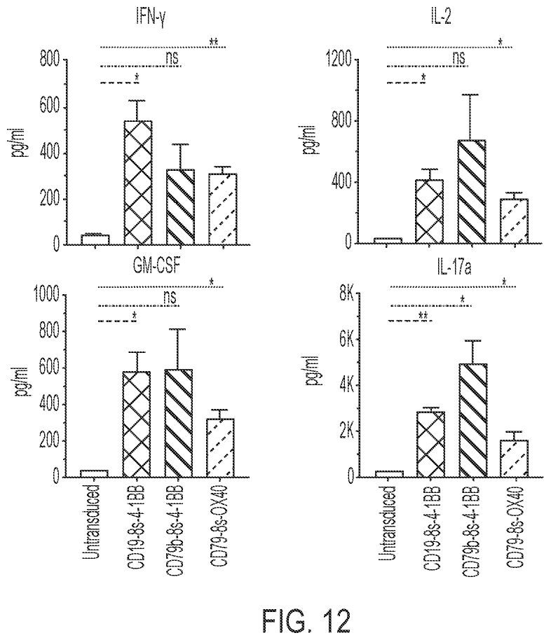

Cytokine production by anti-CD79b CAR T cells: Anti-CD79b CAR T cells generated using above CAR constructs (FIG. 8A) from healthy donor T cells were co-cultured with Daudi Burkitt lymphoma tumor cells at an E:T ratio of 1:1. After 24 hours, cytokine levels in the supernatants were assessed by multiplex cytokine assay. The results show that anti-CD79b CAR T cells produce significant amounts of IL-2, GM-CSF, IFN-γ, and IL-17A in response to Daudi tumor cells compared with untransduced T cells. The cytokine production by anti-CD79b CAR T cells was comparable to anti-CD19 CAR T cells (FIG. 12).

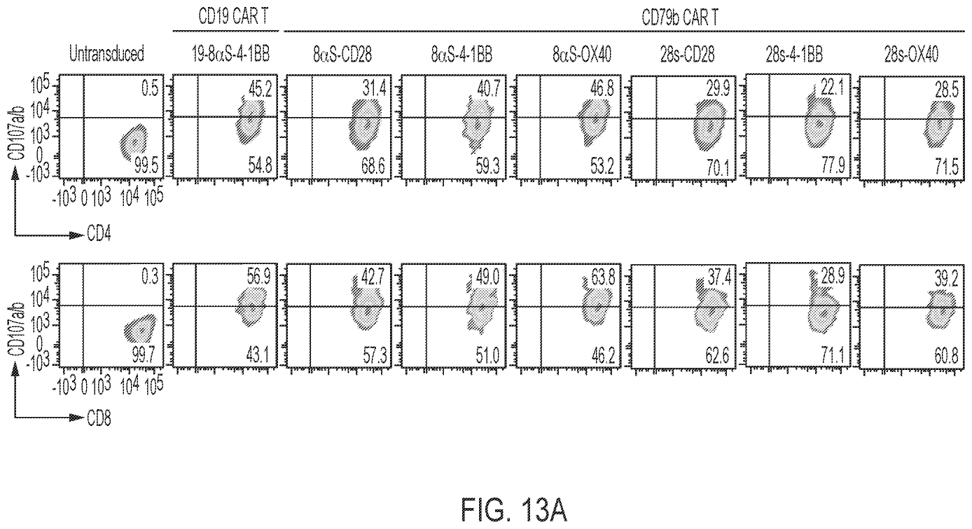

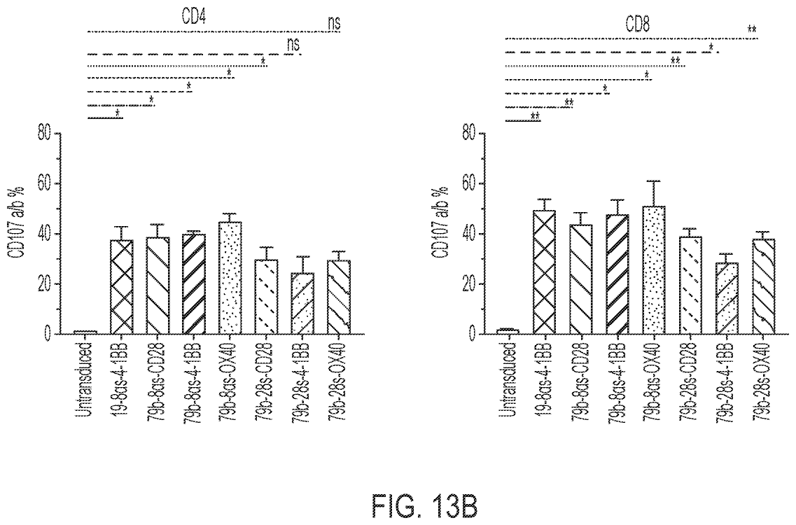

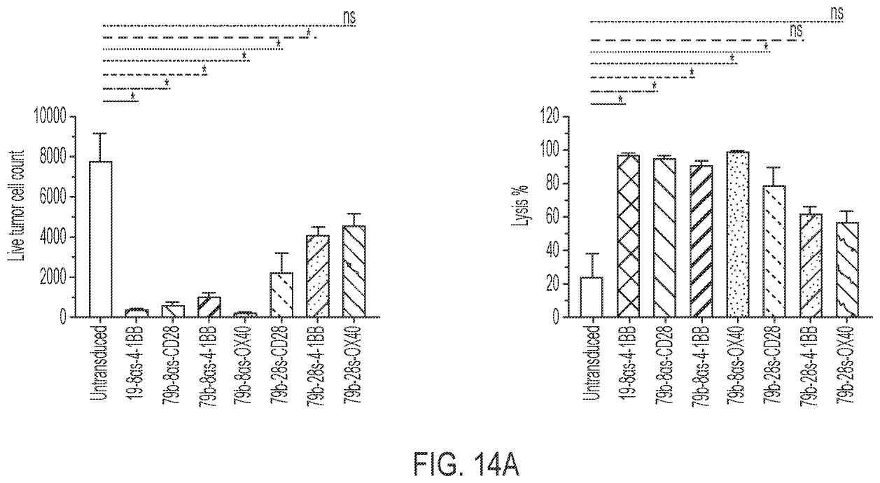

Anti-CD79b CAR T cells degranulate in response to lymphoma cells: Anti-CD79b CAR T cells generated using above CAR constructs (FIG. 8A) from healthy donor T cells were co-cultured with Daudi Burkitt lymphoma tumor cells at an E:T ratio of 1:1. After 6 hours, degranulation was assessed by flow cytometry after staining for CD107 a/b. The results show that both CD4+ and CD8+ anti-CD79b CAR T cells degranulate significantly in response to Daudi tumor cells compared with untransduced T cells. The degranulation by anti-CD79b CAR T cells was comparable to anti-CD19 CART cells. The degranulation was numerically superior with anti-CD79b CAR T cells containing CD8α hinge/transmembrane domain compared with anti-CD79b CAR T cells containing CD28 hinge/transmembrane domain (FIGS. 13A and 13B).

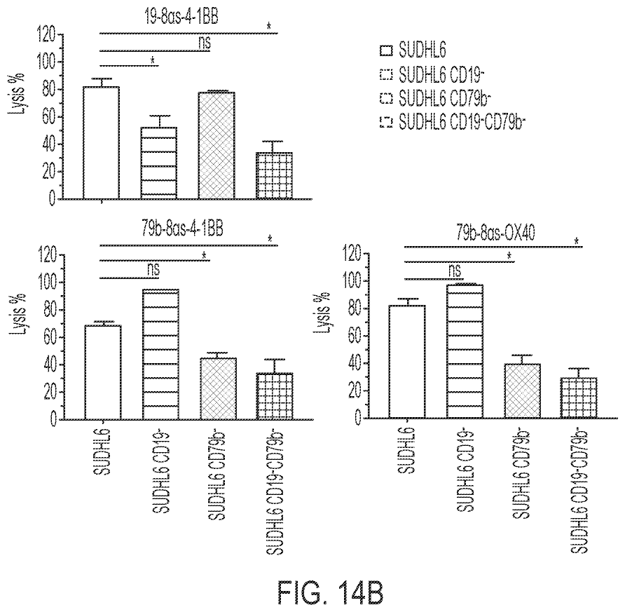

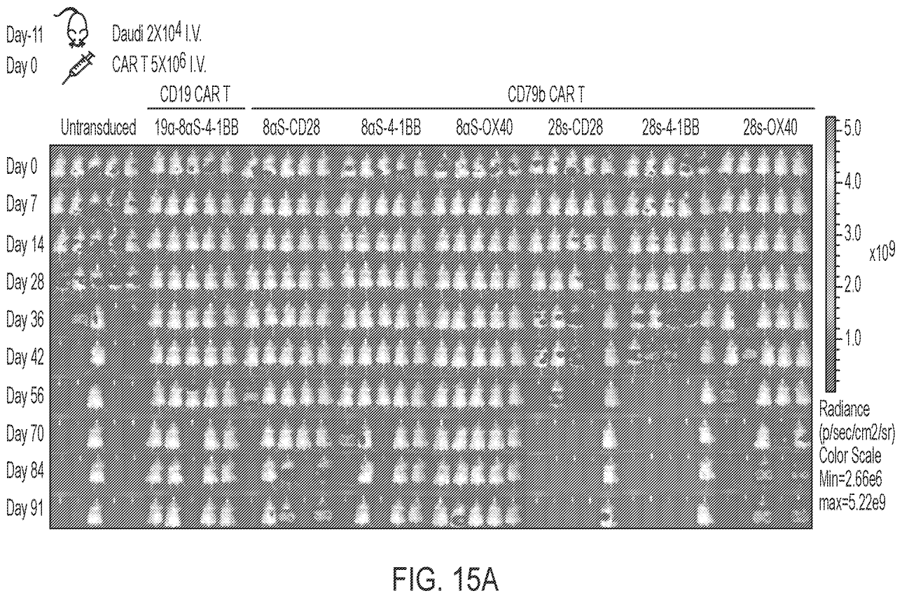

Anti-CD79b CAR T cells are cytotoxic to lymphoma cells: Anti-CD79b CAR T cells generated using above CAR constructs (FIG. 8A) from healthy donor T cells and labeled with CellTrace Far Red were co-cultured with Daudi Burkitt lymphoma tumor cells or SUDHL6 isogenic cell lines (parent, CD19KO, CD79bKO, and CD19/CD79b double KO labeled with CellTrace Violet at an E:T ratio of 1:1. After 4 days, cytotoxicity was assessed by flow cytometry by determining the absolute number of residual live tumor cells and calculating percent lysis. The results show that anti-CD79b CAR T cells induce significant cytotoxicity of Daudi tumor cells compared with untransduced T cells. The cytotoxic activity of anti-CD79b CAR T cells was comparable to anti-CD19 CAR T cells. The cytotoxic activity was superior with anti-CD79b CAR T cells containing CD8α hinge/transmembrane domain compared with anti-CD79b CAR T cells containing CD28 hinge/transmembrane domain (FIG. 14A). Furthermore, anti-CD79b CART cells induced significant lysis of both parent and CD19KO SUDHL6 cells but the lysis was significantly diminished with CD79bKO or CD19KOCD79bKO cells. In contrast, the anti-CD19 CAR T cells induced significant lysis of both parent and CD79bKO cells but lysis was significantly diminished with CD19KO or CD19KOCD79bKO cells (FIG. 14B).

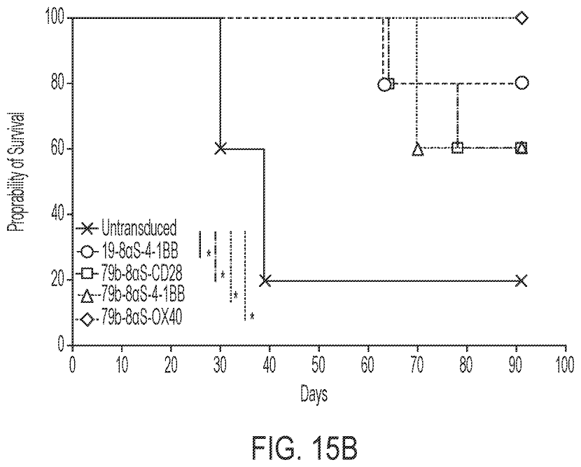

Anti-CD79b CAR T cells exert antitumor effects in vivo: To examine the efficacy of anti-CD79b-CAR T cells in vivo, Daudi Burkitt lymphoma cells expressing firefly luciferase gene were injected IV into NSG mice at 2×10⁴ tumor cells/mouse. After 11 days, mice were treated with untransduced or anti-CD19-CAR T cells or anti-CD79b-CAR T cells generated using above CAR constructs (FIG. 8A) via tail vein injection at 5×10⁶ CAR+ T cells/mouse. Bioluminescence imaging was used to assess tumor burden (FIG. 15A). The results showed rapid tumor growth in mice treated with untransduced T cells. In contrast, good tumor control and markedly improved survival were seen in mice treated with both anti-CD19 CAR and anti-CD79b-CAR T cells. The tumor control and survival was superior with anti-CD79b CAR T cells containing CD8α hinge/transmembrane domain compared with anti-CD79b CAR T cells containing CD28 hinge/transmembrane domain with the best survival observed with anti-CD79b CAR T cells containing CD8α hinge/transmembrane domain and OX40 costimulatory domain (FIG. 15B).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Ausubel et al., Current Protocols in Molecular Biology, Greene Publishing Associates and John Wiley & Sons, N Y, 1994.
Chothia et al., 1988.
Davila et al., 2013.
European patent application number EP2537416
Heemskerk et al. Hum Gene Ther. 19:496-510, 2008.
International Patent Publication No. WO/2014055668 A1.
International Patent Publication No. WO200014257
International Patent Publication No. WO2012/129514
International Patent Publication No. WO2013/071154
International Patent Publication No. WO2013/123061
International Patent Publication No. WO2013/166321
International Patent Publication No. WO2013126726
International Patent Publication No. WO2014031687
Johnson et al. Blood 114:535-46, 2009.
Jores et al., 1990.
Kabat et al., “Sequences of Proteins of Immunological Interest, US Dept. Health and Human Services, Public Health Service National Institutes of Health, 1991, 5^{th} ed.
Lefranc et al., 2003.
Liu et al., 2003.
Remington's Pharmaceutical Sciences 22^{nd} edition, 2012.
Sadelain et al., 2013.
Sambrook et al., Molecular Cloning: A Laboratory Manual, 3^{rd} ed., Cold Spring Harbor Press, Cold Spring Harbor, N.Y. 2001.
Turtle et al., 2012.
U.S. Pat. No. 5,091,513
U.S. Pat. No. 5,091,513
U.S. Pat. No. 5,994,136
U.S. Pat. No. 6,013,516
U.S. Pat. No. 6,410,319
U.S. Pat. No. 6,410,319
U.S. Pat. No. 6,451,995
U.S. Pat. No. 6,881,557
U.S. Pat. No. 6,946,546
U.S. Pat. No. 7,070,995
U.S. Pat. No. 7,265,209
U.S. Pat. No. 7,354,762
U.S. Pat. No. 7,446,179
U.S. Pat. No. 7,446,190
U.S. Pat. No. 7,446,191
U.S. Pat. No. 8,252,592
U.S. Pat. No. 8,324,353
U.S. Pat. No. 8,339,645
U.S. Pat. No. 8,398,282
U.S. Pat. No. 8,479,118
U.S. Pat. No. 7,446,190
U.S. Patent Publication No. US2002131960
U.S. Patent Publication No. US20050214860
U.S. Patent Publication No. US20130149337
U.S. Patent Publication No. US2013287748
Wu et al., 2012

What is claimed is:

1. An isolated monoclonal antibody, wherein the antibody specifically binds to CD79b and comprises:
(I):
(a) a first V_{H} CDR comprising SEQ ID NO: 1;
(b) a second V_{H} CDR comprising SEQ ID NO: 2;
(c) a third V_{H} CDR comprising SEQ ID NO: 3;
(d) a first V_{L} CDR comprising SEQ ID NO: 4;
(e) a second V_{L} CDR comprising SEQ ID NO: 5; and
(f) a third V_{L} CDR comprising SEQ ID NO: 6;
(II):
(a) a first V_{H} CDR comprising SEQ ID NO: 11;
(b) a second V_{H} CDR comprising SEQ ID NO: 12;
(c) a third V_{H} CDR comprising SEQ ID NO: 13;
(d) a first V_{L} CDR comprising SEQ ID NO: 14;
(e) a second V_{L} CDR comprising SEQ ID NO: 15; and
(f) a third V_{L} CDR comprising SEQ ID NO: 16; or
(III):
(a) a first V_{H} CDR comprising SEQ ID NO: 21;
(b) a second V_{H} CDR comprising SEQ ID NO: 22;
(c) a third V_{H} CDR comprising SEQ ID NO: 23;
(d) a first V_{L} CDR comprising SEQ ID NO: 24;
(e) a second V_{L} CDR comprising SEQ ID NO: 25; and
(f) a third V_{L} CDR comprising SEQ ID NO: 26.

2. The antibody of claim 1, wherein the antibody comprises:
(a) a first V_{H} CDR comprising SEQ ID NO: 1;
(b) a second V_{H} CDR comprising SEQ ID NO: 2;
(c) a third V_{H} CDR comprising SEQ ID NO: 3;
(d) a first V_{L} CDR comprising SEQ ID NO: 4;
(e) a second V_{L} CDR comprising SEQ ID NO: 5; and
(f) a third V_{L} CDR comprising SEQ ID NO: 6;

3. The antibody of claim 2, wherein the antibody comprises a V_{H} domain at least about 80% identical to the V_{H} domain of SEQ ID NO: 7 and a V_{L} domain at least about 80% identical to the V_{L} domain of SEQ ID NO: 9.

4. The antibody of claim 2, wherein the antibody comprises a V_{H} domain identical to the V_{H} domain of SEQ ID NO: 7 and a V_{L} domain identical to the V_{L} domain of SEQ ID NO: 9.

5. The antibody of claim 1, wherein the antibody comprises:
(a) a first V_{H} CDR comprising SEQ ID NO: 11;
(b) a second V_{H} CDR comprising SEQ ID NO: 12;
(c) a third V_{H} CDR comprising SEQ ID NO: 13;
(d) a first V_{L} CDR comprising SEQ ID NO: 14;
(e) a second V_{L} CDR comprising SEQ ID NO: 15; and
(f) a third V_{L} CDR comprising SEQ ID NO: 16.

6. The antibody of claim 5, wherein the antibody comprises a V_{H} domain at least about 80% identical to the V_{H} domain of SEQ ID NO: 17 and a V_{L} domain at least about 80% identical to the V_{L} domain of SEQ ID NO: 19.

7. The antibody of claim 5, wherein the antibody comprises a V_{H} domain identical to the V_{H} domain of SEQ ID NO: 17 and a V_{L} domain identical to the V_{L} domain SEQ ID NO: 19.

8. The antibody of claim 1, wherein the antibody comprises:
(a) a first V_{H} CDR comprising a sequence that is SEQ ID NO: 21;
(b) a second V_{H} CDR comprising SEQ ID NO: 22;
(c) a third V_{H} CDR comprising SEQ ID NO: 23;
(d) a first V_{L} CDR comprising SEQ ID NO: 24;
(e) a second V_{L} CDR comprising SEQ ID NO: 25; and
(f) a third V_{L} CDR comprising SEQ ID NO: 26.

9. The antibody of claim 8, wherein the antibody comprises a V_{H} domain at least about 80% identical to the V_{H} domain of SEQ ID NO: 27 and a V_{L} domain at least about 80% identical to the V_{L} domain of SEQ ID NO: 29.

10. The antibody of claim 8, wherein the antibody comprises a V_{H} domain identical to the V_{H} domain of SEQ ID NO: 27 and a V_{L} domain identical to the V_{L} domain SEQ ID NO: 29.

11. The antibody of any one of claims 1-10, wherein the antibody is recombinant.

12. The antibody of claim 1, wherein the antibody is an IgG, IgM, IgA or an antigen binding fragment thereof.

13. The antibody of any one of claims 1-10, wherein the antibody is a Fab′, a F(ab′)2, a F(ab′)3, a monovalent scFv, a bivalent scFv, or a single domain antibody.

14. The antibody of any one of claims 1-12, wherein the antibody is a human, humanized antibody or de-immunized antibody.

15. The antibody of any one of claims 1-14, wherein the antibody is conjugated to an imaging agent, a chemotherapeutic agent, a toxin or a radionuclide.

16. A composition comprising an antibody of any one of claims 1-15 in a pharmaceutically acceptable carrier.

17. An isolated polynucleotide molecule comprising a nucleic acid sequence encoding an antibody of any one of claims 1-14.

18. A recombinant polypeptide comprising an antibody V_{H} domain comprising CDRs 1-3 of the V_{H} domain of Clone T26 (SEQ ID NOs: 1, 2, and 3) and CDRs 1-3 of the V_{L} domain of Clone T26 (SEQ ID NOs: 4, 5, and 6).

19. A recombinant polypeptide comprising an antibody V_{H} domain comprising CDRs 1-3 of the V_{H} domain of Clone 5B (SEQ ID NOs: 11, 12, and 13) and CDRs 1-3 of the V_{L} domain of Clone 5B (SEQ ID NOs: 14, 15, and 16).

20. A recombinant polypeptide comprising an antibody V_{H} domain comprising CDRs 1-3 of the V_{H} domain of Clone 28B (SEQ ID NOs: 21, 22, and 23) and CDRs 1-3 of the V_{L} domain of Clone 28B (SEQ ID NOs: 24, 25, and 26).

21. An isolated polynucleotide molecule comprising a nucleic acid sequence encoding a polypeptide of any of claims 18-20.

22. A host cell comprising one or more polynucleotide molecule(s) encoding an antibody of any one of claims 1-14 or a recombinant polypeptide of any of claims 18-20.

23. The host cell of claim 22, wherein the host cell is a mammalian cell, a yeast cell, a bacterial cell, a ciliate cell or an insect cell.

24. A method for treating a subject having a cancer comprising administering an effective amount of an antibody of any one of claims 1-13 to the subject.

25. The method of claim 24, wherein the cancer is B cell malignancy.

26. The method of claim 24, wherein the antibody is in a pharmaceutically acceptable composition.

27. The method of claim 24, wherein the antibody is administered systemically.

28. The method of claim 24, wherein the antibody is administered intravenously, intradermally, intratumorally, intramuscularly, intraperitoneally, subcutaneously, or locally.

29. The method of claim 24, further comprising administering at least a second anticancer therapy to the subject.

30. The method of claim 29, wherein the second anticancer therapy is a surgical therapy, chemotherapy, radiation therapy, cryotherapy, hormonal therapy, immunotherapy or cytokine therapy.

31. The method of claim 29, wherein the second anticancer therapy comprises an adoptive T-cell therapy.

32. An engineered CD79b CAR or TCR having an antigen binding domain comprising:
(I):
(a) a first V_{H} CDR comprising SEQ ID NO: 1;
(b) a second V_{H} CDR comprising SEQ ID NO: 2;
(c) a third V_{H} CDR comprising SEQ ID NO: 3;
(d) a first V_{L} CDR comprising SEQ ID NO: 4;
(e) a second V_{L} CDR comprising SEQ ID NO: 5; and
(f) a third V_{L} CDR comprising SEQ ID NO: 6;
(II):
(a) a first V_{H} CDR comprising SEQ ID NO: 11;
(b) a second V_{H} CDR comprising SEQ ID NO: 12;
(c) a third V_{H} CDR comprising SEQ ID NO: 13;
(d) a first V_{L} CDR comprising SEQ ID NO: 14;
(e) a second V_{L} CDR comprising SEQ ID NO: 15; and
(f) a third V_{L} CDR comprising SEQ ID NO: 16; or
(III):
(a) a first V_{H} CDR comprising SEQ ID NO: 21;
(b) a second V_{H} CDR comprising SEQ ID NO: 22;
(c) a third V_{H} CDR comprising SEQ ID NO: 23;
(d) a first V_{L} CDR comprising SEQ ID NO: 24;
(e) a second V_{L} CDR comprising SEQ ID NO: 25; and
(f) a third V_{L} CDR comprising SEQ ID NO: 26.

33. The CAR or TCR of claim 32, wherein the antigen-binding domain comprises:
(a) a first V_{H} CDR comprising SEQ ID NO: 1;
(b) a second V_{H} CDR comprising SEQ ID NO: 2;
(c) a third V_{H} CDR comprising SEQ ID NO: 3;
(d) a first V_{L} CDR comprising SEQ ID NO: 4;
(e) a second V_{L} CDR comprising SEQ ID NO: 5; and
(f) a third V_{L} CDR comprising SEQ ID NO: 6.

34. The CAR or TCR of claim 33, wherein the antigen-binding domain comprises a V_{H} domain at least about 80% identical to the V_{H} domain of SEQ ID NO: 7 and a V_{L} domain at least about 80% identical to the V_{L} domain of SEQ ID NO: 9.

35. The CAR or TCR of claim 33, wherein the antigen-binding domain comprises a V_{H} domain identical to the V_{H} domain of SEQ ID NO: 7 and a V_{L} domain identical to the V_{L} domain of SEQ ID NO: 9.

36. The CAR or TCR of claim 32, wherein the antibody comprises:
(a) a first V_{H} CDR comprising SEQ ID NO: 11;
(b) a second V_{H} CDR comprising SEQ ID NO: 12;
(c) a third V_{H} CDR comprising SEQ ID NO: 13;
(d) a first V_{L} CDR comprising SEQ ID NO: 14;
(e) a second V_{L} CDR comprising SEQ ID NO: 15; and
(f) a third V_{L} CDR comprising SEQ ID NO: 16.

37. The CAR or TCR of claim 36, wherein the antigen-binding domain comprises a V_{H} domain at least about 80% identical to the V_{H} domain of SEQ ID NO: 17 and a V_{L} domain at least about 80% identical to the V_{L} domain of SEQ ID NO: 19.

38. The CAR or TCR of claim 36, wherein the antigen-binding domain comprises a V_{H} domain identical to the V_{H} domain of SEQ ID NO: 17 and a V_{L} domain identical to the V_{L} domain SEQ ID NO: 19.

39. The CAR or TCR of claim 32, wherein the antigen-binding domain comprises:
(a) a first V_{H} CDR comprising SEQ ID NO: 21;
(b) a second V_{H} CDR comprising SEQ ID NO: 22;
(c) a third V_{H} CDR comprising SEQ ID NO: 23;
(d) a first V_{L} CDR comprising SEQ ID NO: 24;
(e) a second V_{L} CDR comprising SEQ ID NO: 25; and
(f) a third V_{L} CDR comprising SEQ ID NO: 26.

40. The CAR or TCR of claim 39, wherein the antigen-binding domain comprises a V_{H} domain at least about 80% identical to the V_{H} domain of SEQ ID NO: 27 and a V_{L} domain at least about 80% identical to the V_{L} domain of SEQ ID NO: 29.

41. The CAR or TCR of claim 39, wherein the antigen-binding domain comprises a V_{H} domain identical to the V_{H} domain of SEQ ID NO: 27 and a V_{L} domain identical to the V_{L} domain SEQ ID NO: 29.

42. The CAR or TCR of claim 32, wherein the CAR comprises one or more signaling domains selected from the group consisting of CD3ξ, CD28, OX40/CD134, 4-1BB/CD137, and a combination thereof.

43. The CAR or TCR of claim 32, wherein the CAR comprises CD3ζ and CD28 signaling domains.

44. The CAR or TCR of claim 32, wherein the CAR comprises CD3ζ and 4-1BB signaling domains.

45. The CAR or TCR of claim 32, wherein the CAR comprises CD3ζ and OX-40 signaling domains.

46. The CAR or TCR of claim 32, wherein the CAR or TCR is encoded by a viral vector.

47. The CAR or TCR of claim 46, wherein the viral vector is a lentiviral vector.

48. The CAR or TCR of claim any of claims 32-80, wherein the antigen-binding domain comprises a V_{H} domain linked to a V_{L} domain by a linker.

49. The CAR or TCR of claim 48, wherein the linker comprises Linker 1 (SEQ ID NO: 45) or a linker encoded by the polynucleotide of SEQ ID NO: 44, Linker 2 (SEQ ID NO: 47) or a linker encoded by the polynucleotide of SEQ ID NO: 46, Linker 3 (SEQ ID NO: 49) or a linker encoded by the polynucleotide of SEQ ID NO: 48, or Linker 4 (SEQ ID NOs: 51) or a linker encoded by the polynucleotide of SEQ ID NO: 50.

50. The CAR or TCR of any of claims 32-49, wherein the CAR comprises V_{L}-Linker1-V_{H}, V_{L}-Linker2-V_{H}, V_{L}-Linker3-V_{H}, V_{L}-Linker4-V_{H}, V_{H}-Linker1-V_{L}, V_{H}-Linker2-V_{L}, V_{H}-Linker3-V_{L}, or V_{H}-Linker4-V_{L}.

51. The CAR or TCR of any of claims 32-50, wherein the CAR or TCR comprises a hinge.

52. The CAR or TCR of claim 51, wherein the hinge is CD8 Hinge 1 (SEQ ID NO: 53), or a hinge encoded by the polynucleotide of SEQ ID NO: 52, CD8 Hinge 2 (SEQ ID NO: 55) or a hinge encoded by the polynucleotide of SEQ ID NO: 54, CD8 Hinge 3 (SEQ ID NO: 57) or a hinge encoded by the polynucleotide of SEQ ID NO: 56, CD28 Hinge (SEQ ID NO: 59) or a hinge encoded by the polynucleotide of SEQ ID NO: 58, IgG4 Hinge (SEQ ID NOs:60 or 61), IgG4 CH2 (SEQ ID NO: 63) or a hinge encoded by the polynucleotide of SEQ ID NO: 62, IgG4 CH2CH3 (SEQ ID NO: 65) or a hinge encoded by the polynucleotide of SEQ ID NO: 64, or IgG4 CH1CH2CH3 (SEQ ID NO: 67) or a hinge encoded by the polynucleotide of SEQ ID NO: 66.

53. The CAR or TCR of any of claims 32-52, wherein the CAR comprises a transmembrane domain.

54. The CAR or TCR of claim 53, wherein the transmembrane domain is CD8 TM1 (SEQ ID NO: 69) or a transmembrane domain encoded by the polynucleotide of SEQ ID NO: 68, CD8 TM2 (SEQ ID NO: 71) or a transmembrane domain encoded by the polynucleotide of SEQ ID NO: 70, CD28 TM (SEQ ID NO: 73) or a transmembrane domain encoded by the polynucleotide of SEQ ID NO: 72, or CD8α TM (SEQ ID No: 87) or a transmembrane domain encoded by the polynucleotide of SEQ ID NO: 86.

55. The CAR or TCR of claim 32, further comprising a transduction marker and/or safety switch.

56. The CAR or TCR of claim 55, wherein the transduction marker is enhanced green fluorescent protein (eGFP).

57. The CAR or TCR of claim 56, wherein the eGFP has an amino acid sequence of SEQ ID NO:83.

58. The CAR or TCR of claim 55, wherein the transduction marker and/or safety switch is truncated epidermal growth factor (EGFR).

59. The CAR or TCR of claim 58, wherein the EGFR has an amino acid sequence of SEQ ID NO:41.

60. The CAR or TCR of claim 55, wherein the transduction marker and/or safety switch is linked to the CAR by a cleavage peptide.

61. The CAR or TCR of claim 60, wherein the cleavage peptide is a 2A peptide.

62. The CAR or TCR of claim 61, wherein the 2A peptide if a T2A peptide.

63. The CAR or TCR of claim 62, wherein the T2A peptide has an amino acid sequence of SEQ ID NO:85.

64. The CAR or TCR of claim 32, wherein the CAR further comprises a second antigen binding domain.

65. The CAR or TCR of claim 64, wherein the second antigen binding domain is a CD19, CD20, or CD22 antigen binding domain.

66. The CAR or TCR of any one of claims 32-65, wherein the CAR or TCR comprises a CD8α hinge and transmembrane domain comprising SEQ ID NO: 87 or a transmembrane domain encoded by the polypeptide of SEQ ID NO: 86, a human OX-40 signalling domain comprising SEQ ID NO: 79 or a signaling domain encoded by the polypeptide of SEQ ID NO: 78, and a CD3ζ domain comprising SEQ ID NO: 81 or a domain encoded by the polypeptide of SEQ ID NO: 81.

67. An expression vector encoding the CAR or TCR of any one of claims 32-66.

68. A host cell engineered to express a CD79b CAR or a CD79b TCR.

69. The cell of claim 68, wherein the cell is engineered to express a CAR of any one of claims 30-65.

70. The cell of claim 68, wherein the host cell is an immune cell.

71. The cell of claim 70, wherein the immune cell is a T cell.

72. The cell of claim 71, wherein the T cell is a primary human T cell or a TIL.

73. The cell of claim 71, wherein the T cell is a CD4+ T cell or CD8+ T cell.

74. The cell of claim 72, wherein the primary human T cell is obtained from a healthy donor.

75. The cell of claim 71, wherein the T cell is autologous.

76. The cell of claim 71, wherein the T cell is allogeneic.

77. The cell of claim 68, wherein the cell is engineered using a CRISPR or transposase system.

78. A pharmaceutical composition comprising CD79b targeted T cells and a pharmaceutical carrier, wherein the CD79b targeted T cells are engineered to express a CAR or TCR of any one of claims 32-66.

79. A composition comprising an effective amount of CD79b targeted T cells for the treatment of cancer in a subject, wherein the CD79b targeted T cells are engineered to express a CAR or TCR of any one of claims 32-66.

80. A use of a composition comprising an effective amount of CD79b targeted T cells for the treatment of cancer in a subject, wherein the CD79b targeted T cells are engineered to express a CAR or TCR of any one of claims 32-66.

81. A method for treating cancer in a subject comprising administering an effective amount of CD79b targeted T cells to the subject, wherein the CD79b targeted T cells are engineered to express a CAR or TCR of any one of claims 32-66.

82. The method of claim 81, wherein the cancer is a B cell malignancy.

83. The method of claim 82, wherein the B cell malignancy is B cell acute lymphoblastic leukemia (ALL), diffuse, large B cell lymphoma, follicular lymphoma, marginal zone lymphoma, lymphoplasmacytic lymphoma, Burkitt lymphoma, or chronic lymphocytic leukemia.

84. The method of claim 81, wherein the subject has been previously administered a CD19 CAR therapy.

85. The method of claim 84, wherein the subject is resistant to CD19 CAR therapy.

86. The method of claim 85, wherein the subject has CD19 antigen loss.

87. The method of claim 86, wherein the subject has relapsed with a CD19-negative tumor.

88. The method of claim 81, wherein the CD79b targeted T cells are administered intravenously, intradermally, intratumorally, intramuscularly, intraperitoneally, subcutaneously, or locally.

89. The method of claim 81, wherein the CD79b targeted T cells are administered intravenously.

90. The method of claim 81, further comprising administering at least a second anticancer therapy to the subject.

91. The method of claim 90, wherein the second anticancer therapy is a surgical therapy, chemotherapy, radiation therapy, cryotherapy, hormonal therapy, immunotherapy or cytokine therapy.

92. The method of claim 81, wherein the cancer is a CD79b-expressing cancer.
